(19) United States
(12) Patent Application Publication
(10) Pub. No.: US 2011/0165265 A1
(43) Pub. Date: Jul. 7, 2011

(54) TRAIL VARIANTS FOR TREATING CANCER

(75) Inventor: Afshin Samali, Galway (IE)

(73) Assignees: FUNDACIÓ GENÒMICA (CRG), Barcelona (ES); INSTITUCIÓ CATALANA DE RECERCA I ESTUDIS AVANCATS, Barcelona (ES); NATIONAL UNIVERSITY OF IRELAND, GALWAY, Galway (IE); ACADEMISCH ZIEKENHUIS GRONINGEN, Groningen (NL); RIJKSUNIVERSITEIT GRONINGEN, Groningen (NL)

(21) Appl. No.: 12/808,595

(22) PCT Filed: Dec. 17, 2008
(86) PCT No.: PCT/IB2008/003720

§ 371 Date: Mar. 9, 2011

(30) Foreign Application Priority Data
Dec. 17, 2007 (GB) 0724532.7

(51) Int. Cl. A61K 33/24; A61K 38/17; A61P 35/00

(52) U.S. Cl. 424/649; 514/19.3

(57) ABSTRACT

The present invention relates to the use of a mutant TRAIL protein to treat various cancers in mammals. The invention provides a variant TRAIL protein, which has superior selectivity for the death receptor 5, for treating a mammal diagnosed with cancer.

The present invention relates to the use of a mutant TRAIL protein to treat various cancers in mammals.

Cytokines are a family of growth factors, secreted primarily from leukocytes, and are messenger proteins that act as potent regulators, capable of controlling a wide range of cellular functions, especially the immune response and cell growth¹. These roles include immune response regulation², inflammation³, wound healing⁴, embryogenesis and development, and apoptosis⁵.

Of particular interest are ligands that belong to the Tumor Necrosis Factor ligand (TNF) family; these proteins are involved in a wide range of biological activities, ranging from cell proliferation to apoptosis. One example of a member of the TNF family is TRAIL (tumor necrosis factor-related apoptosis inducing ligand).

TRAIL can interact with several different receptors on the cell. Two of those, DR4 (TRAIL-R1) and DR5 (TRAIL-R2) can induce apoptosis in the cell, while the others DcR1 (TRAIL-R3) and DcR2 (TRAIL-R4), do not contain a death domain or contain a truncated death domain, respectively, and are not able to induce apoptosis.

TRAIL, in its soluble form, selectively induces apoptosis in tumor cells in vitro and in vivo. Unlike other apoptosis inducing TNF family members, TRAIL appears to be inactive against normal healthy tissue, therefore attracting great interest as a potential cancer therapeutic⁶. Therefore TRAIL has the potential to serve as a safe and potent therapeutic agent against tumor cells. A number of in vitro studies have shown that many tumor cell lines of divergent origins are sensitive to TRAIL induced apoptosis.

A recent significant publication has shown that DcR1 (TRAIL-R3) is upregulated by p53 in breast tumor cells through use'of the genotoxic drug, doxorubicin⁷. This implies that efficacy of wild-type TRAIL (wtTRAIL) may be diminished in anti-tumor therapy since it also binds the decoy receptors (that do not initiate apoptosis). Therefore, variants of TRAIL that have altered selectivity/specificity might, in theory, have ultimately improved application in cancer treatment.

In this respect, selectivity of novel molecules is of primary importance to discern the specific role of the activation of different receptors and therefore the functional effects of ligand binding to several receptors, and the concomitant influence on the pathogenesis of the associated diseases related to signal activation.

### SUMMARY OF THE INVENTION

According to a first aspect of the invention, there is provided a variant TRAIL protein, which has superior selectivity for the death receptor 5, for treating a mammal diagnosed with cancer.

TRAIL (also known as TNFSF10, TL2; APO2L; CD253; Apo-2L) is a member of the TNF ligand family^{8 9} and an example of a cytokine that binds to more than one receptor, including decoy receptors, which lack or have truncated intracellular domains. TRAIL is a promiscuous ligand as it binds to five cognate receptors of the TNF receptor family; to the death receptor 4 (DR4, TRAIL-R1), death receptor 5 (DR5, TRAIL-R2, KILLLER, TRICK-2) and to the decoy receptor 1, (DcR1, TRAIL-R3, TRIDD), decoy receptor 2 (DcR2, TRAIL-R4, TRUNDD) and to the soluble secreted receptor osteoprotegerin (OPG). Only DR4 (TRAIL-RI) and DR5 (TRAIL-R2), and not DcR1, DcR2 or OPG contain a functional death domain (DD) and thus, only binding of TRAIL to these receptors induces apoptosis via activation of a cell—extrinsic or death receptor-mediated apoptosis pathway. Through the same receptors as well as through DcR2, TRAIL also appears to be able to induce the pro-survival NF-κB pathway. The factors that determine which pathway (apoptosis or survival) dominates in a given cell are poorly understood.

The provision of selective inducers of DR5 (TRAIL-R2) signalling is potentially of great interest, given the different cross-linking requirements of both death receptors DR4 and DR5. For example, depending on the cross-linking, the signalling pathway could induce the proliferative or the apoptotic pathway. Furthermore, it has been reported that binding of TRAIL to the decoy receptors can have an inhibitory effect on the efficiency of TRAIL to induce apoptosis. The inventors are therefore of the view that the use of DR5-receptor selective TRAIL variants could permit better tumor-specific therapies through escape from the decoy receptor-mediated antagonism, resulting in a higher efficacy with possibly less side effects as compared to wtTRAIL.

Furthermore, it seems that receptors DR5 and/or DR4 may be up-regulated after treatment with DNA damaging chemotherapeutic drugs such as Bortezomib, 5-Flurouracil, or aspirin or irradiation. In such cells, the response to TRAIL-induced apoptosis may be significantly increased. It has been suggested in the literature, that this signalling pathway is primarily mediated through DR5. Decoy receptors can also be induced by such treatments indicating that selective targeting of DR5 would achieve more efficient tumor cell killing.

The inventors have discovered that the TRAIL variants of the present invention are particularly useful for treating cancers of the gastrointestinal and gynaecological systems.

Gynaecological cancers include but are not limited to cancers of the ovaries, cervix, uterus and vagina. Further gynaecological cancers will be known to those of skill in the art.

The TRAIL variants of the present invention may also be useful to treat hormone dependent cancers which include but are not limited to cancers of the thyroid, prostate and breast. Other hormone-dependent cancers are known to those of skill in the art. Since the growth of such cancers is stimulated by particular hormones, it is possible to combine the treatment with the TRAIL variant, or the pharmaceutical composition comprising a TRAIL variant, with hormone therapies.

In a preferred embodiment of the invention, the TRAIL variant, or the pharmaceutical composition comprising a TRAIL variant, is used to treat ovarian cancer. The inventors have discovered that the use of the TRAIL variants, of the present invention, results in increased apoptosis of the ovarian cancer cells in vitro and in vivo, when compared to wtTRAIL.

Different types of ovarian cancer are known. There are several types of ovarian malignancies, each with its own histopathologic appearance and biologic behaviour. The World Health Organization (WHO) classifies ovarian tumors based on their cell type: epithelial, germ cell, and stromal whereby epithelial tumors are by far the most common type. We have found that primary ovarian epithelial tumors are thereby particularly suitable for treatment with the TRAIL variants of the present invention since they express high levels of the DR5 receptor. Furthermore we show that chemotherapy and treatment with aspirin respectively can further upregulate DR5 receptor expression in these cells.

The inventors have discovered that the D269HE195R TRAIL variant increases apoptosis by at least 2 fold in the ovarian cancer cell line A2780. The inventors have also discovered that the D269HE195R and D269H TRAIL variants increase apoptosis by 2.7-4.2 fold in the colon adenocarcinoma cell line Colo205. Furthermore, studies on an IP xenograft ovarian cancer mouse model have revealed that variant TRAIL protein (D269HE195R) is more effective than wtTRAIL in inducing apoptosis in vivo. In this model system the cancerous cells can be detected by luminescence and the treatment with the mutant TRAIL protein was found to result in higher mean signal reduction (68.3%) compared to wtTRAIL (48.8%) which indicates that the TRAIL variant demonstrated greater efficiency in decreasing the size of the ovarian tumor.

By the terms “increase apoptosis” and “enhanced apoptosis induction” it is meant that the TRAIL variants of the present invention increase the number of cell that undergo apoptosis, when compared to a sample which was treated with wtTRAIL or other suitable controls, which will be evident to those skilled in the art.

The progression of cancer is monitored by a staging process. This indicates how well developed the cancer is and if it has spread. Ovarian cancer is thereby usually classified according to the system established by the International Federation of Gynecology and Obstetrics (FIGO). While the example below shows the FIGO system for ovarian cancer, the classification of the various cancer stages is very similar for other gynaecological malignancies. The score runs from one to four, with the prognosis becoming progressively worse at each stage.

### Stage I—Malignant Cells Limited To One Or Both Ovaries

IA—involves one ovary; capsule intact; no tumor on ovarian surface; no malignant cells in ascites or peritoneal washings

IB—involves both ovaries; capsule intact; no tumor on ovarian surface; negative washings

IC—tumor limited to ovaries with any of the following: capsule ruptured, tumor on ovarian surface, positive washings

### Stage II—Pelvic Extension Or Implants

IIA—extension or implants onto uterus or fallopian tube; negative washings

IIB—extension or implants onto other pelvic structures; negative washings

IIC—pelvic extension or implants with positive peritoneal washings

### Stage III—Microscopic Peritoneal Implants Outside of the Pelvis; Or Limited To the Pelvis With Extension To the Small Bowel Or Omentum

IIIA—microscopic peritoneal metastases beyond pelvis

IIIB—macroscopic peritoneal metastases beyond pelvis less than 2 cm in size

IIIC—peritoneal metastases beyond pelvis >2 cm or lymph node metastases

### Stage IV—Distant Metastases

The treatment of metastatic tumors, either at initial presentation, progression on therapy or relapse following initial adjuvant therapy in primary tumors derived from the gynaecological system, also forms one embodiment of the present invention. Metastatic tumors may be derived from a primary tumor which is an ovarian tumor.

In a further embodiment of the invention the TRAIL variant, or the pharmaceutical composition comprising the TRAIL variant, are used to treat cancers of the gastrointestinal system. These include cancers of the esophagus, stomach, liver, biliary system, pancreas, bowels, and rectum. The inventors have surprisingly discovered that the use of the DR5-selective TRAIL variant of the invention results in higher apoptosis in colon cancer cells, compared to cells which were treated with wtTRAIL.

Again, gastrointestinal cancers are not a single disease and can be classified according to the cell type which include, but are not limited to: adenocarcinoma, leiomyosarcoma, lymphoma and neuroendocrine tumors, with adenocarcinomas of the colon being the most common type. We have found that primary colon cancer cells, heriditary non-polyposis colon cancer (HNPCC) and familial adenomatous polyposis (FAP) are thereby particularly suitable for treatment with the TRAIL variants of the present invention since they express high levels of the DR5 receptor. Another embodiment of the invention is the use of a TRAIL variant as primary prevention in a population of people at risk of hereditary colon cancers.

Similar to gynaecological malignancies, as described above, the progression of colon cancers can also be monitored by a staging process. For colon cancer the TNM ([primary] tumor, [regional lymph] node, [remote] metastasis) staging is usually used.

TABLE 1
TNM Staging System for Colon Cancer

| | | Regional Lymph | Remote |
|---|---|---|---|
| Stage | Primary Tumor (T) | Node (N) | Metastasis (M) |
| Stage 0 | Carcinoma in situ | N0 | M0 |
| Stage I | Tumor may invade | N0 | M0 |
| | submucosa (T1) or | | |
| | muscularis (T2). | | |
| Stage II | Tumor invades | N0 | M0 |
| | muscularis (T3) or | | |
| | perirectal tissues (T4). | | |
| Stage | T1-4 | N1 | M0 |
| IIIA | | | |
| Stage | T1-4 | N2-3 | M0 |
| IIIB | | | |
| Stage | T1-4 | N1-3 | M1 |
| IV | | | |

The treatment of metastatic tumors, either at initial presentation, progression on therapy or relapse following initial adjuvant therapy in primary tumors derived from the gastrointestinal system, is also an embodiment of the present invention. In a preferred embodiment, the metastatic tumors are derived from a primary tumor, which is a colon tumor.

Results obtained with Colo205, LoVo, SW948, ML-1, HeLa, Caski, SiHa, A2780, and BJAB cell lines and discussed herein show that the biological activity of the tested D269H, D269HE195R, and D269HT214R TRAIL variants is specifically directed towards the DRS receptor and that these TRAIL variants have a high efficiency in inducing apoptosis in colon, ovarian and cervical cancer cell lines.

A TRAIL variant according to the invention preferably exhibits superior selectivity for the death receptor 5 (TRAIL-R2) over the decoy receptors DcR1 (TRAIL-R3) and DcR2 (TRAIL-R4). DcR1 and DcR2 do not contain a death domain or contain a truncated death domain, respectively. Binding of TRAIL to these receptors does not induce apoptosis; on the contrary, it may actually prevent apoptosis by sequestering available TRAIL from DR4 and DR5, or by leading to NF-κB activation via DcR2. For this reason, it is preferred that the TRAIL variants of the invention are not sequestered via this route.

The inventors have shown that the increase in association rate constant (kₒₙ) of D269HE195R for binding to the DR5 receptor was less than 5 fold improved when compared to the kₒₙ of wtTRAIL but that kinetics of apoptosis induction in Colo205 cells surprisingly showed a 17-fold increase. To reach 50% of its total efficacy, wtTRAIL required 60.8±4.3 min of incubation whilst the time to reach the same value for D269HE195R was only 3.6±0.4 min. The inventors showed that incubation of colon adenocarcinoma cell line Colo205 with DcR1 neutralising antibody prior to treatment with wtTRAIL led to a 1.51±0.15 fold increase in apoptosis, whilst incubation with DcR1 neutralising antibody prior to treatment with D269HE195R showed no increase in apoptosis (FIG. 14D). The inventors have also shown that incubation of Colo205 cells with DcR2 neutralising antibody prior to treatment with wtTRAIL led to a modest 1.22±0.07 fold increase in apoptosis, whilst incubation with DcR2 neutralising antibody prior to treatment with D269HE195R showed no increase in apoptosis (FIG. 14D). Combining neutralizing antibodies to both DcR1 and DcR2 resulted in a further and nearly additive (1.68±0.12 fold) increase in apoptosis and a modest (1.5±0.1 fold) apoptosis rate enhancement for TRAIL while the level and rate of apoptosis for D269HE195R was not affected. Although decoy receptor neutralization increased the apoptosis-inducing potency of wtTRAIL by 1.68 fold, this increase alone did not fully explain the 3.3-4.2 fold higher and 17 fold faster pro-apoptotic activity of D269H/E195R. Since the half life of wtTRAIL is acknowledged as being approximately 30 minutes, it will be appreciated that a variant which binds effectively will overcome the problems associated with the short half-life of these recombinant proteins.

In order to account for the higher and faster pro-apoptotic activity of D269HE195R, receptor binding of TRAIL and D269HE195R on the cell surface was simulated using a mathematical model describing all possible binding events. From the simulation data it could be rationalized that the observed effect is a combination of increased affinity for DR5 and decreased affinity for DR4, DcRI and DcR2. This was indicated further when the formation of heterotrimeric complexes was studied by simulation. wtTRAIL triggered a much higher amount of heteromeric receptor complexing, especially during the first 30 min of incubation. This receptor pool appeared to be dynamically changing and gradually disappearing, due to rearrangement into homomeric complexes. D269HE195R on the other hand resulted in a much lower level of receptor hetero-trimerization and only during the first 5 min of the incubation. Given the short in vivo half-life reported for wtTRAIL,³⁸ faster receptor binding kinetics and faster induction of apoptosis of D269H and D269HE195R can result in a considerable therapeutic advantage.

The inventors demonstrated that, in order to be therapeutically fully efficient and effective, it is important to increase both the association rate constant and affinity constant (K_{d}) for the target receptor as well as to decrease the affinity constant (K_{d}) for (reduce binding to) any off-target receptor(s) for the therapeutic potency of cytokines and other proteins that show receptor binding promiscuity.

For example, this might be of particular interest in the development of agonistic or antagonistic variants of the TNF ligand family that bind multiple receptors in addition to TRAIL (e.g. TNF-α, TNF-β, FASL, RANKL, APRIL, BAFF, Light, TL1A). A variant that has to activate or block a particular receptor can be made more efficient by reducing binding to its other receptors in addition to improving binding to its target receptor. Such variants may be obtained by rational design, as demonstrated by the inventors in case of e.g. D269HE195R by employing a computational design method, or for example by using directed evolution methods and/or high throughput screening techniques.

In one aspect of the present invention, there is provided a method of enhancing therapeutic efficacy of a protein by enhancing the kinetics of activation (or blocking) of a particular target receptor by changing its receptor binding specificity. Preferably, kinetics of activation (or blocking) of the protein is enhanced by increasing the binding and binding rate of the protein to the preferred target receptor. More preferably the kinetics of activation (or blocking) of the target receptor is increased by reducing the binding of the protein to one or more off-target receptors. Most preferably, increasing the binding and binding rate of the protein to the preferred target receptor is combined with reducing the binding of the protein to one or more of its off-target receptors. Preferably proteins with improved kinetic properties are produced by directed evolution or high throughput screening. More preferably the method of improving kinetic properties involves a combination of directed evolution and high throughput screening. Preferably the method of enhancing therapeutic efficacy uses a rational design approach, and in some embodiments this approach may include computational protein design. More preferably, rational design methods may be combined with directed evolution and/or high throughput screening methods. Preferably the protein which shows enhanced therapeutic efficacy is a cytokine. More preferably the protein is a promiscuous cytokine which binds to more than one receptor. More preferably the protein is a member of the TNF ligand family. More preferably the TNF ligand is a promiscuous TNF ligand, including but not limited to TNF-α, TNF-β, FASL, TRAIL, RANKL, APRIL, BAFF, Light or TL1A. Most preferable, the protein is TRAIL.

The variant TRAIL molecules of the present invention are of great utility in inducing apoptosis in cells. By “induces apoptosis” is meant that a compound according to the present invention acts to cause cell death in target cells. Apoptosis may be induced in vivo, ex vivo or in vitro. Preferably, apoptosis is induced in cancerous cells, and not in healthy cells.

It was previously thought that decoy receptors may play a role in protecting non-transformed cells from TRAIL-induced apoptosis. However, the inventors have discovered that treatment of human dermal fibroblasts and human umbilical vein endothelial cells (HUVEC) with wtTRAIL, D269H and D269HE195R respectively did not result in a decrease in cell viability (see FIG. 15). Preferably the TRAIL variants of the invention do not affect the viability of healthy, non-transformed cells.

Apoptosis can be measured by a number of different assays, as will be clear to those of skill in the art. Examples include DNA laddering assays (see, for example, EP0835305; Immunex); detection of chromatin fragmentation and condensation with Hoechst33342, staining and detection of phosphatidylserine exposure in combination with membrane permeabilisation measured by staining with Annexin V and propidium iodide. What these assays share in common is a measurement of biochemical or morphological changes occurring in dying cells upon sustained contact with a concentration of the compound whose activity is being measured. Cell death can be expressed as an increase of the percentage of dying cells in response to exposure to the compound (i.e. the percentage of dying cells in untreated, control cell population is subtracted from that of in cell populations exposed to the drug). Effective concentrations are typically calculated in the form of IC50 values, which is the concentration of compound at which 50% of the cells undergo apoptosis. Preferably, a compound according to the invention induces apoptosis in 50% of cells at a concentration of between 1 ng/ml and 1,000 ng/ml, more preferably between 1 ng/ml and 100 ng/ml, more preferably between 1 ng/ml and 10 ng/ml, and more preferably 44 ng/ml.

Preferably, according to the invention, useful compounds possess IC₅₀ values of between 1 ng/ml and 1,000 ng/ml, more preferably between 1 ng/ml and 100 ng/ml, more preferably between 1 ng/ml and 10 ng/ml and more preferably 4 ng/ml.

Alternatively, apoptosis can also be measured by caspase activation and other assays known to those of skill in the art.

By “variant” TRAIL protein is meant that the TRAIL protein differs in at least one amino acid position from the wtTRAIL protein (also known as TNFSF10, TL2; APO2L; CD253; Apo-2L), Entrez GeneID: 8743; accession number NM_{—}003810.2; UniProtKB/Swiss-Prot: P50591; UniProtKB/TrEMBL: Q6IBA9.

By “selectivity” is meant that the variants of the invention have substantially greater affinity for DR5 than their affinity for the decoy receptors DcR1 (TRAIL-R3) and DcR2 (TRAIL-R4) in relative terms, and preferably also for DR4. By “substantially greater affinity” we mean that there is a measurably higher affinity of the TRAIL variant for DR5 as compared with its affinity for DR4, DcR1 and DcR2. Preferably, the affinity is at least 1.5-fold, 2-fold, 5-fold, 10-fold, 100-fold, or even 1,000-fold or greater for DR5 than for one or more of DR4, DcR1 and DcR2. More preferably, the affinity is at least 1.5-fold, 2-fold, 5-fold, 10-fold, 100-fold, or even 1,000-fold or greater for DR5 than for at least two, preferably all of DR4, DcR1 and DcR2. Methods for measuring the binding affinity of proteins for binding partners are well known in the art, including for example, competition assays, Surface Plasmon Resonance and so on.

Preferably, the binding affinity of the TRAIL variants of the invention for DR5 is also higher in absolute terms than the binding affinity of the wtTRAIL molecule for DR5. Furthermore, the binding affinity of the TRAIL variants of the invention for DR4 should be lower than the binding affinity of the wtTRAIL molecule for DR4. The same is preferably true of the binding affinity demonstrated for DcR1 and DcR2.

Preferably, a TRAIL variant according to the invention displays a binding affinity of less than Kd=1,000 nM for DR5, preferably less than Kd=100 nM, more preferably less than Kd=10 nM, more preferably less than Kd=5 nM, more preferably less than Kd=1 nM, measured by surface plasmon resonance as described before where the affinity of wtTRAIL was measured to be 7.2 nM.

Preferably, a TRAIL variant according to the invention displays a binding affinity of more than Kd=1 nM for DR4, preferably more than Kd=5 nM, preferably more than Kd=10 nM, preferably more than Kd=100 nM, more preferably more than Kd=1,000 nM measured by surface plasmon resonance as described before¹⁰ where the affinity of wt TRAIL was measured to be 2.5 nM.

Preferably, a TRAIL variant according to the invention displays a binding affinity of more than Kd=10 nM for DcR1, preferably more than Kd=25 nM, preferably more than Kd=50 nM, preferably more than Kd=100 nM, more preferably more than Kd=1,000 nM measured by surface plasmon resonance as described before¹⁰ where the affinity of wtTRAIL was measured to be about 25 nM.

Preferably, a TRAIL variant according to the invention displays a binding affinity of more than Kd=10 nM for DcR2, preferably more than Kd=25 nM, preferably more than Kd=50 nM, preferably more than Kd=100 nM, more preferably more than Kd=1,000 nM measured by surface plasmon resonance as described before.¹⁰ where the affinity of wtTRAIL was measured 10 to be about 8 nM.

Preferably DR5 receptor activation by D269HE195R occurs at least 2 fold faster than with wtTRAIL, preferably at least 5 fold faster than with wtTRAIL, more preferably at least 10 fold faster than with wtTRAIL, more preferably at least 15 fold faster than with wtTRAIL, more preferably at least 17 fold faster than with wtTRAIL, and more preferably at least 20 fold faster than with wtTRAIL.

Preferably induction of apoptosis by D269HE195R occurs at least 2 fold faster than with wtTRAIL, preferably at least 5 fold faster than with wtTRAIL, more preferably at least 10 fold faster than with wtTRAIL, more preferably at least 15 fold faster than with wtTRAIL, more preferably at least 17 fold faster than with wtTRAIL, and more preferably at least 20 fold faster than with wtTRAIL.

It is preferred that the TRAIL variant of the present invention contains the mutation D269H. All references to amino acid positions in the TRAIL protein sequence presented herein, and to specific TRAIL mutants are intended to refer to the amino acid sequence given in SEQ ID NO:1. This mutant has a highly reduced binding affinity to DR4 and an increased affinity for the DR5 receptor. Further details of the binding properties of this mutant can be found in co-pending international patent application WO05/056596.

This mutant further has increased ability to induce apoptosis in various cancer cell lines. For example, it has been shown to induce apoptosis in the colon carcinoma cell Colo205 with around 4 times more efficiency compared to wtTRAIL. Furthermore the inventors have discovered that the mutant also has 3-4 times higher efficiency in inducing apoptosis in ovarian cancer cells. It is also preferred that the D269H TRAIL variant of the present invention further contains the mutations E195R and/or T214R. These mutants show superior selectivity for DR5 and decreased binding to DR4, when compared to wtTRAIL. The binding properties of these mutants have already been discussed in WO05/056596.

In one preferred embodiment of the invention the mutant is D269HT214R. This mutant has been shown to increase the apoptosis rate in the colon cancer cell line Colo205 by 1.5 fold compared to wtTRAIL at a lower concentration (see FIG. 8C). Furthermore, it also shows 2 times improved efficiency in inducing apoptosis in the ovarian cancer cell line A2780.

In a further preferred embodiment of the invention the mutant is D269HE195R. The inventors have discovered that this mutant increases apoptosis by 3-4 fold in the ovarian cancer cell line A2780. Furthermore, studies on an IP xenograft ovarian cancer mouse model have surprisingly revealed that this mutant TRAIL protein is more effective than wtTRAIL in inducing apoptosis in vivo. In this model system the cancerous cells can be detected by luminescence and the treatment with the mutant TRAIL protein resulted in higher mean signal reduction (68.3%) compared to wtTRAIL (48.8%) which indicates that the TRAIL variant demonstrated greater efficiency in decreasing the size of the ovarian tumor. Furthermore, the mutant was able to increase the level of apoptosis in the colon cancer cell line Colo205 by 2.7-4.2 fold, and an enhanced ability to induce apoptosis in colon carcinoma cell lines LoVo and SW948 and cervical carcinoma cell lines HeLa, Caski and SiHa.

The above mutations may be introduced into the full length TRAIL sequence. Preferably, however, the above mutations are introduced into soluble forms of the TRAIL sequence, such as forms comprising amino acids 114-281 or comprising amino acids 95-281; other examples will be clear to those of skill in the art. Preferred TRAIL variants according to.the invention are thus variants of the soluble fragments of the full length TRAIL sequence given in SEQ ID NO:1.

A preferred soluble fragment template comprises amino acids 114-281 (herein termed TRAIL) and all mutants described herein are of this length. The wtTRAIL sequence of TRAIL (114-281) preceded by a methionine is presented in SEQ ID NO: 3 and a preferred coding sequence is presented in SEQ ID NO:4; variants of the invention may thus be derived from this sequence.

However, as the skilled reader will appreciate, variations in these soluble templates will very likely retain the properties of this soluble form and show biological activity if additional residues C terminal and/or N terminal of these boundaries in the polypeptide sequence are included. For example, an additional 1, 2, 3, 4, 5, 10, 20 or even 30 or more amino acid residues from the wild-type TRAIL sequence, or from a homologous sequence, may be included at either or both the C terminal and/or N terminal of these boundaries, without prejudicing the ability of the polypeptide fragment to fold correctly and exhibit biological activity. Similarly, truncated variants of this template in which one or a few amino acid residues (for example, 1, 2, 3, 4, 5, 10 or more) may be deleted at either or both the C terminus or the N terminus without prejudicing biological activity.

The invention thus provides DR5-specific TRAIL variants for use as a medicament. The invention also provides a method for treating a subject suffering from or at risk of contracting a disease, comprising administering to the subject a pharmaceutical composition of the invention. The invention also provides the use of a pharmaceutical composition of the invention in the manufacture of a medicament for treating a subject. Particularly suitable diseases include cancer diseases, such as leukaemia, lymphoma, melanoma, prostate, pancreatic, bladder, kidney, head and neck, liver and breast cancer, cancers of the lung, ovaries, cervix, colon, and multiple myeloma.

In a preferred embodiment the cancer is ovarian cancer or colon cancer. In another embodiment, the cancer may be cervical carcinoma. These are among the most common cancers in the world and are also a leading cause of cancer-related deaths. Both ovarian and colon cancer are usually only discovered at late stages, due to the absence of obvious early warning signs. Furthermore, it is often observed that the initial response to treatment is good in advanced stage patients, but the overall survival rate is low due to the occurrence of drug resistance. It is therefore of paramount importance to improve the treatment of these diseases.

The TRAIL variant, which is administered to the mammal diagnosed with cancer, should be present at a therapeutically effective amount e.g. an amount sufficient to induce apoptosis. The precise effective amount for a given patient will depend upon their size and health, the nature and extent of the disease, and the composition or combination of compositions selected for administration. The effective amount can be determined by routine experimentation and is within the judgement of the clinician. For purposes of the present invention, an effective dose will generally be from about 0.01 mg/kg to about 5 mg/kg, or about 0.01 mg/kg to about 50mg/kg or about 0.05 mg/kg to about 10 mg/kg, preferably about 10 mg/kg. A suitable dose should be used so as to achieve a serum concentration in the patient of between 0.1 and 1,000 ng/ml, preferably between 1 ng/ml and around 100 ng/ml, more preferably around 10-100 ng/ml. TRAIL variants may be administered in the form of salts and/or esters.

A TRAIL variant of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature TRAIL variant may be fused with another compound, such as a compound to increase the half-life of the TRAIL variant (for example, polyethylene glycol).

These fusion proteins can be obtained by cloning a polynucleotide encoding a TRAIL variant in frame to the coding sequences for a heterologous protein sequence.

The term “heterologous”, when used herein, is intended to designate any polypeptide other than a TRAIL variant according to the invention. Examples of heterologous sequences, that can be comprised in the fusion proteins either at the N- or C-terminus, include: extracellular domains of membrane-bound protein, immunoglobulin constant regions (Fc regions), multimerization domains, domains of extracellular proteins, signal sequences, export sequences, and sequences allowing purification by affinity chromatography.

Many of these heterologous sequences are commercially available in expression plasmids since these sequences are commonly included in fusion proteins in order to provide additional properties without significantly impairing the specific biological activity of the protein fused to them¹¹. Examples of such additional properties are a longer lasting half-life in body fluids, the extracellular localization, or an easier purification procedure as allowed by the a stretch of Histidines forming the so-called “histidine tag”¹² or by the “HA” tag, an epitope derived from the influenza hemagglutinin protein¹³. If needed, the heterologous sequence can be eliminated by a proteolytic cleavage, for example by inserting a proteolytic cleavage site between the protein and the heterologous sequence, and exposing the purified fusion protein to the appropriate protease.

These features are of particular importance for the fusion proteins since they facilitate their production and use in the preparation of pharmaceutical compositions. For example, the TRAIL variant may be purified by means of a hexa-histidine peptide fused at the N-terminus or C-terminus. The hexa-histidine peptide fused to TRAIL may be eliminated by proteolytic cleavage, as it is know that such a hexa-histidine peptide fusion renders TRAIL more toxic towards non-cancerous cells. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the sequence of the substances of the invention and the immunoglobulin sequence. The resulting fusion protein has improved properties, such as an extended residence time in body fluids (i.e. an increased half-life), increased specific activity, increased expression level, or the purification of the fusion protein is facilitated.

The TRAIL variant of the invention may also be fused with a marker protein, for example a fluorescent protein, like green fluorescent protein (GFP), yellow fluorescent protein (YFP) and similar proteins. Such a protein is particularly advantageous for diagnostic purposes.

In one embodiment, the protein is fused to the constant region of an Ig molecule. Examples of Ig molecules include heavy chain regions, like the CH2 and CH3 domains of human IgG1. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

In a further preferred embodiment, the TRAIL variant may comprise at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

To facilitate biological synthesis of TRAIL variants, one aspect of the invention provides nucleic acid molecules that encode the TRAIL variants of the invention. The coding sequence for wild type TRAIL is given in accession number NM_{—}003810. Nucleic acid molecules coding for TRAIL variants according to the invention may be derived from this sequence by supplementing the appropriate coding sequence at the mutation point(s). Examples of preferred nucleic acid molecules according to the invention are variants of the sequences presented in SEQ ID NO: 2 (full length gene); or nucleotides 88-933 (846 nucleotides long) of SEQ ID NO:2 which is the coding sequence. A preferred coding sequence for wild type TRAIL (amino acids 114-281) is presented in SEQ ID NO:4 (TRAIL 114-281 preceded by methionine) and so the variants of the present invention are preferably encoded by variants of this sequence.

In order to introduce mutation(s) into the full length or TRAIL coding sequence, the skilled person will be perfectly able to substitute the necessary codon at the relevant position in the sequence. All amino acid numbers referred to herein relate to the full length TRAIL protein sequence. To account for codon bias between different host organisms, the skilled person may refer to published texts on this matter or common general knowledge.

For example, codon usage in various different species can be found at: littp://www.kazusa.or.jp/codon/; in particular for Escherichia coli codon usage information can be found at: http://www.kazusa.or.jp/codon/cgi-bin/showcodon.cgi?species=155864.

The nucleic acid may be DNA or RNA (or hybrids thereof), or their analogues, such as those containing modified backbones (e.g. phosphorothioates) or peptide nucleic acids (PNA). It may be single stranded (e.g. mRNA) or double stranded, and the invention includes both individual strands of a double-stranded nucleic acid (e.g. for antisense, priming or probing purposes). It may be linear or circular. It may be labelled. It may be attached to a solid support.

Nucleic acids, according to the invention can, of course, be prepared in many ways e.g. by chemical synthesis (e.g. phosphoramidite synthesis of DNA) in whole or in part, by nuclease digestion of longer molecules, by ligation of shorter molecules, from genomic or cDNA libraries, by use of polymerases etc.

Accordingly, the present invention also provides vectors (e.g. plasmids) comprising nucleic acids of the invention (e.g. expression vectors and cloning vectors) and host cells (prokaryotic or eukaryotic) transformed with such vectors.

The invention also provides a process for producing a TRAIL variant of the invention, comprising the step of culturing a host cell transformed with nucleic acid of the invention under conditions that induce expression of the variant.

Suitable expression systems for use in the present invention are well known to those of skill in the art and many are described in detail in Sambrook (1989)¹⁴ and Fernandez et al. (1998)¹⁵. Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook¹⁴. Generally, the encoding gene can be placed under the control of a control element such as a promoter, ribosome binding site (for bacterial expression) and, optionally, an operator, so that the DNA sequence encoding the desired peptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the peptides of the invention.

For long-term, high-yield production of a recombinant polypeptide, stable expression is preferred. For example, cell lines that stably express the peptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers et al.¹⁶. Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; U.S. Pat. No. 5,608,143 and Zenk (1991)¹⁷. In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred prokaryotic expression systems include those that use streptococci, staphylococci, E. coli, Streptomyces and Bacillus subtilis as host cells.

Examples of particularly suitable fungal expression systems include those that use yeast (for example, S. cerevisiae) and Aspergillus as host cells.

In one embodiment of the invention, the TRAIL variant may be used to remove cancerous cells from a patient's body fluids. In one aspect, the TRAIL variant is used to contact a patient's blood ex vivo and thereby remove cancerous cells from the blood, since they bind to the DR5-specific TRAIL variant with higher affinity than non-cancerous cells. The blood can then be reintroduced into the patient. It is also possible to aspirate bone marrow from a patient and contact the bone marrow with the DR5-specific TRAIL variant, whereby the cancerous cells which have a higher affinity for the TRAIL variant compared to non-cancerous cells, are removed from the bone marrow. The bone marrow can then be reintroduced into the patient. Whenever a body fluid is contacted ex vivo with the TRAIL variant of the invention it is preferred that the TRAIL variant is immobilized on a suitable matrix. Further uses will be apparent to the person skilled in the art.

Preferably, suitable cancers, which can be treated with the TRAIL variant of the invention, include cells that express the DR5 receptor on the surface as measured by flow cytometry or by immunohistochemistry (IHC) of primary tumor samples. Such cancer cells are readily identifiable by various means known to those of skill in the art which include, but are not limited to, immunocytochemistry with receptor specific antibodies, Fluorescent-activated cell sorting (FACS) with receptor specific antibodies, western blot analysis with receptor specific antibodies etc. DR4 specific antibodies can be obtained, for example, from Abcam (ab8414). DR5 antibodies are available as well (Sigma-Aldrich, D3938).

In a preferred embodiment, the expression of the DR5 receptor on the cell surface is higher than the expression of the DR4 receptor. The expression levels of the protein can thereby be assessed by various techniques, known to those of skill in the art, which include, but are not limited to, quantitative western blot analysis, FACS with fluorescently labelled DR5 and/or DR4 specific antibodies and others. “Higher expression” and “upregulation” mean that the protein expression is increased 1.5 fold (more preferably, 2 fold, 4 fold, 8 fold, 16 fold, 32 fold, 100 fold or even 1,000 fold) relative to another protein. Accordingly, “lower expression” means that the protein expression is reduced 1.5 fold (more preferably, 2 fold, 4 fold, 8 fold, 16 fold, 32 fold, 100 fold or even 1,000 fold) relative to another protein.

In a more preferred embodiment the DR4 receptor is not expressed on the cell surface at any detectable level, whereas the DR5 receptor is expressed. “No expression” thereby means that the receptor is not detectable with the techniques discussed above and other suitable techniques, known to those of skill in the art. In another embodiment the DR5 receptor is expressed at similar levels as DcR1 and/or DcR2. In a more preferred embodiment the DR5 receptor is expressed more highly compared to DcR1 and/or DcR2. In an even more preferred embodiment, the cancerous cell expresses the decoy receptors DcR1 and DcR2 more highly in response to a chemotherapeutic agent. Such cells are preferred because wtTRAIL shows reduced efficiency in inducing apoptosis in these cells due to binding to the decoy receptor and sequestering of wtTRAIL. Therefore, the DR5-specific TRAIL variant of the present invention would be particularly advantageous.

In one preferred embodiment the cancer cell upregulates the DR5 receptor expression in response to a chemotherapeutic agent. In another preferred embodiment the cancer cell upregulates the DR5 receptor expression in response to aspirin. The inventors are of the opinion that receptors DR5 and/or DR4 may be up-regulated after treatment with DNA damaging chemotherapeutic drugs or aspirin. In such cells, the response to TRAIL-induced apoptosis may be significantly increased. It has been suggested in the literature, that this signalling pathway mainly goes through DR5.

TRAIL variants of the invention may be co-administered with one or more other compounds, preferably antitumor compounds, more preferably those which are active against the cancerous cells targeted by the variants of the invention or those which increase responsiveness of the tumor to the TRAIL variants. Compositions of the invention may thus include one or more antitumor agents, examples of which will be known to those of skill in the art and include gamma irradiation as well as chemotherapeutic drugs such as alkylating agents, anti-metabolites, plant alkaloids and terpenoids, vinca alkaloids, podophyllotoxin derivatives, taxanes, topoisomerase inhibitors, antitumor antibiotics, monoclonal antibodies, DNA damaging drugs, histone deacetylase inhibitors, hormones, proteasome inhibitors and so on. In a preferred embodiment the chemotherapeutic agent is an anti-angiogenesis antibody such as bevacizumab, a proteasome inhibitor such as Bortezomib or a DNA damaging drug such as 5-Flurouracil. Preferably, the chemotherapeutic agent used acts to increase the surface expression of the DR5 (TRAIL-R2) receptor on the cancerous cells and/or enhance/potentiate apoptosis-induction through DR5. In further embodiments, compositions of the invention may include aspirin.

Antitumor compounds act in a variety of ways, and each disrupt the tumor at different targeted sites when used in combination. It is therefore likely that the TRAIL variants of the invention will work well in combination with agents that target multiple oncogenic pathways such as the multiple tyrosine kinase inhibitors, including but not limited to sorafenib and sunitnib, as well as other similar agents currently in development. Pre-clinical trials have shown enhanced effected of TRAIL when used in combination with such agents (data not shown).

By “enhanced apoptosis induction” it is meant that the chemotherapeutic agent increases the number of cells that undergo apoptosis in the presence of the chemotherapeutic agent when compared to a sample which was not exposed to the chemotherapeutic agent. Preferably the increase is 1.5 fold (more preferably 2 fold, 4 fold, 8 fold, 10 fold, 20 fold, 100 fold or even 1,000 fold).

By “enhanced apoptosis induction” it is meant that aspirin increases the number of cells that undergo apoptosis in the presence of aspirin when compared to a sample which was not exposed to the chemotherapeutic agent. Preferably the increase is 1.5 fold (more preferably 2 fold, 4 fold, 8 fold, I Ofold, 20 fold, 100 fold or even 1,000 fold).

Such cancerous cells can be identified by various means known to those of skill in the art. For example, a fluorescently labelled DR5 and/or DR4 antibody can be used to decorate the cell, which is to be tested. The expression of DR5 and DR4 can then be evaluated by fluorescent activated cell sorting (FACS) before and after exposure to a chemotherapeutic agent or aspirin.

Preferred cancer cells, for treatment with the DR5-specific TRAIL variant of the invention, are those which upregulate the DR5 receptor 1.5 fold (more preferably, 2 fold, 4 fold, 8 fold, 16 fold, 32 fold, 100 fold or even 1,000 fold) compared to the control. Other means to assess the upregulation of the DR5 receptor, in response to a chemotherapeutic agent, are known to those of skill in the art.

In a preferred embodiment of the invention the chemotherapeutic agent is cisplatin (also known as cisplatinum or cis-diamminedichloroplatinum (II) (CDDP)). The inventors have shown that exposure of A2780 cells to cisplatin increased the expression of the DR5 and DcR2 receptor on the cell (FIG. 1A). Furthermore, it is demonstrated herein that the administration of cisplatin, in combination with the TRAIL variants of the invention, results in higher apoptosis in cancerous cells in vitro (FIGS. 2 and 3) and in vivo (FIG. 6) and increased survival rates in mouse xenograft models (FIG. 6C). Therefore it may be preferred to administer the TRAIL variant of the invention in conjunction with cisplatin or other platinum agents such as carboplatin or oxaliplatin. The effective amount to be administered to a patient can be determined by routine experimentation and is within the judgement of the clinician. For purposes of the present invention, an effective dose will generally be from about 0.1 ng/ml to about 1,000 ng/ml, or about 1 ng/ml to about 100 ng/ml or about 10 ng/ml to about 100 ng/ml, in the blood.

The inventors have also discovered that mice suffering from colon carcinoma induced by inoculation with Colo205 cells show an increased reduction in tumor growth when treated wtTRAIL or D269HE195R respectively (FIG. 25).

In one embodiment of the invention, the TRAIL variant may be used to screen for cancerous cells, which are likely to benefit from treatment with the TRAIL variant of the invention. The TRAIL variant can be exposed to the cells, which are to be tested. These cells can be either cell lines derived from a tumor or samples obtained after a tissue biopsy. Cells which are likely to benefit from treatment with the TRAIL variant of the invention are those to which the TRAIL variant can bind. This can be assessed, for example, by receptor binding assays. Alternatively, it is also envisioned that the recombinant TRAIL variant is either fluorescently labelled post-translationally or expressed as a fusion protein with a fluorescent protein, such as GFP etc. Cells to which the thereby labelled TRAIL variant binds are then easily identifiable by various means known to those of skill in the art. For example, the fluorescent TRAIL variant bound to the cell can be visualised by fluorescent microscopy. It is also possible to use FACS to assess binding of TRAIL. In all cases the results have to be compared to a control, for example an identical experiment performed with unlabelled TRAIL. In a preferred embodiment, the cell line which can be identified by the means described above, upregulates the DR5 receptor in response to a chemotherapeutic agent or aspirin. Such an upregulation can be assessed by comparing the binding of the variant TRAIL protein before and after exposure to the chemotherapeutic agent or aspirin.

Other suitable cancers are those which show an increased apoptosis rate in response to the TRAIL variants of the present invention. In particular, primary tumor cells and cell lines which show increased apoptosis in the presence of the TRAIL variants of the present invention when compared to wtTRAIL or other suitable controls, that will be evident to those skilled in the art, are a preferred embodiment of the present invention.

A further aspect of the invention may comprise a pharmaceutical composition comprising a mutant cytokine, nucleic acid or vector as described above, in conjunction with a pharmaceutically-acceptable carrier. The invention provides a pharmaceutical composition comprising (a) TRAIL variant(s), nucleic acid or vector as described above and (b) a pharmaceutical carrier.

Component (a) is the active ingredient in the composition, and this is present at a therapeutically effective amount e.g. an amount sufficient to induce apoptosis. The precise effective amount for a given patient will depend upon their size and health, the nature and extent of the disease, and the composition or combination of compositions selected for administration. The effective amount can be determined by routine experimentation and is within the judgement of the clinician. For purposes of the present invention, a suitable dose should be used so as to achieve a serum concentration of 0.1 and 1,000 ng/ml, preferably between 1 ng/ml and around 100 ng/ml, more preferably around 10-100 ng/ml. TRAIL variants may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in Gennaro¹⁸.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe.

In certain embodiments of the invention, as described above, it is preferred that a compound according to the invention is administered in conjunction with another agent, such as an anti-tumor agent. Suitable examples of such agents for use in combination with the pharmaceutical composition of the present invention are known in the art and examples are listed above.

It is also envisioned that a pharmaceutical composition, comprising the variant TRAIL of the invention, may on occasion be administered in conjunction with antibodies against one or more DR4, DcR1 or DcR1. In such a manner it is possible further to block signalling through the DR4 specific pathway or binding of the TRAIL variant to decoy receptors. This is advantageous since any residual binding activity to receptors other than DR5 is inhibited and specificity of the mutant cytokine of the invention is enhanced even further.

Compositions of the invention will generally be administered directly to a subject. Direct delivery may be accomplished by parenteral injection (e.g. subcutaneously, intraperitoneally, intravenously, intramuscularly, or to the interstitial space of a tissue; also by direct injection into the tumor).

It is a preferred embodiment of the invention that the TRAIL variant, or the pharmaceutical composition comprising the TRAIL variant, is administered intraperitoneally. The rationale behind IP drug administration is to increase local drug exposure, while lowering plasma clearance. The inventors have discovered that injection of TRAIL by this route results in increased tumor uptake compared to intravenous injection, increased efficacy and a reduced clearance rate. Furthermore, IP injection provides reduced system toxicity compared to intravenous administration (Table 2, FIG. 3).

The inventors have also shown that specific tumor retention of ¹²⁵I-TRAIL takes place. It was demonstrated by maximal tumor activity at 60 min after IV administration, while activity in all other well perfused organs is maximal at 15 min. IP administration resulted in even higher activity in the tumor and a higher cumulative tumor to blood ratio, which shows that intraperitoneal administration of recombinant human TRAIL (herein TRAIL) results in higher tumor drug exposure compared to IV administration. Moreover, 6 h after IP injection of a low dose of TRAIL, cleaved caspase-3 was detected in the superficial layers of the tumors, suggestive of TRAIL penetration by free-surface diffusion. This is a potential advantage of TRAIL and TRAIL variants over monoclonal antibodies, which show limited IP tumor penetration after IP administration. Another advantage which TRAIL and TRAIL variants possess over monoclonal antibodies against DR4/DR5 is a reduced immunogenicity. In addition, it will be more expensive to develop effective monoclonal antibodies, and the use of TRAIL and TRAIL variants is therefore more cost effective.

Dosage treatment can be a single dose schedule or a multiple dose schedule. In a preferred embodiment of the invention, the TRAIL variant or the pharmaceutical composition, comprising the TRAIL variant, is administered on a weekly basis to the patient. It is understood that the dosing regime is within the judgement of the clinician and can be altered if the need arises or if another dosage regime is found to be more beneficial for a patient.

Various aspects and embodiments of the present invention will now be described in more detail by way of example. It will be appreciated that modification of detail may be made without departing from the scope of the invention.

### BRIEF DESCRIPTION OF DRAWINGS

FIG. 1. DR5-selective variant D269H/E195R show enhance cytotoxicity compared to rhTRAIL alone and in combination with cisplatin in A2780 cells

A. Levels of TRAIL receptor membrane expression in A2780 before and after exposure to 2.5 μM of cisplatin determined by FACS analysis. Receptor expression is expressed as the fluorescence intensity (PE).

B. Survival of A2780 assessed with a cytotoxicity assay after 96 h exposure to 0-100 ng/ml TRAIL and TRAIL-DR5.*p=0.008

C. Survival of A2780 determined with a cytotoxicity assay. Cells were pre-incubated for 4 h with 2.5 μLM of cisplatin, after which the cells were washed and exposed to 0-25 ng/ml TRAIL or TRAIL-DRS for 92 h.*TRAIL vs cisplatin and TRAIL p<0.01, TRAIL-DR5 vs. cisplatin and TRAIL-DR5 p<0.01, cisplatin and TRAIL vs cisplatin and TRAIL-DR5 p<0.001.

Data represent the meant SD of at least three independent experiments.

FIG. 2. DR5-selective variant D269H/E195R show enhance apoptosis in combination with cisplatin in A2780 cells in a solely DR5-dependent manner.

A. Induction of apoptosis after exposure to 50 ng/ml TRAIL or TRAIL-DR5 alone or after pre-incubation with 2.5 μM cisplatin for 4 h, 20 h prior to TRAIL or TRAIL-DR5 administration. Caspase activation was determined with a caspase-3 activity assay after 1, 3 and 5 h of treatment.

B. Cells were pre-incubated with medium; 2.5, 10, 30 μM of cisplatin for 4 h and then washed. After 20 h, the cells were exposed for 4 h to 100 or 250 ng/ml TRAIL or TRAIL-DR5, and apoptosis was assessed by means of acridine orange staining.

C. Acridine orange apoptosis assay. The cells were pre-incubated with 2.5, 10 or 30 μM of cisplatin for 4 h and then washed. After 20 h, the cells were exposed for 1 h to 2.5 μg/ml anti-DcR2 antibody or medium, after which the cells were exposed to 100 or 250 nghnl TRAIL for 4 h. Anti-DcR2 antibody co-incubation did not cause significant changes in apoptosis induction. Data represent the mean±SD of at least three independent experiments.

D. DR5 membrane expression in A2780 cells transfected with siRNA against luciferase (Luc siRNA) or siRNA against DR5 (DRS siRNA). Cells were pre-incubated with cisplatin for 4 h, washed, cultured in media for 20 h and DR5 membrane expression was determined with flow cytometry. The mean fluorescence intensities (MFI) were corrected for staining with a non-specific isotype control.

E. DR5 cellular protein expression in A2780 cells transfected with siRNA against luciferase or siRNA against DR5 (DR5 siRNA)siRNA against DR5 (DR5 siRNA). Cells were pre-incubated with cisplatin for 4 h, washed, cultured in media for 20 h and DR5 protein expression was determined with western blotting using actin as a control for protein loading.

F. A2780 cells transfected with siRNA against luciferase (Luc siRNA) or siRNA against DR5 (DR5 siRNA) were pre-incubated with medium or 30 μM of cisplatin for 4 h and then washed. After 20 h, the cells were exposed for 4 h to 100 ng/ml TRAIL or TRAIL-DR5, and apoptosis was assessed by means of acridine orange staining. Data represent the mean E SD of three independent experiments.

Table 2.

A. Biodistribution of IV administered ¹²⁵I-TRAIL and tumor to blood ratio in mice bearing IP A2780 xenografts at 15, 30, 60, 90 and 360 min after injection. Data are expressed as % ID/g±SEM. Significant differences in activity between IV and IP: (a) p=0.015; (b) p=0.009; (c) p=0.0015; (d) p=0.025; (e) p=0.0028; (f) p=0.035.

B. Biodistribution of IP administered ¹²⁵I-TRAIL and tumor to blood ratio in mice bearing IP A2780 xenografts at 15, 30, 60, 90 and 360 min after injection. Data are expressed as % ID/g±SEM.

FIG. 3. ¹²⁵I-TRAIL biodistribution in tumor bearing mice.

A. Area under the blood activity versus curves for IP and IV ¹²⁵I-TRAIL. The blood activity was determined at 15, 30, 60, 90 and 360 min after administration of ¹²⁵I-TRAIL. The % ID/g were calculated and averaged for 3-5 mice per time point and pharmacokinetic profiles were fitted with a two-compartment model.

B. Tumor-to-blood ratio versus time of ¹²⁵I-TRAIL administered intravenously or intraperitoneally. Tumor to blood ratios were calculated by dividing the average tumor activity in %ID/g per time point through the average blood activity in % ID/g per time point.

FIG. 4. Assessment of caspase-3 activity in tumors at 15 min.

Ovarian cancer xenograft tissue excised at 15 min after IP (A) and IV (B) administration of ¹²⁵I-TRAIL, stained for cleaved caspase-3. To determine whether the higher tumor uptake after IP administration resulted in enhanced efficacy of ¹²⁵I-TRAIL, paraffin embedded tissue was obtained 15 min after ¹²⁵I-TRAILinjection and stained with cleaved caspase-3, showing no cleavage in samples obtained at this time point.

FIG. 5. Assessment of caspase-3 activity in tumors at 360 min.

Ovarian cancer xenograft tissue excised at 360 min after IP (A) and IV (B) administration of ¹²⁵I-TRAIL, stained for cleaved caspase-3. To determine whether the higher tumor uptake after IP administration resulted in enhanced efficacy of ¹²⁵I-TRAIL, paraffin embedded tissue was obtained 360 min after ¹²⁵I-TRAILinjection and stained with cleaved caspase-3, showing increased caspase-3 cleavage staining near the surface of the tumor and near small blood vessel with IP administration (FIG. 5A) whereas IV administration induced detectable caspase-3 activity near blood vessels but not near the tumor border (FIG. 5B).

FIG. 6. In-vivo efficacy of rhTRAIL and D269H/E195R monotherapy and in combination with cisplatin.

Visualization of response to TRAIL, TRAIL-DR5, cisplatin and to the combination of either ligand with cisplatin by means of bioluminescence imaging. Nude mice were inoculated IP with 2×10⁶ A2780-Luc cells. After 5 days treatment was initiated; cisplatin (4 mg/kg IP) or vehicle at day 5 and 12, TRAIL, TRAIL-DR5 (5 mg/kg IP) or vehicle at day 5-10 and 12-16, or a combination of TRAIL or TRAIL-DR5 with cisplatin. CP =cisplatin, TR=TRAIL/TRAIL-DR5.

A. Change in light emission (in radiance units) over time per treatment arm. Bioluminescent signals at each time point were averaged per treatment group and are represented by means±SEM. Differences at clay 16 were (4.6×10⁸±6.7×10⁷) vs. (2.3×10⁸±3.1×10⁷) p=0.097 between the vehicle group and TRAIL; vehicle vs. TRAIL-DR5 (1.4×10⁸±1.3×10⁷) p=0.015; vehicle vs. cisplatin (1.3×10⁸±2.4×10⁷) p=0.009; vehicle vs. cisplatin and TRAIL (6.7×10⁷±2.1×10⁷) p=0.003; vehicle vs. cisplatin and TRAIL-DRS (1.6×10⁷±4.6×10⁶) p=0.002.

B. Bioluminescent images at the end of treatment (day 16) of each 4 mice representative for 10 mice per experimental arm. Images are displayed and quantified in log radiance (photons/sec/cm²/sr).

C. Kaplan Meier survival analysis of all mice. A bioluminescent signal of >3.1×10⁸ was used as surrogate endpoint for survival, as described in the materials and methods.

FIG. 7. Cell surface expression of TRAIL receptors in Colo205 and ML-1 cells.

Cell surface expression of TRAIL receptors in Colo205 (a) and ML-1 (b) cells. (left) DR4 and DR5 receptor (right), DcR1 and DcR2.

FIG. 8. Biological activity of TRAIL and DR5-selective variants.

(A) Apoptosis-inducing activity of 100 ng/ml TRAIL in the presence of 1 μg/ml DR4 (aDR4), DR5 (aDR5), or DR4 and DR5 (+aDR4+aDR5) receptor-neutralizing antibodies in Colo205 and ML-1 cells. (B) Apoptosis-inducing activity in Colo205 cells of 100 ng/ml TRAIL or DR5-selective variants without the presence of neutralizing DR4 or DR5 antibodies (no AB) or in the presence of neutralizing antibody [aDR4, aDR5, or both (aDR4 aDR5)]. Shown is the cytotoxic potential (% cell death) of TRAIL or DR5-selective variants in Colo205 (C), ML-1 (D), and A2780 (E) and of 1, 10, or 100. ng/ml TRAIL (WT) or D269HE195R (DE) relative to cycloheximide control (0.33 μg/ml) in BJAB cells responsive to both DR4- and DR5-mediated cell death (BJAB^{wt}), BJAB cells deficient for DRS (BJAB^{DR5 DEF}), and BJAB cells deficient for DR5 stably transfected with DR5 (BJAB^{DR5 DEF}+DR5) (F).

Table 3.

EC₅₀ values of Colo205 and A2780 cells

FIG. 9. DR5 knock-down reduces apoptosis of TRAIL in combination with cisplatin in A2780 cells.

A) Cell surface expression of the DR5 receptor in A2780 DR5 knock-down and control cells in response to varying concentrations of cisplatin, as assessed by flow cytometry. DR5 membrane expression in A2780 cells transfected with siRNA against luciferase (Luc siRNA) or siRNA against DR5 (DR5 siRNA). Cells were pre-incubated with cisplatin for 4 h, washed, cultured in media for 20 h and DR5 membrane expression was determined with flow cytometry. The mean fluorescence intensities (MFI) were corrected for staining with a non-specific isotype control.

B) Apoptosis rate in A2780 DR5 knock-down and control cells in response to TRAIL and cisplatin. A2780 cells transfected with siRNA against luciferase (Luc siRNA) or siRNA against DR5 (DR5 siRNA) were pre-incubated with medium or 30 μM of cisplatin for 4 h and then washed. After 20 h, the cells were exposed for 4 h to 100 ng/ml TRAIL, and apoptosis was assessed by means of acridine orange staining.

FIG. 10. DR5-selective variants are more efficient in DR5-sensitive tumor cell killing than wtTRAIL.

Colo205 cells were treated with increasing concentrations (5-30 ng/ml) of wtTRAIL or DR5-variants and induction of apoptosis was monitored by measuring phosphatidyl serine exposure (A), caspase activation (B) and pro-caspase-8 processing (C). (A) Cell death induction by wtTRAIL and DR5-selective variants, D269H and D269HE195R. The graph shows averaged percentage apoptosis induced±SEM. (B) DEVDase activity in Colo205 cells following treatment with wtTRAIL, D269H and D269HE195R. DEVDase activity was measured in whole-cell lysates with a kinetic assay as described in Example 6. Enzyme activity was expressed in nmole AMC released per min by 1 mg of total cellular protein. (C) Western blot analysis of pro-caspase-8 cleavage in Colo205 cells treated with wtTRAIL and DR5 variants, showing cleavage of caspase-8 at lower concentrations of DR5-specific variants compared to wtTRAIL. The graphs (A, B) show averaged values of three independent experiments, while part C shows one representative picture of two independent experiments.

FIG. 11. Expression of the four TRAIL receptors on the surface of A2780 cells.

Cell surface expression of DR4, DR5, DcR1 and DcR2 was measured by immunostaining followed by flow cytometry as described in Example 6. Each histogram shows an isotype control (black, open peak) and one TRAIL-R labelled (grey, open peak) sample, as indicated on the histograms. The histograms are representatives of three independent experiments.

FIG. 12. DR5-selective variants show lower binding to both DcR1 and DcR2.

Increasing concentration of wtTRAIL, D269H and D269HE195R were used to assess their binding to DcR1 and DcR2 using SPR receptor binding assay. (A) Binding of TRAIL variants to immobilized DcR1-Ig, showing significantly less binding of DR5-selective variants to DcR1 compared to wtTRAIL (B) Binding of TRAIL variants to immobilized DcR2-Ig, showing slightly lower binding of DR5-selective variants to decoy receptor 2 compared to wtTRAIL. The response data (in response units) as a function of TRAIL concentration were fitted using a four parameter equation to give an apparent pre-steady state affinity constant.

FIG. 13. DR5-selective variants are more efficient in A2780 tumor cell killing than wtTRAIL.

(A) Cell death induction by wtTRAIL and D269HE195R. A2780 cells were treated with increasing concentrations (10-50 ng/ml) of wtTRAIL or D269HE195R. Apoptotic cell death was measured 24 h post-treatment by Annexin V assay. (B) DEVDase activity in A2780 cells following treatment with increasing concentrations° of wtTRAIL and D269HE195R for 24 h. DEVDase activity was measured in whole-cell lysates with a kinetic assay as described in Example 6. Enzyme activity was expressed in nmole AMC released per minute by 1 mg of total cellular protein. Graphs A and B show averaged values±SEM from three independent experiments. (C) Western blot analysis of pro-caspase-8 cleavage in A2780 cells treated with increasing concentrations of wtTRAIL and D269HE195R for 24 h, showing cleavage of caspase-8 at lower concentrations of D269HE195R compared to wtTRAIL. The Western blot shows one representative picture of two independent experiments.

FIG. 14. Inhibition of wtTRAIL and DR5-variant-induced apoptosis by decoy receptors.

wtTRAIL, D269H and D269HE195R were pre-incubated for 30 min with increasing concentrations of soluble DcR1- and DcR2-Ig, then added to Colo205 cell cultures at a 10 ng/ml concentration for 3 h. (A) Effect of soluble recombinant DcR1-Ig (sDcR1) on apoptosis induction by TRAIL and DR5-selective variants. The graph shows significantly less cell death in the TRAIL treated group on addition of soluble DcR1 with no effect on D269H or D269HE195R induced cell death measured by Annexin V assay (B) Effect of soluble recombinant DcR2-Ig (sDcR2) on apoptosis induction by TRAIL and DR5-selective variants. The graph shows that higher concentration of sDcR2 is required to reduce apoptosis (measured by Annexin V binding) induced by the DR5-selective variants. (C) Effect of soluble recombinant DR5-Ig (sDR5) on apoptosis induction by TRAIL and DR5-selective variants showing that sDR5 can completely block apoptosis induction by both wtTRAIL and DR5-selective variants. All graphs are representatives of two independent repeats. (D) Neutralizing antibodies against DcR1 and DcR2 can augment wtTRAIL- but not DR5-variant induced cell death. Colo205 cells were incubated for 1 h with increasing concentrations of neutralizing antibodies to DcR1 and DcR2, followed by treatment with wtTRAIL (20 ng/ml) and D269HE195R (4 ng/ml). Induction of apoptosis was measured 3 h post-treatment using Annexin V assay. The graph shows fold increase in apoptosis calculated by dividing the percentage apoptosis induced by wtTRAIL or D269HE195R in the presence of neutralizing decoy receptor antibodies with percentage apoptosis induced by the ligands in the absence of the antibodies. The graphs show the average±SEM of three independent experiments.

FIG. 15. DR5-variants are not toxic to non-transformed cells.

Cells were treated with increasing concentrations of wtTRAIL and DR5-selective variants, D269H and D269HE195R and cell viability was measured using MTT assay, (A) showing no cell death with fibroblast cells and (B) HUVEC's. The graph shows average cell viability±S.D. from three independent experiments as percentage of untreated cells.

FIG. 16. D269HE195R activates death-inducing TRAIL receptors and apoptosis a magnitude faster than wtTRAIL.

Colo205 cells were treated with 30 ng/ml of wtTRAIL or D269HE195R for the times indicated, after which the ligands were washed out and the cells were incubated in normal growth medium for 180 min (for TRAIL receptor activation) or for 240 min (for PS exposure) in total. (A) Kinetics of death-inducing TRAIL receptor activation by wtTRAIL and D269HE195R measured by monitoring pro-caspase-8 processing with Western blotting. (B) Kinetics of cell death induction by wtTRAIL and D269HE195R measured by Annexin V assay. Cell death induction was expressed relative to the percentage of death induced after 240 min without washing out the ligands. The graph shows the average of two independent experiments±SEM (C) Binding competition between wtTRAIL and D269HE195R to cell surface-expressed receptors. wtTRAIL (30 ng/ml) was added to Colo205 cell culture for 5 or 10 min. Unbound wtTRAIL washed out and 30 ng/ml D269HE195R was added to the cells for another 5 or 10 min, and then removed.

The cells were incubated for 180 min in total in normal growth medium, when induction of cell death was determined by Annexin V assay. The graph shows the average percentage of apoptosis induced±SEM from three independent experiments.

FIG. 17. Receptor expression in Colo205 cell line following treatment with Aspirin and 5-Flurouracil

Colo205 cell line was treated with 5 mM Aspirin for 24 h or 10 μM of 5-Flurouracil for 24 h, and then harvested for receptor expression for TRAIL R1, R2, R3 and R4. Aspirin leads to induction TRAIL R2, R3 and R4 receptor expression. 5-Flurouracil leads to an induction of all TRAIL receptors.

FIG. 18. D269HE195R used in combination with aspirin result in enhanced synergism compared to WT TRAIL.

(A) Aspirin induces expression of DR5, DcR1 and DcR2 on the cell surface. Colo205 cells were treated with 5 mM aspirin for 24 h and cell surface expression of DR4, DR5, DcR1 and DcR2 was measured by immunostaining and detected with flow cytometry. The histograms shown include isotype control (black, open peak), untreated Colo205 (grey, closed peak) and aspirin-treated Colo205 (grey, open peak) samples. The histograms are representatives of three independent experiments. (B) Caspase activation by combined treatment with aspirin plus wtTRAIL or D2691-1E195R. Cells were treated with 5 mM aspirin for 24 h followed by 5 or 10 ng/ml of wtTRAIL or D269HE195R for the times indicated, after which the cells were harvested and DEVDase activity was measured in the cell lysates. DEVDase activity was expressed as average nmol AMC released per minute by 1 mg total cellular protein±SD of 3 independent experiments.

FIG. 19. Comparison of 2 and 5×10⁶ Colo205 cells for inoculation.

Athymic nude mice were subcutaneously injected with 200 μl of medium containing 2 or 5*10̂6 of Colo205 cells and tumour growth was followed for several weeks.

FIG. 20. Aspirin as single treatment on Colo205 tumors.

Two concentrations of aspirin, 40 and 200 mg/kg, were tested in a small number of athymic nude mice. Aspirin was IP injected daily for a 2×5 days period with 2 days in between.

FIG. 21. In vivo effect of TRAIL on Colo205 tumors.

The anti-tumor effect of rhTRAIL WT and D269HE195R was tested in a small number of athymic nude mice carrying a tumor of Colo205 cells (n=3 to 5 per group). The rhTRAIL proteins were tested in doses of 1 and 5 mg/kg, given by intraperitoneal injections for 2×5 days (day 1-5 and 8-12) with a daily doses of aspirin (in the same syringe as protein).

FIG. 22. Cell survival assays looking at the effect of TRAIL/DR5 mutants on viability of Colo205 cells with and without combination therapy either with Aspirin or 5-Flurouracil

(A) DR5 mutants (D269H, D269HE195R and M1) lead to, significantly less cell viability than TRAIL with the largest differences seen at lower concentrations of the ligand. (B) Effect of increasing concentrations of Aspirin on cell viability. (C) Effect of increasing concentrations of 5-Flurouracil on cell viability. (D) Combination therapy with 5 mM Aspirin pre-incubated for 24 h lead to further decreases in cell viability in cells treated with both TRAIL and two of the DR5 mutants. (E) 30 minute incubation of DR5 mutants, were sufficient decrease cell viability which was further augmented when cells were pre-incubated for 24 h with Aspirin. The shorter incubation period lead to a decrease potency of TRAIL alone or in combination with Aspirin. (F) Combination therapy with 10 μM 5-Flurouracil pre-incubated for 24 h did not lead to any significant enhancement in both TRAIL and DR5 mutant treated cells. (G) 30 min incubation of the DR5 mutant was sufficient to decrease cell viability which was not enhanced by 5-Flurouracil, and once again the decease potency of TRAIL is seen. Proteins are indicated as follows: TRAIL stands for rhTRAIL WT; D269H stands for rhTRAIL carrying mutation D269H; E195R stands for rhTRAIL carrying mutations D269H and E195R; and M1 stands for rhTRAIL carrying mutations D269H, E194I and I196S.

FIG. 23. Effect of wtTRAIL, single therapy and combination with aspirin on Colo205 tumors.

The anti-tumor effect of rhTRAIL WT was tested in a small number of athymic nude mice carrying a tumour of Colo205 cells (n=3 to 5 per group) as a single therapy and in combination with 200 mg/kg aspirin. The rhTRAIL protein was tested in doses of 1 and 5 mg/kg, given by intraperitoneal injections for 2×5 days (day 1-5 and 8-12) with or without the doses of aspirin (in the same syringe as protein). ASA indicates combination with aspirin.

FIG. 24. Effect of D269HE195R TRAIL, single therapy and combination with aspirin on Colo205 tumors.

The anti-tumor effect of D269HE195R was tested in a small number of athymic nude mice carrying a tumour of Colo205 cells (n=3 to 5 per group) as a single therapy and in combination with 200 mg/kg aspirin. The rhTRAIL protein were tested in doses of 1 and 5 mg/kg, given by intraperitoneal injections for 2×5 days (day 1-5 and 8-12) with or without the doses of aspirin (in the same syringe as protein). ASA indicates combination with aspirin.

FIG. 25. Apoptosis assays showing enhanced effect of TRAIL and DR5 mutants with Aspirin, with the mutants showing more apoptosis compared to TRAIL.

Apoptosis was measured using AnnexinV/PI staining at 3 h following the addition of 5 and 10 ng/ml of TRAIL or the DR5 mutant (D269HE195R), in cells which were pre-incubated with or without 5 Mm Aspirin or 10 μM 5-Flurouracil. The DR5 mutant treated cells display more apoptosis which can be enhanced by Aspirin and not 5-Flurouracil. TRAIL treated cells show lesser apoptosis which can be enhanced by Aspirin and to a lesser extent by 5-Flurouracil.

FIG. 26. DR5 mutants show greater caspase activation compared to TRAIL which can be enhanced with Aspirin

Colo205 cells were harvested at various time points shows a greater caspase activation following treatment with 10 ng/ml of the DR5 mutant (D269HE195R). .The caspase activity is further enhanced in cells pre-incubated for 24 h with 5 mM of Aspirin but not 5-Flurouracil. Minimal caspase activation is seen for the given time points in cells treated with 10 ng/ml of TRAIL.

FIG. 27. Receptor expression in SW948 cell line following treatment with Aspirin and 5-Flurouracil

SW948 cell line was treated with 2.5 mM Aspirin for 24 h or 10 μM of 5-Flurouracil for 24 h, and then harvested for receptor expression for TRAIL R1, R2, R3 and R4. Aspirin leads to induction TRAIL-R2 expression only. 5-Flurouracil did not lead to any induction of the TRAIL receptors.

FIG. 28. Cell survival assays looking at the effect of TRAIL/DR5 mutants on viability of SW948 cells with and without combination therapy either with Aspirin or 5-Flurouracil

(A) TRAIL and the DR5 mutants (D269H, D269HE195R and MD lead to decreased cell viability with TRAIL leading to greater loss in cell viability compared to that of the DR5 mutant at higher concentrations. (B) Effect of increasing concentration of Aspirin on SW948 cell. (C) Combination therapy with 2.5 nM Aspirin pre-incubated for 24 h lead to further decreases in cell viability in cells treated with both TRAIL and two of the DR5 mutants. (D) Combination therapy with 10 μM 5-Flurouracil pre-incubated for 24 h lead to significant enhancement only in TRAIL treated cells.

FIG. 29. Apoptosis assays showing enhanced effect of the TRAIL and DR5 mutants with Aspirin, with 5-Flurouracil enhancing only TRAIL treated cells.

Apoptosis was measured using AnnexinV/PI staining at 3 h following the addition of 5 and 10 ng/ml of TRAIL or the. DR5 mutant (D269HE195R), in cells which were pre-incubated with or without 5 Mm Aspirin or 10 μM 5-Flurouracil. The DR5 mutant treated cells display more apoptosis which can be enhanced by Aspirin and not 5-Flurourail. TRAIL treated cells show lesser apoptosis which can be enhanced by Aspirin and to a lesser extent by 5-Flurouracil.

FIG. 30. TRAIL treated cells showed enhanced caspase activity when combined with 5-Flurouracil

Caspase activity measured at 3 h showing following treatment with 20 ng/ml TRAIL or DR5 mutant (D269HE195R). Cells pre-incubated with 10 μM 5-Flurouracil showed enhanced caspase activity following treatment with TRAIL but not the DR5 mutant. Pre-incubation with 2.5 mM Aspirin showed no further enhanced caspase activity compared to either ligand alone at this time point.

FIG. 31. Receptor expression in LOVO cell line following treatment with Aspirin and 5-Flurouracil

Lovo cell line was treated with 2.5 mM Aspirin for 24 h or 10 μM of 5-Flurouracil for 24 h, and then harvested for receptor expression for TRAIL R1, R2, R3 and R4. Aspirin leads to an induction TRAIL R4 and to a lesser extent TRAIL R2 receptor expression. 5-Flurouracil leads to an induction of TRAIL R2, R3 and R4 receptors.

FIG. 32. Cell survival assays looking at the effect of TRAIL/DR5 mutants on viability of Lovo cells with and without combination therapy either with Aspirin or 5-Flurouracil

(A) TRAIL and the DR5 mutants (D269H, D269HE195R and M1) lead to an equal decrease in cell viability (B) Effect of increasing concentration of Aspirin on Lovo cell. C) Effect of increasing concentration of 5-Flurouracil on Lovo cells. (D) Pre-incubation with 2.5 mM Aspirin for 24 h lead to significantly decreased cell survival in both TRAIL and DR5 mutant treated cells. (E) Pre-incubation with 10 μM 5-Flurouracil for 24 h lead to significantly decreased cell survival in both TRAIL and DRS mutant treated cells.

FIG. 33. Apoptosis assays showing enhanced effect of the TRAIL and DR5 mutants with Aspirin and 5-Flurouracil

Apoptosis was measured using AnnexinV/PI staining at (A) 7 and (B) 15 h following the addition of 5 and 50 ng/ml of TRAIL or the DR5 mutant (D269HE195R), in cells which were pre-incubated with or without 5 Mm Aspirin or 10 μM 5-Flurouracil. Enhanced apoptosis in cells pretreated with Aspirin and 5-Flurouracil following TRAIL and DR5 mutant (D269HE195R) treatment. Enhanced apoptosis with Aspirin is best seen at 15 h.

FIG. 34. Earlier and enhanced caspase activity is seen in DR5 mutant treated Lovo cells pre-incubated with aspirin and 5-Flurouracil

Lovo cells were harvested at various time points, shows an earlier caspase activation following treatment with 50 ng/ml of DR5 mutant (D269HE195R) compared to TRAIL 50 ng/ml. The caspase activity is further enhanced in cells pre-incubated for 24 h with 5 mM of Aspirin and 10 μM 5-Flurouracil.

FIG. 35. Cell survival assays looking at the effect of TRAIL/DR5 mutants on viability of HCT15, HT29 and RKO cells with and without combination therapy either with 5-Flurouracil

TRAIL and DR5 mutant (D269HE195R) show equal decrease in cell viability following treatment with or without combination therapy with 5-Flurouracil. 5-Flurouracil does not further decrease cell viability compared to ligand alone in all cell lines tested (A) & (B) HCT15, (C) RKO and (D)HT29.

FIG. 36. Top (left) and side (right) view of the trimer-trimer complex of the extracellular moiety of wtTRAIL (grey) and of DR5 (black).

The side chains of amino acids E195 and D269 are indicated by spheres. Mutation of these amino acids to arginine and histidine, respectively, convert wtTRAIL from a promiscuous cytokine into a DR5-specific variant¹⁰.

Table 4—Sensitivity of colon carcinoma cell lines to wtTRAIL & DR5-selective TRAIL variant

FIG. 37. SPR analysis of the binding of wtTRAIL and D269HE195R to immobilized receptor.

A) typical sensorgrams of the binding of wtTRAIL to directly immobilized DR4-Ig. B) Pre-steady state analysis of the binding of wtTRAIL and D269HE195R to DR4-Ig (left panel) and DR5-Ig (right panel) directly immobilized to the surface of a CM5 chip. All binding responses were plotted relative to the value of wtTRAIL at 250 nM (100%).

FIG. 38. Dose-response curve of the apoptotic activity of wtTRAIL and D269HE195R in colon carcinoma cell line Colo205.

TRAIL-induced cell death as a function of concentration was measured with the MTS assay.

FIG. 39. Induction of p53 following treatment with Bortezomib in cervical cancer cell lines.

All three cervical cancer cell lines were treated with increasing concentration of Bortezomib to assess expression of p53 protein. (A) Induction of p53 protein can be seen at concentrations of 1 nM with largest induction seen at 5 and 10 nM in Caski cell line (B) Induction of p53 in SIHA cells can be seen at 5 and 10 nM. (C) In HELA cells induction of p53 is seen also at 5 and 10 nM, though relatively smaller to that of SIHA and Caski cell lines.

FIG. 40. Receptor expression in Caski cell line following treatment with Bortezomib and Radiotherapy

Caski cervical cell line was treated with (A) 5 nM Bortezomib for 24 h or (B) incubated for 24 h following 10Gy radiotherapy, and then harvested for receptor expression for TRAIL R1, R2, R3 and R4. (A) Bortezomib treatment increased receptor expression of TRAIL R2 and slight increase of TRAIL R3. (B) Radiotherapy only leads to slight increase TRAIL R3 receptor expression, with no change in other receptor expressions.

FIG. 41. Cell survival assays looking at the effect of TRAIL/DR5 mutants on viability of Caski cells with and without combination therapy either with Bortezomib of Radiotherapy

(A) TRAIL and DR5 mutants (D269H, D269HE195R and MD appear comparable as a single agent in affecting cell viability. (B) Effect of increasing concentrations of Bortezomib on cell viability. (C) Combination therapy with 5 nM Bortezomib incubated for 24 h lead to further decreases in cell viability in cells treated with both wtTRAIL and two of the DRS mutants (D) Radiation had no enhancing effect in combination with wtTRAIL or the DR5 mutants.

FIG. 42. Apoptosis assays showing enhanced effect of TRAIL and DR5 mutant with Bortezomib

(A) AnnexinV/PI staining and (B) measured at 7 h following TRAIL and DR5 mutant (D269HE195R) treatment in Caski cells preincubated with or without Bortezomib for 24 h. Both ligands appears to show enhanced effects with Bortezomib, with the DR5 mutant showing more apoptosis compared to TRAIL at the give time point. Radiotherapy had no enhancing effect compared to cells treated with TRAIL or DR5 mutant alone (data not shown).

FIG. 43. Both TRAIL and DR5 mutant show enhance caspase activity to Bortezomib, with DR5 mutant showing earlier caspase activation

Caski cells harvested at various time points to show enhanced caspase activity with Bortezomib in both TRAIL and DR5 mutant treated cells. Concentration of ligand used was 50 ng/ml. The DR5 mutants however show an earlier caspase activation compared to the TRAIL treated samples.

FIG. 44. Receptor expression in SIHA cell line following treatment with Bortezomib and Radiotherapy

SIHA cervical cell line was treated with (A) 10 nM Bortezomib for 24 h or (B) incubated for 24 h following 10Gy radiotherapy, and then harvested for receptor expression for TRAIL R1, R2, R3 and R4. (A) Bortezomib treatment increases receptor expression of all TRAIL receptors. (B) Radiotherapy only leads to an increase TRAIL R2 and TRAIL R3 receptor expression, with no change in other receptor expressions.

FIG. 45. Cell survival assays looking at the effect of TRAIL/DR5 mutants on viability of SIHA cells with and without combination therapy either with Bortezomib of Radiotherapy

(A) Effect of increasing concentration of Bortezomib on cell survival. (B) SIHA cells are resistant to TRAIL and DR5 mutant treatment, however, when incubated for 24 h with 5 nM of Bortezomib, we demonstrate decreased in cell survival following treatment of both TRAIL and the DR5 mutants. (C) 10Gy Radiotherapy had no effect in further decreasing cell viability of SIHA cells in both TRAIL and DR5 mutant treated cells.

FIG. 46. DR5 mutant shows a greater apoptosis compared to TRAIL in SIHA cells following incubation with Bortezomib.

Apoptosis was measured using Acridine Orange staining and cells were measured 7 h following treatment with TRAIL and DR5 mutant D269HE195R, in SIHA cells with or without pre-incubation for 24 h with 10 nM Bortezomib. The DR5 mutant show greater apoptosis compared to TRAIL at the given time point. Radiotherapy had no enhancing effect compared to cells treated with TRAIL or DR5 mutant alone (data not shown).

FIG. 47. Both TRAIL and DR5 mutant show enhance caspase activity to Bortezomib, with DR5 mutant showing earlier caspase activation

SIHA cells harvested at various time points to show enhanced caspase activity with Bortezomib in both TRAIL and DR5 mutant treated cells. Concentration of ligand used was 50 ng/ml. The DR5 mutants however show an earlier and greater caspase activation compared to the TRAIL treated samples.

FIG. 48. Receptor expression in HELA cell line following treatment with Bortezomib and Radiotherapy

Hela cervical cell line was treated with (A)10 nM Bortezomib for 24 h or (B) incubated for 24 h following 10Gy radiotherapy, and then harvested for receptor expression for TRAIL R1, R2, R3 and R4. (A) Bortezomib treatment increases receptor expression of all TRAIL receptors. (B) Radiotherapy also leads to an increase of all TRAIL receptors.

FIG. 49. Cell survival assays looking at the effect of TRAIL/DR5 mutants on viability of HELA cells with and without combination therapy with either Bortezomib of Radiotherapy

(A) TRAIL and DR5 mutants (D269H, D269HE195R and M1) appear comparable as a single agent in affecting cell viability. (B) Effect of increasing of Bortezomib on cell viability. Hela cells appear to tolerate higher concentration of Bortezomib compared to Caski and SIHA cells (C) Combination therapy with 10 nM Bortezomib incubated for 24 h lead to further decreases in cell viability in cells treated with both TRAIL and two of the DR5 mutants (D) Radiation therapy in combination with TRAIL or the DR5 mutants further decrease cell viability in HELA cells. The DR5 mutant show decrease cell viability at lower concentration of ligand compared to TRAIL.

FIG. 50. Apoptosis assay showing enhanced effect of TRAIL and DR5 mutant with Bortezomib and 10Gy Radiotherapy

Apoptosis was measured using Acridine Orange staining and cells were measured 7 h following treatment with TRAIL and DR5 mutant D269HE195R, in Hela cells with or without pre-incubation for 24 h with 10 nM Bortezomib or 10Gy Radiotherapy. Both TRAIL and the DR5 mutant show almost equal enhancement of apoptosis with either combination therapy. Combination of Bortezomib, Radiotherapy and TRAIL or DR5 mutant had no further additive effect.

FIG. 51. Both TRAIL and DR5 mutant show enhance caspase activity to Bortezomib and Radiotherapy.

Hela cells harvested at various time points to show enhanced caspase activity with Bortezomib in both TRAIL and DR5 mutant treated cells. Concentration of ligand used was 50 ng/ml. TRAIL appears to show and earlier and greater caspase activation compared to the DR5 mutant when combined with radiotherapy.

FIG. 52. Human colon adenoma cells are sensitive to TRAIL and D269H/E195R.

The colon adenoma cell lines VACO-235 and VACO330 were treated with TRAIL or D269H/E195R for 96 hours. The adenoma cell lines were more sensitive to D269H/E195R. Sensitivity could be further enhanced using TRAIL or D269H/E195R in combination with the cdk inhibitor roscovitine.

Table 5—Sensitivity of cervical carcinoma cell lines to wtTRAIL & DR5-selective TRAIL variant.

Table 6—Sensitivity of ovarian carcinoma cell line A2780 to wtTRAIL & DR5-selective TRAIL variant

FIG. 53. FIG. 53: P-Akt inhibition for 17 h selectively sensitizes Colo205 colon carcinoma to TRAIL-DR5 (D269H/E195R)

Modulation of TRAIL and D269H/E195R induced apoptosis by PI3K inhibition in Colo205, as assessed by Annexin V assay. Cells were pre-incubated for 15-18 hours with 20 μM LY294002, then either left untreated or exposed to rhTRAIL (0.1 μg/ml) or D269H/E195R (0.1 μg/ml) for 3 additional hours before harvest. While sensitivity to TRAIL slightly decreased, sensitivity to D269H/E195R was enhanced.

FIG. 54. HeLa xenografts treated with wtTRAIL or TRAIL-DR5 (D269H/E195R).

Xenografts of the HeLa cervical cancer cell line were established with matrigel in athymic nude mice. Treatment was started when tumor volume was +/−150 mm3. No effect was observed in HeLa cervical cancer xenografts following treatment of TRAIL or (D269H/E195R).

FIG. 55. Mathematical modeling of TRAIL receptor complex formation triggered by wtTRAIL and D269HE195R over time.

(a) Formation of ligated, homotrimeric DR4, DR5, DcR1 and DcR2 complexes after exposure to wtTRAIL or (b) D269H/E195R. (c) Formation of ligated, homotrimeric DR4 and DR5, in the absence of decoy receptors after exposure to wtTRAIL or (d) D269HE195R.

FIG. 56. Mathematical simulation of ligand-induced TRAIL receptor complex formation over time. (a) Homotrimeric DR4, DRS, DcR1 and DcR2 formation by a theoretical TRAIL variant, which binds to DR5 with the affinity of D269HE195R and with the affinity of wtTRAIL to the other three TRAIL receptors. (b, c) Effect of ligand-removal after 300 sec incubation on the formation of homotrimeric receptor complexes using wtTRAIL (b) or D269HE195R (c). (d, e) Formation and disassembly of heteromeric receptor trimers during incubation with (d) wtTRAIL or (e) D269HE195R.

Table 7. Kinetic constants used for the TRAIL receptor binding simulation.

[symbol]

### EXAMPLES

Example 1

### Expression And Purification of Wild-Type TRAIL And Mutants

The wild-type TRAIL and TRAIL mutant constructs were transformed to Escherichia Coli BL21 (DE3) (Invitrogen). Wild-type TRAIL and M1 were grown at a 5 l batch scale in a 7.5 l fermentor (Applicon) using 4×LB medium, 1% (w/v) glucose, 100 μg/ml ampicillin and additional trace elements. The culture was grown to mid-log phase at 37° C., 30% oxygen saturation and subsequently induced with 1 mM IPTG. ZnSO₄ was added at a concentration of 100 μM to promote trimer formation. Temperature was lowered to 28° C. and the culture was grown until stationary phase. Other mutants were grown in shake flasks at a 1 l scale at 250 rpm, using a similar protocol. Protein expression was induced when the culture reached OD₆₀₀ 0.5 and induction was continued for 5 h. In this case, the medium used was 2×LB without additional trace elements.

The isolated pellet was resuspended in 3 volumes extraction buffer (PBS pH 8, 10% (v/v) glycerol, 7 mM β-mercapto-ethanol). Cells were disrupted using sonication and extracts were clarified by centrifugation at 40,000 g. Subsequently, the supernatant was loaded on a nickel charged IMAC Sepharose fast-flow column and wild-type TRAIL and TRAIL mutants were purified as described by Hymowitz²⁴ with the following modifications: 10% (v/v) glycerol and a minimal concentration of 100 mM NaCl were used in all buffers. This prevented aggregation during purification. After the IMAC fractionation step, 20 μM ZnSO₄ and 5 mM of DTT (instead of β-mercapto-ethanol) was added in all buffers. Finally, a gelfiltration step, using a Hiload Superdex 75 column, was included. Purified proteins were more than 98% pure as determined using a colloidal coomasie brilliant blue stained SDS-PAGE gel. Purified protein solutions were flash frozen in liquid nitrogen and stored at −80° C.

Example 2

### In vitro activity of TRAIL-DR5, TRAIL and cisplatin on A2780

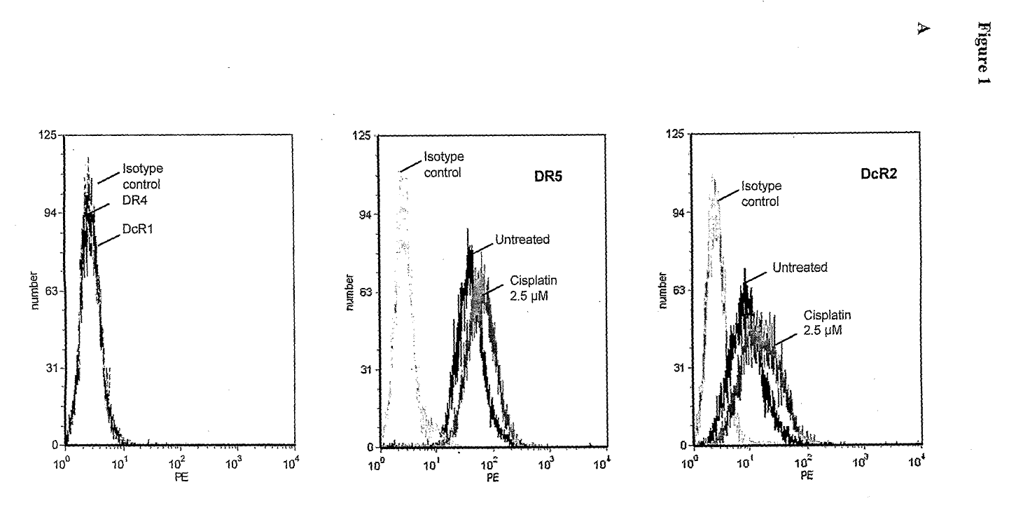

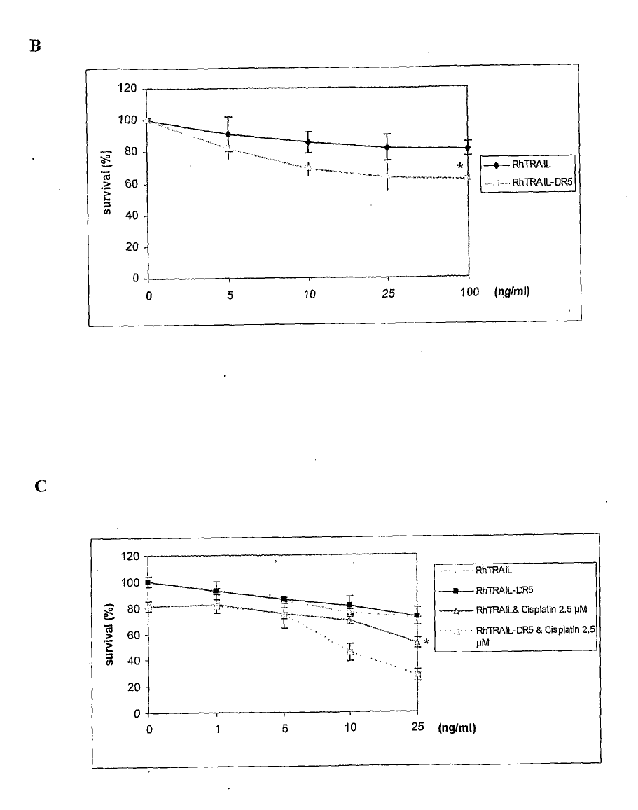

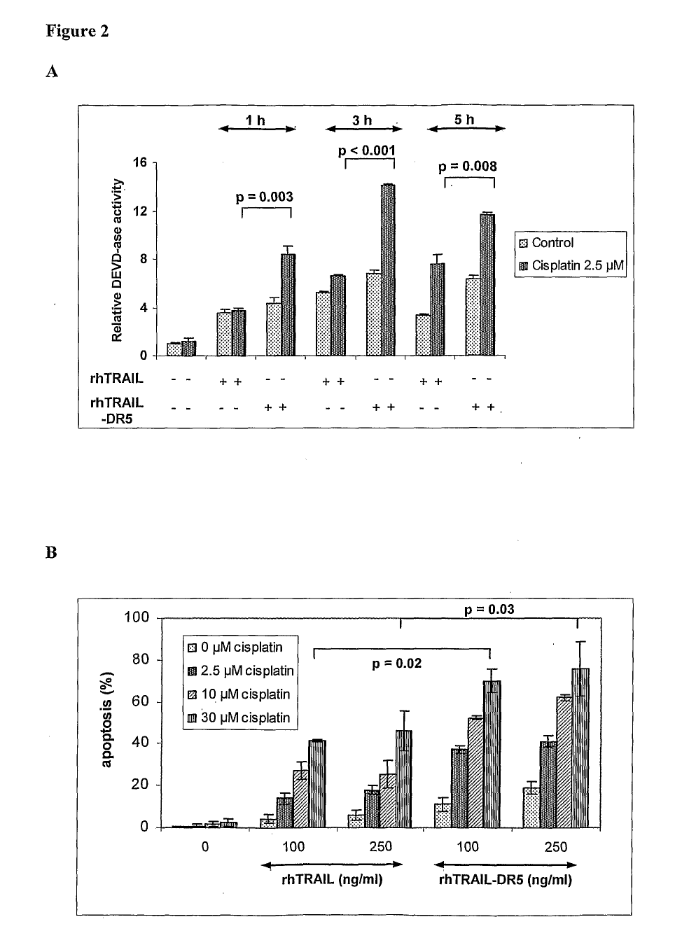

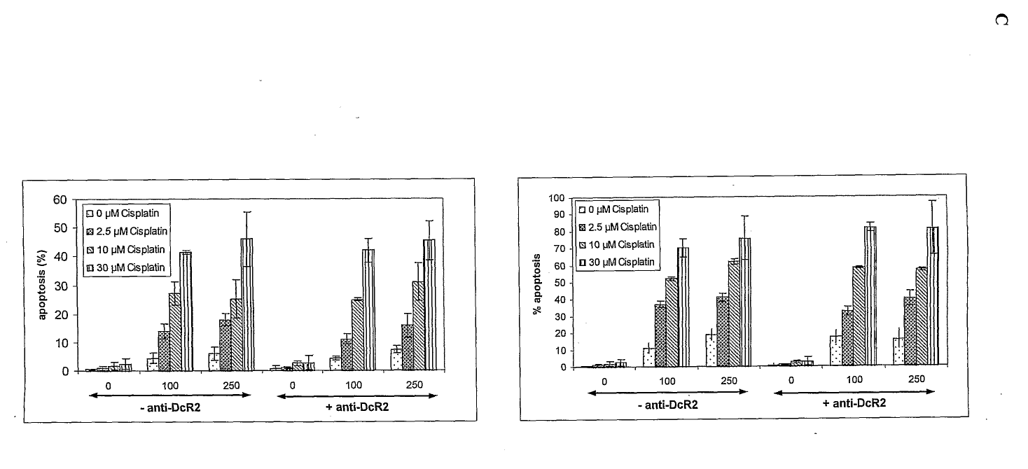

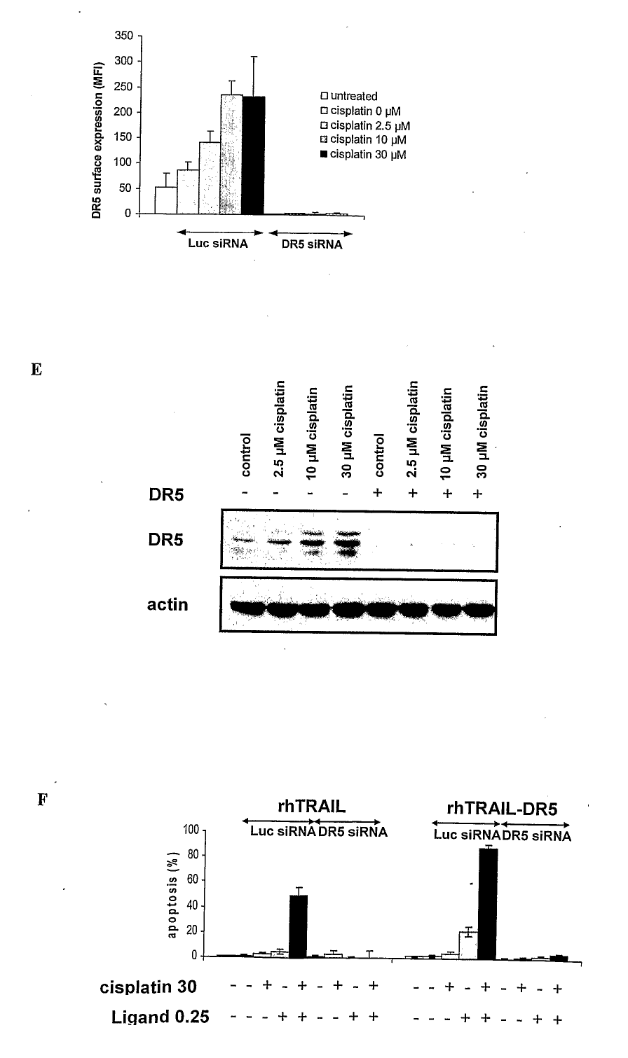

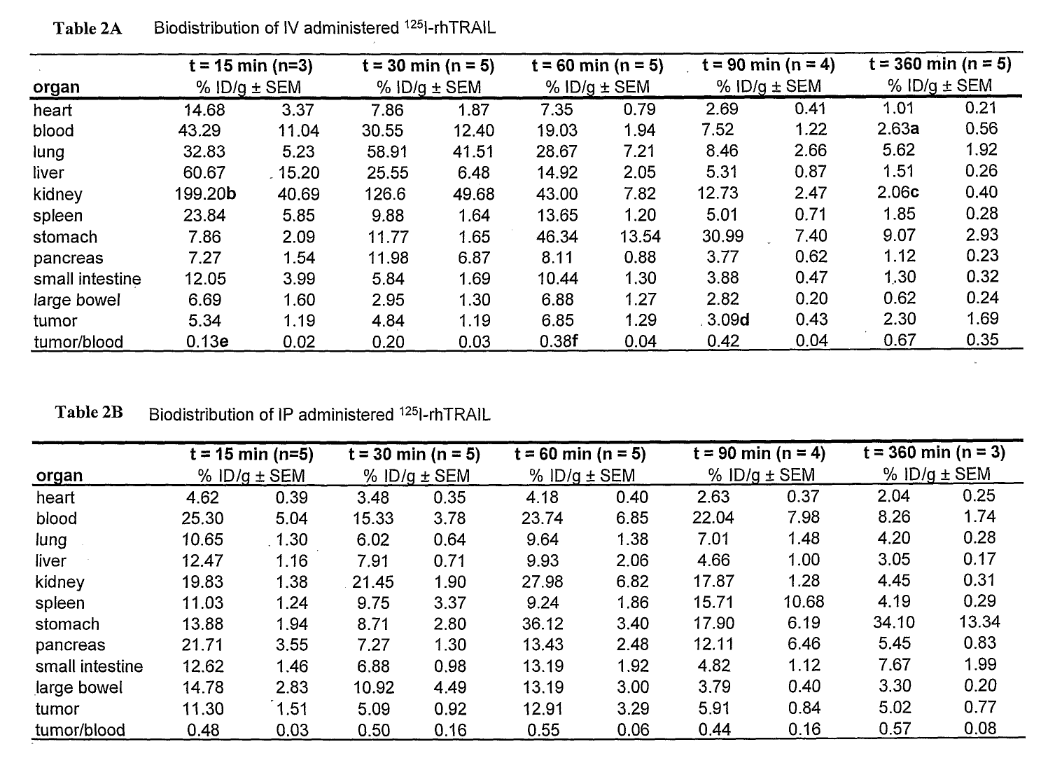

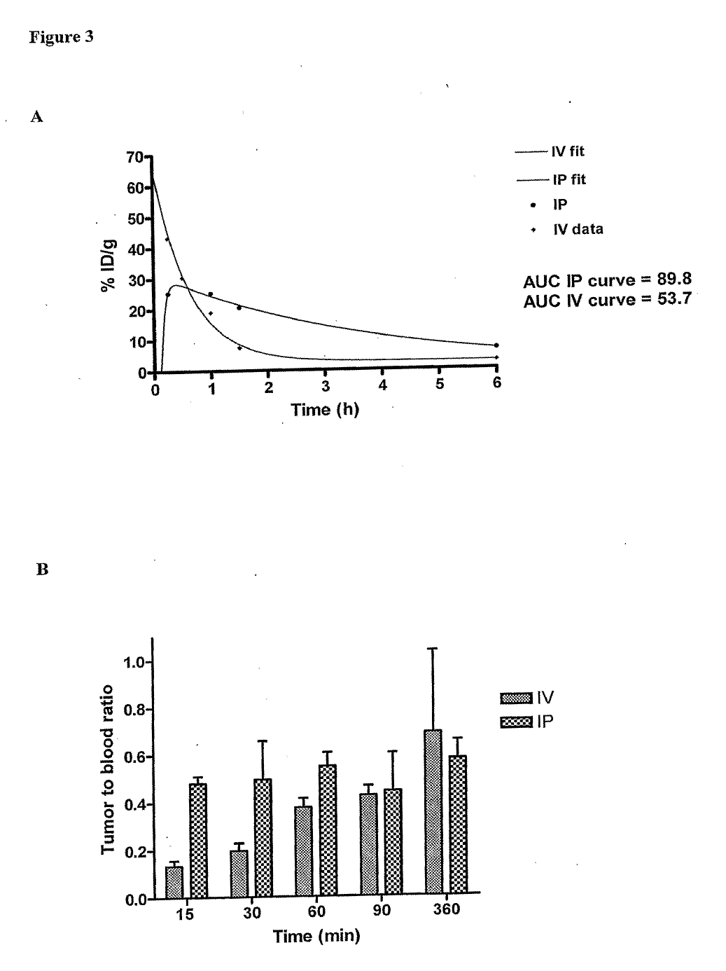

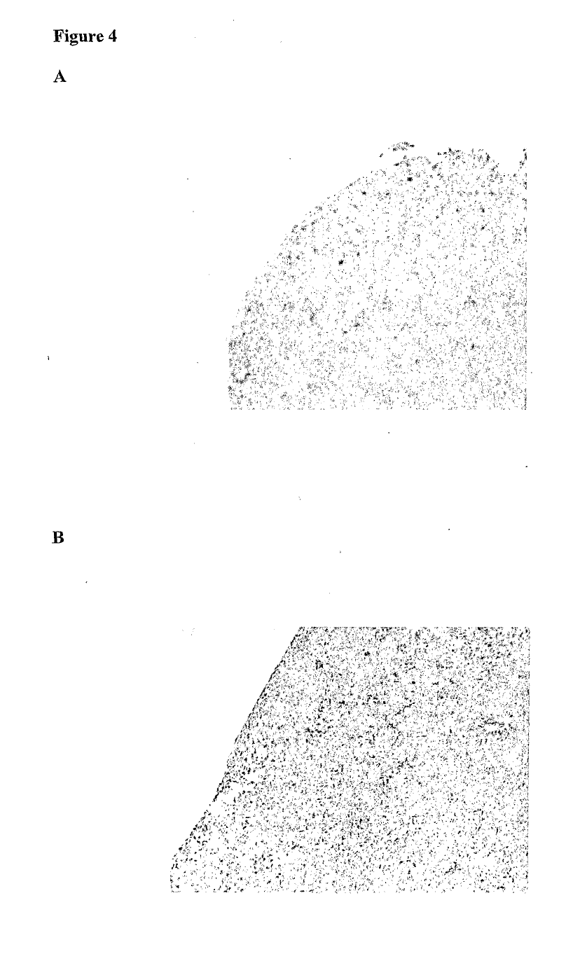

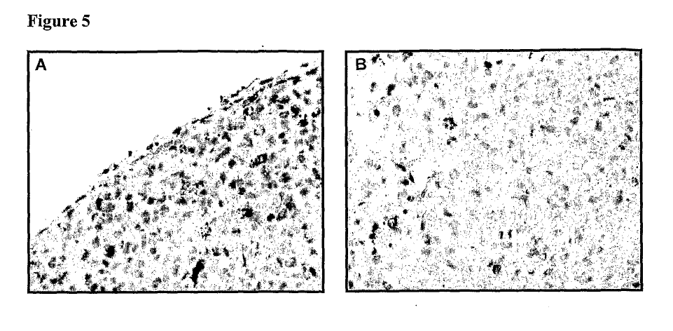

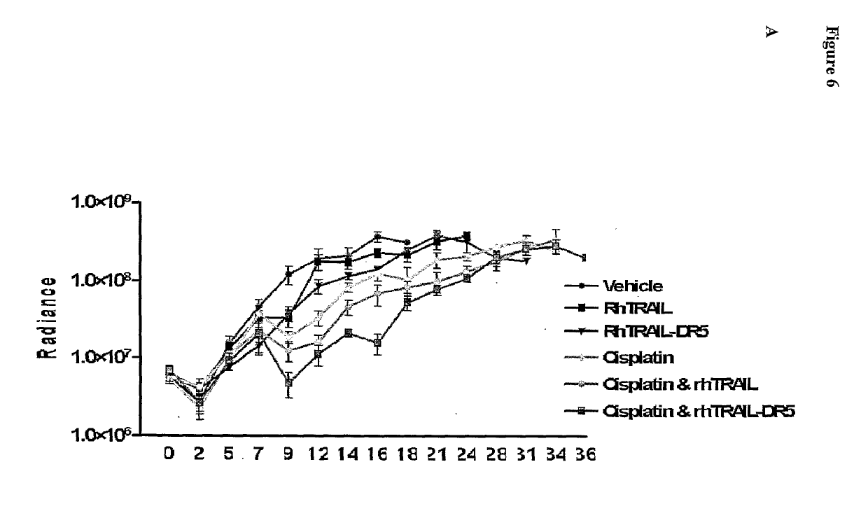

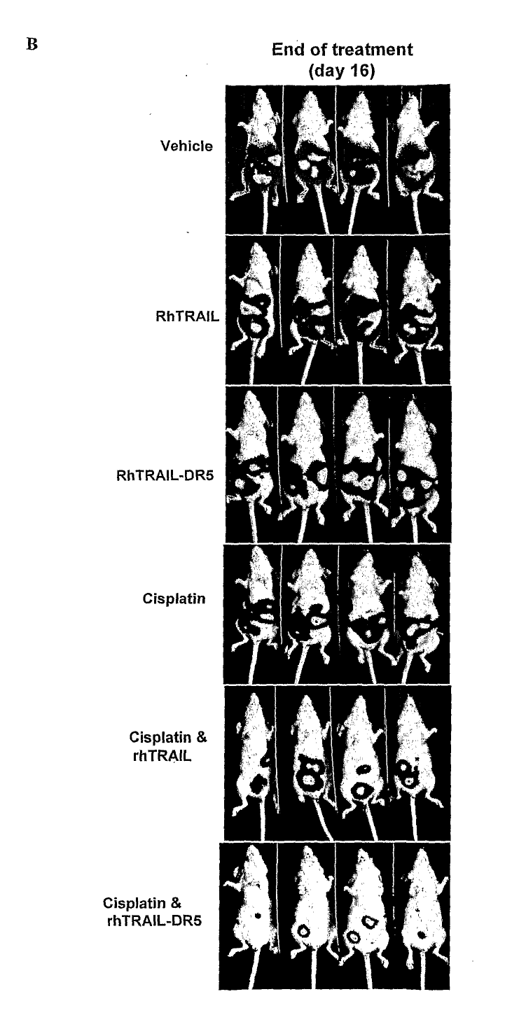

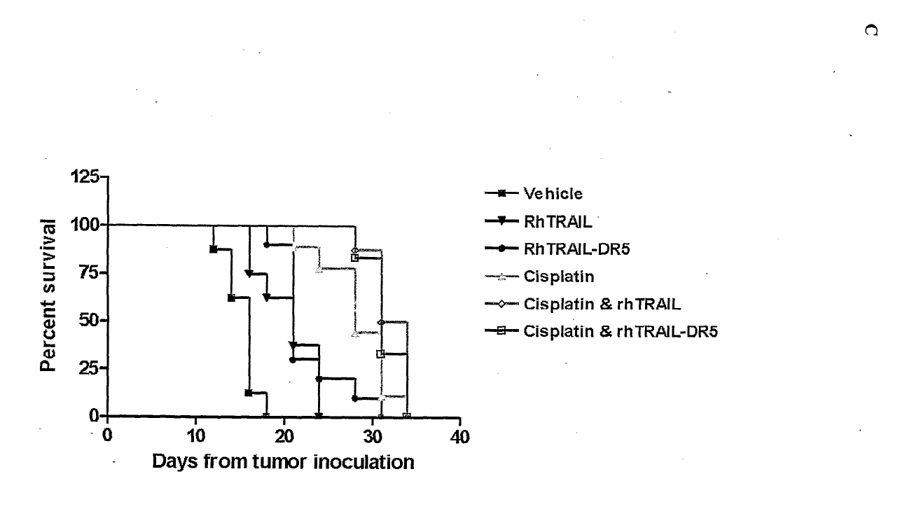

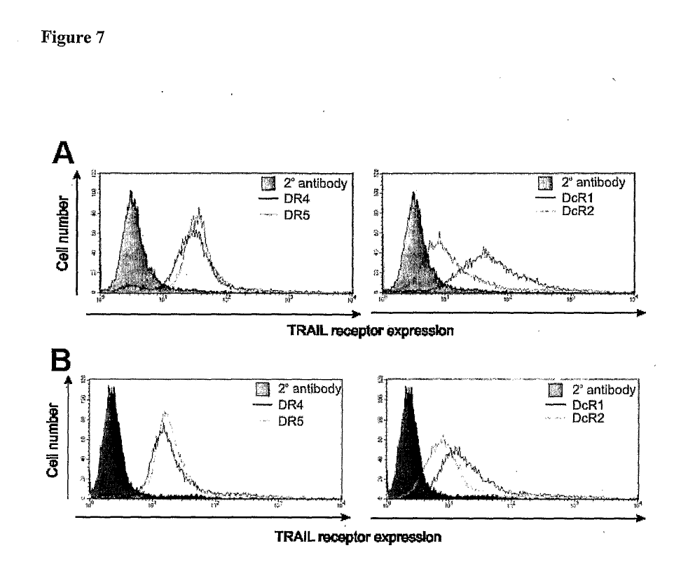

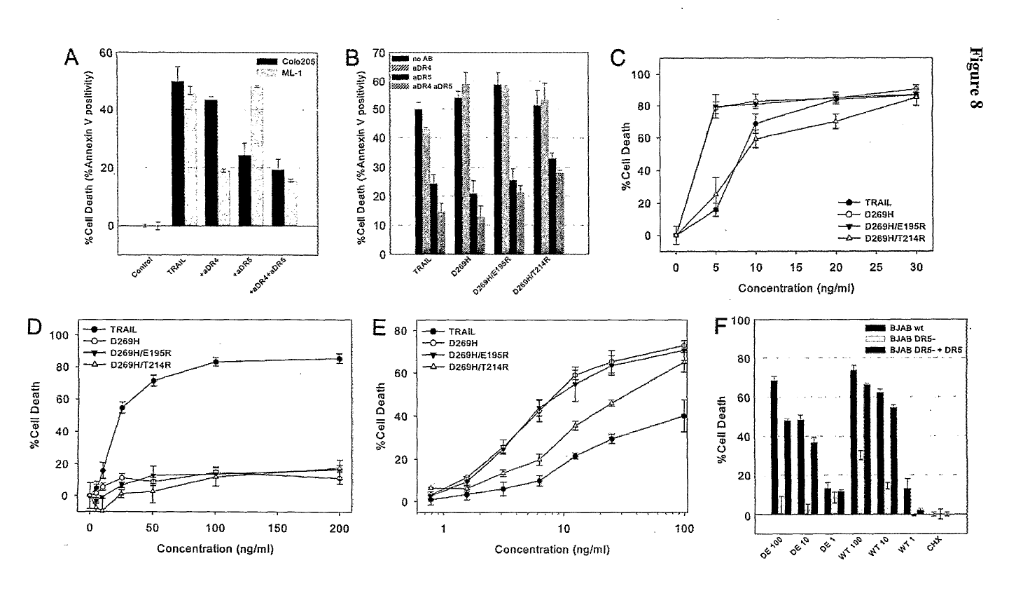

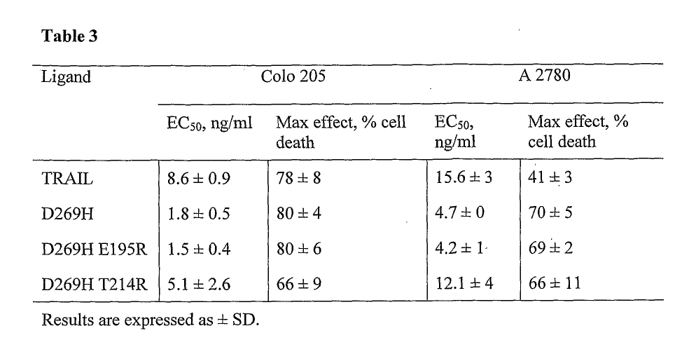

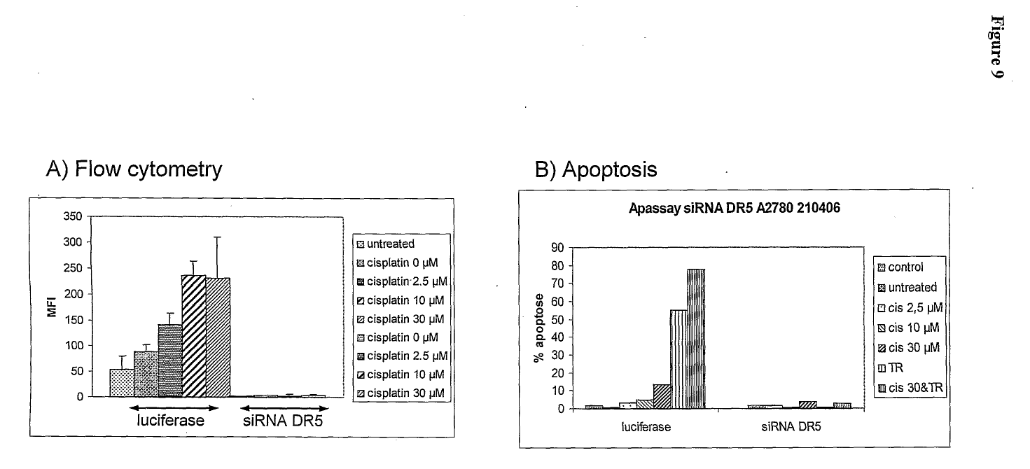

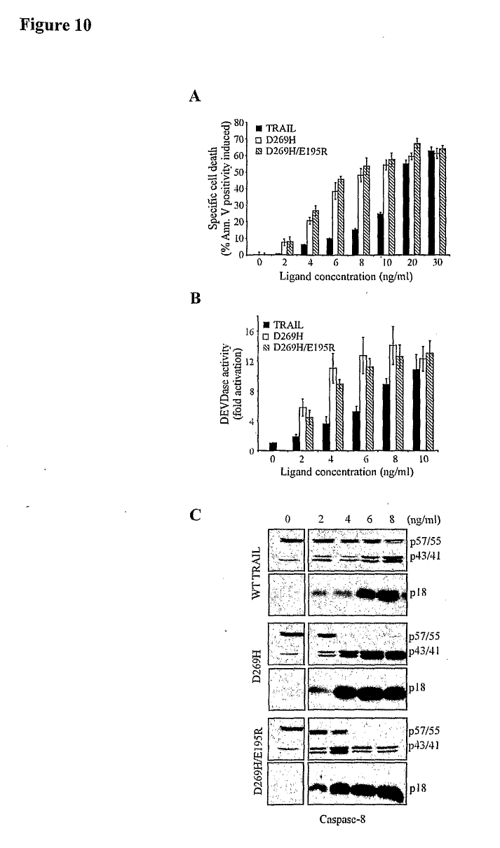

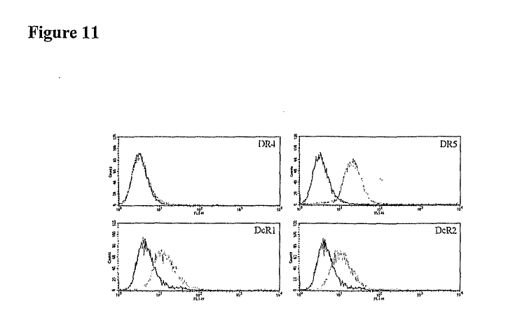

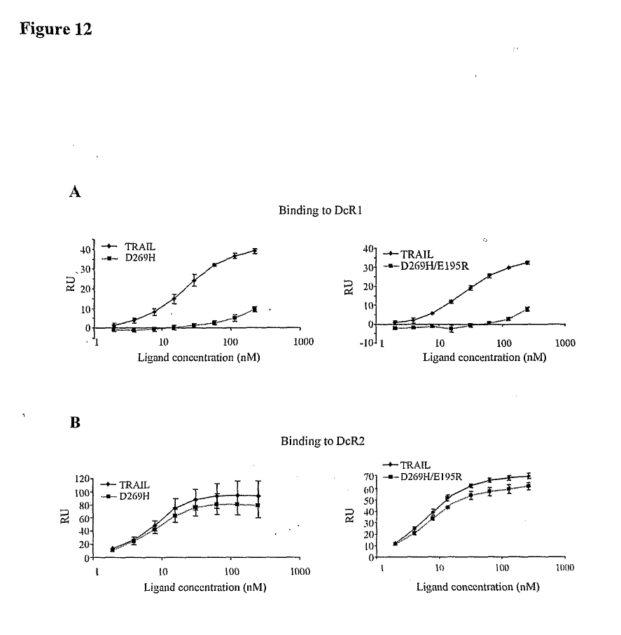

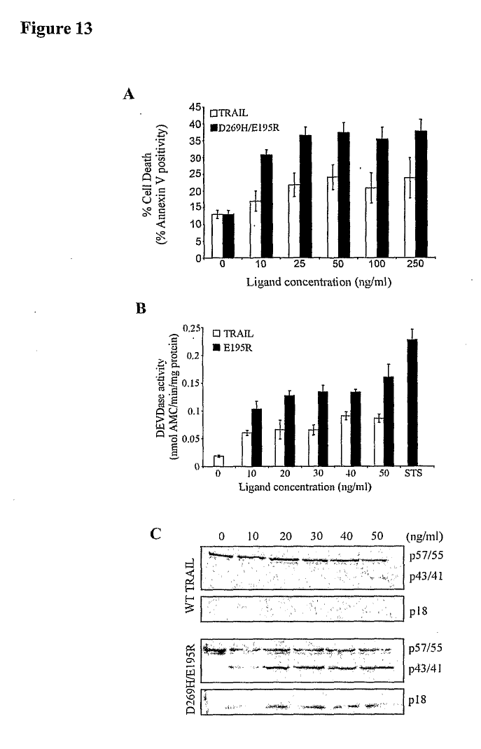

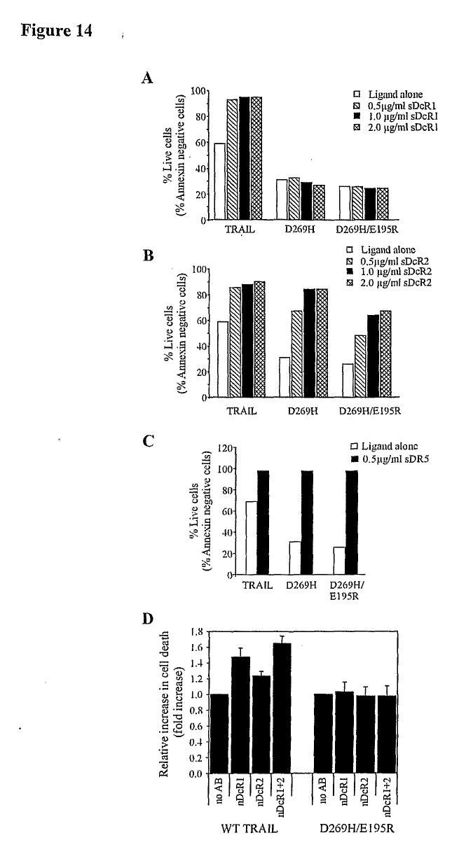

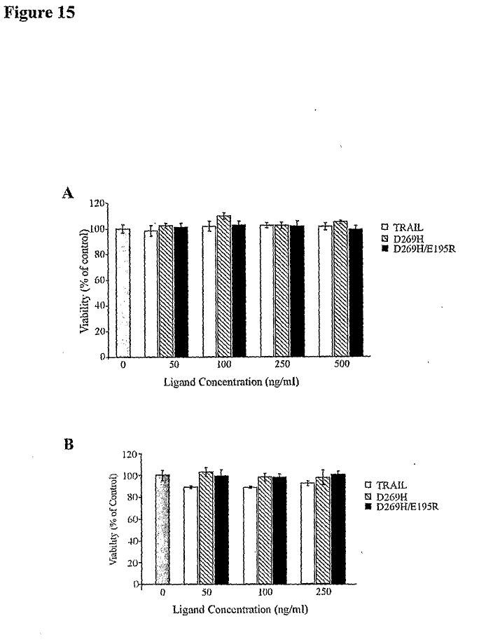

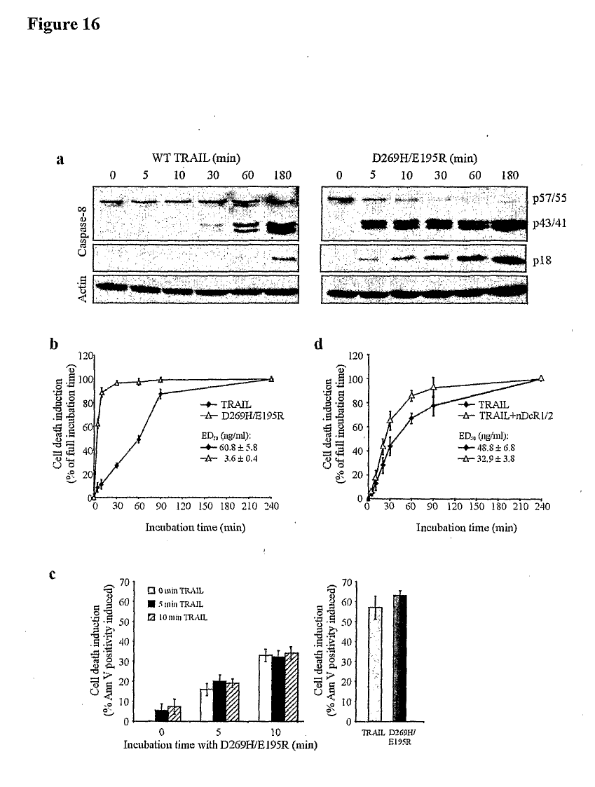

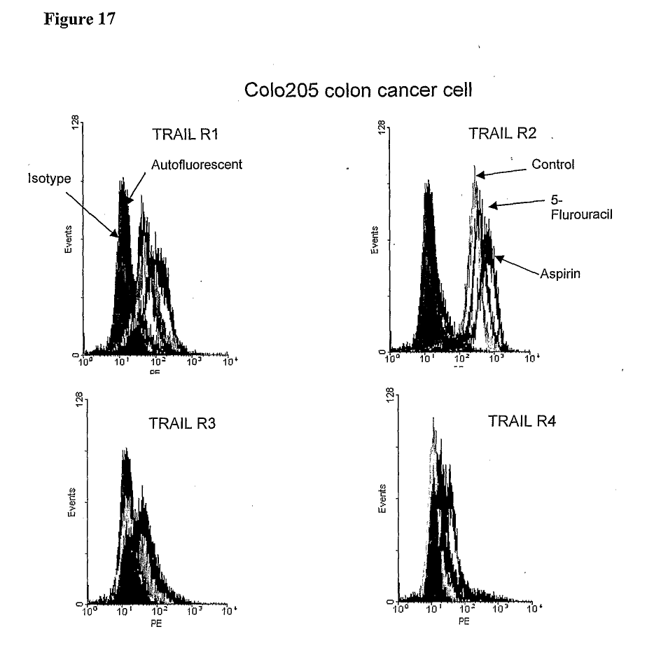

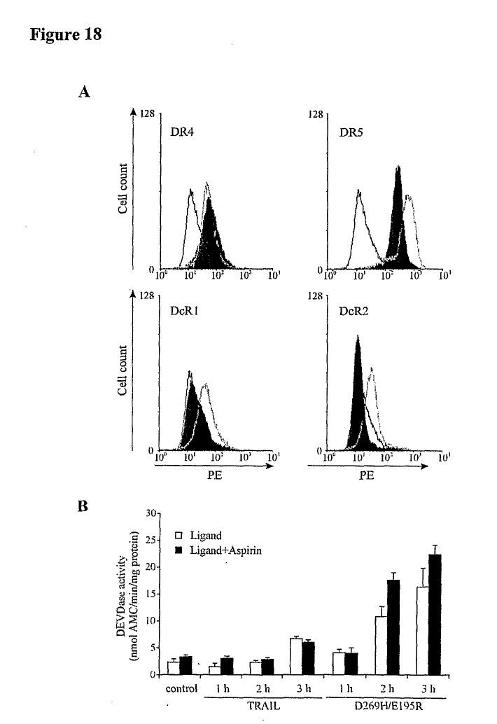

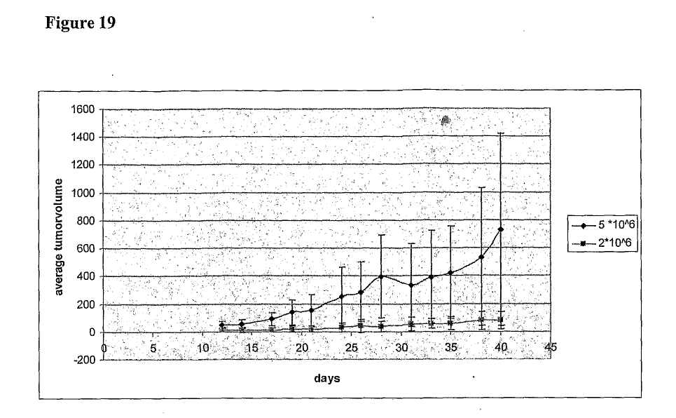

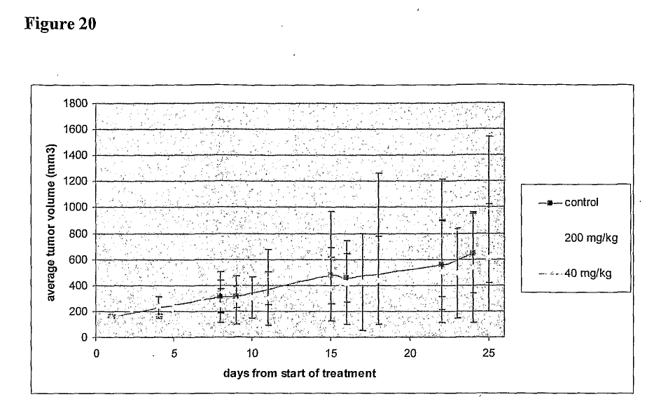

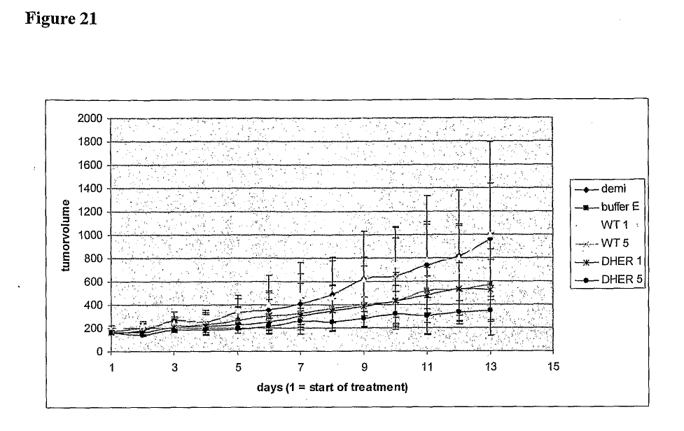

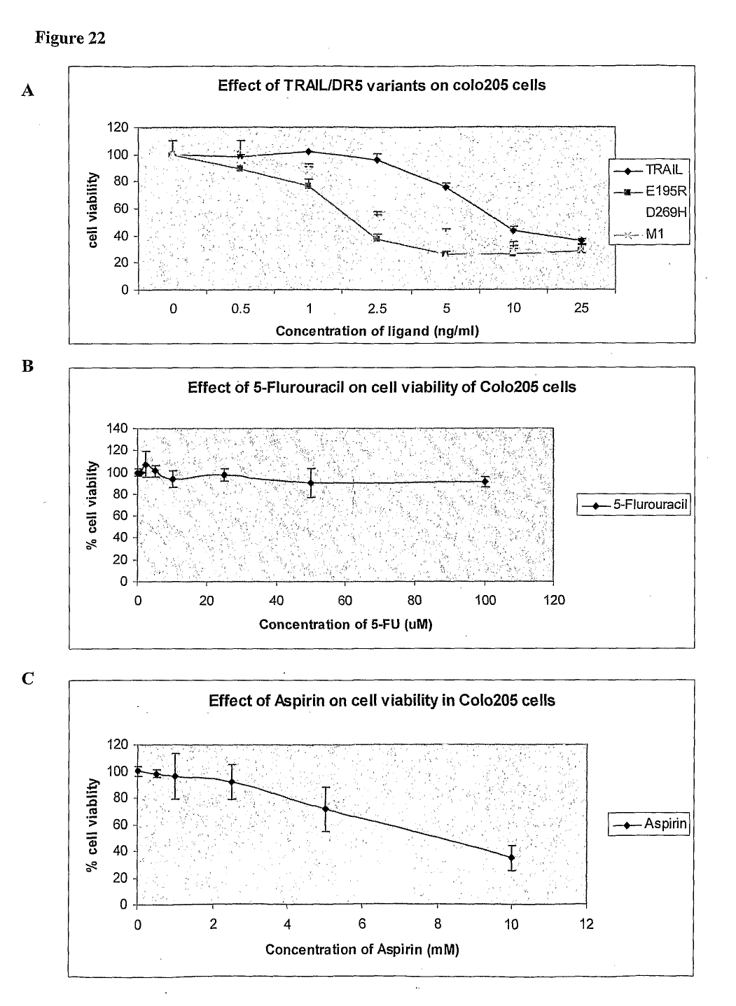

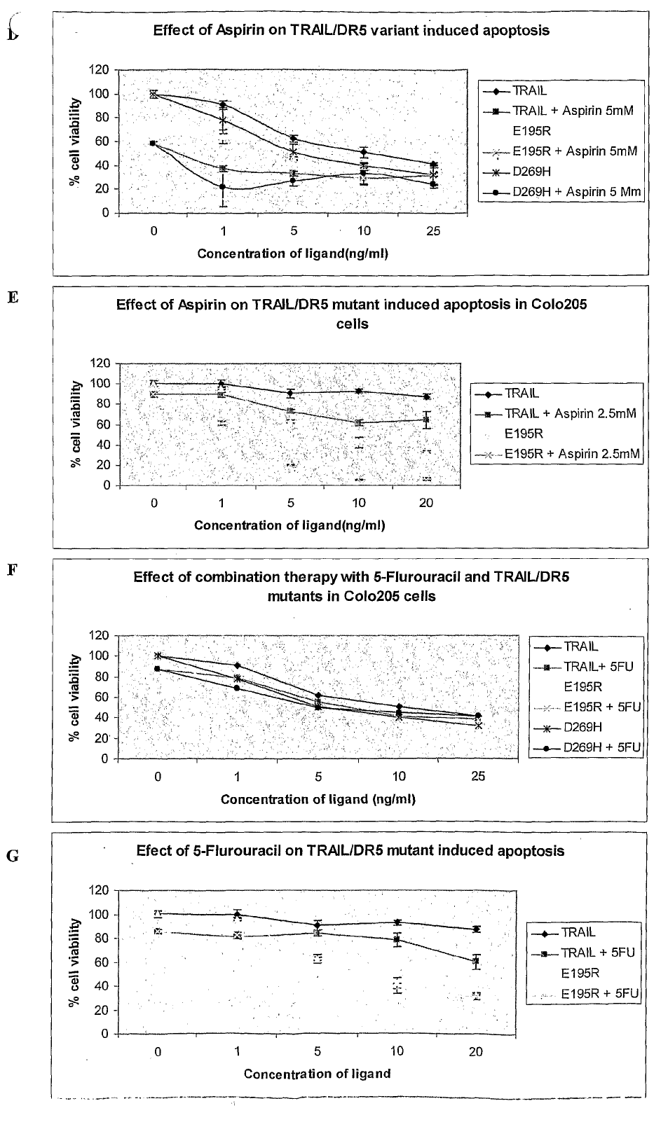

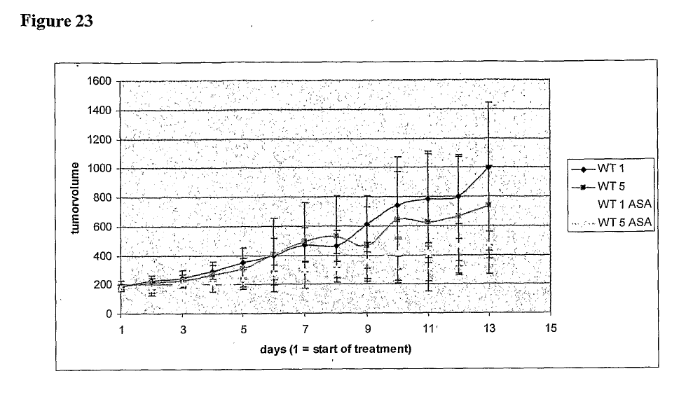

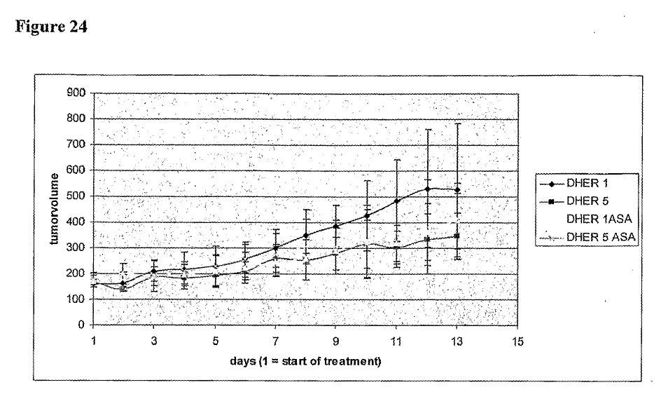

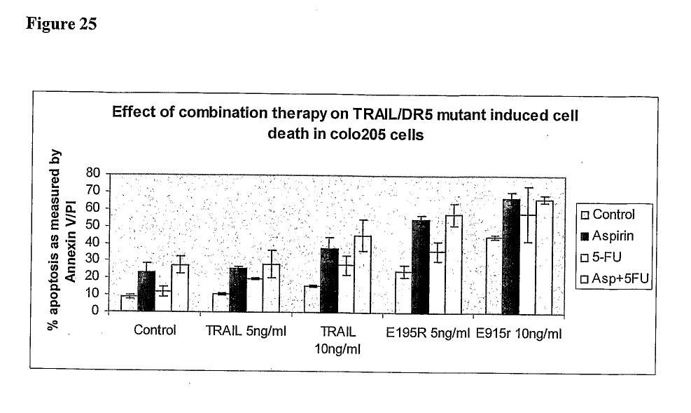

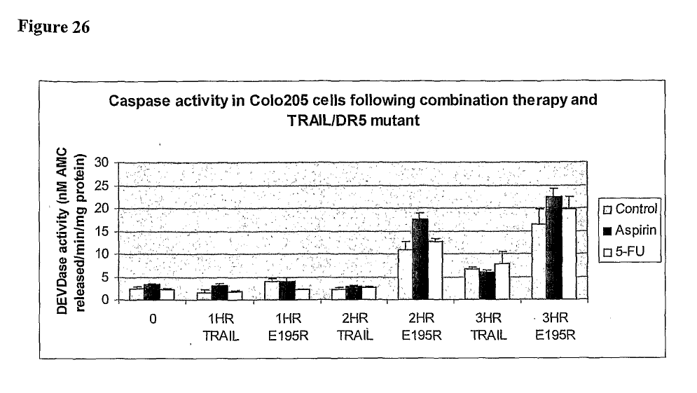

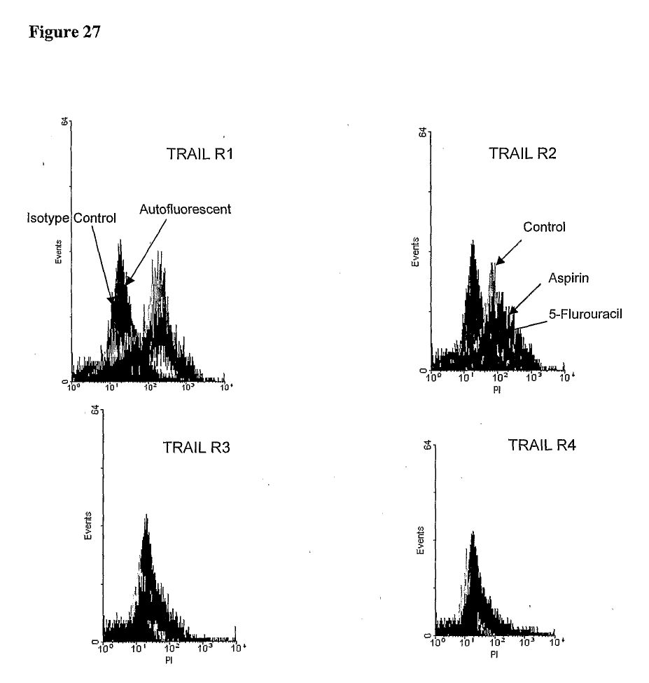

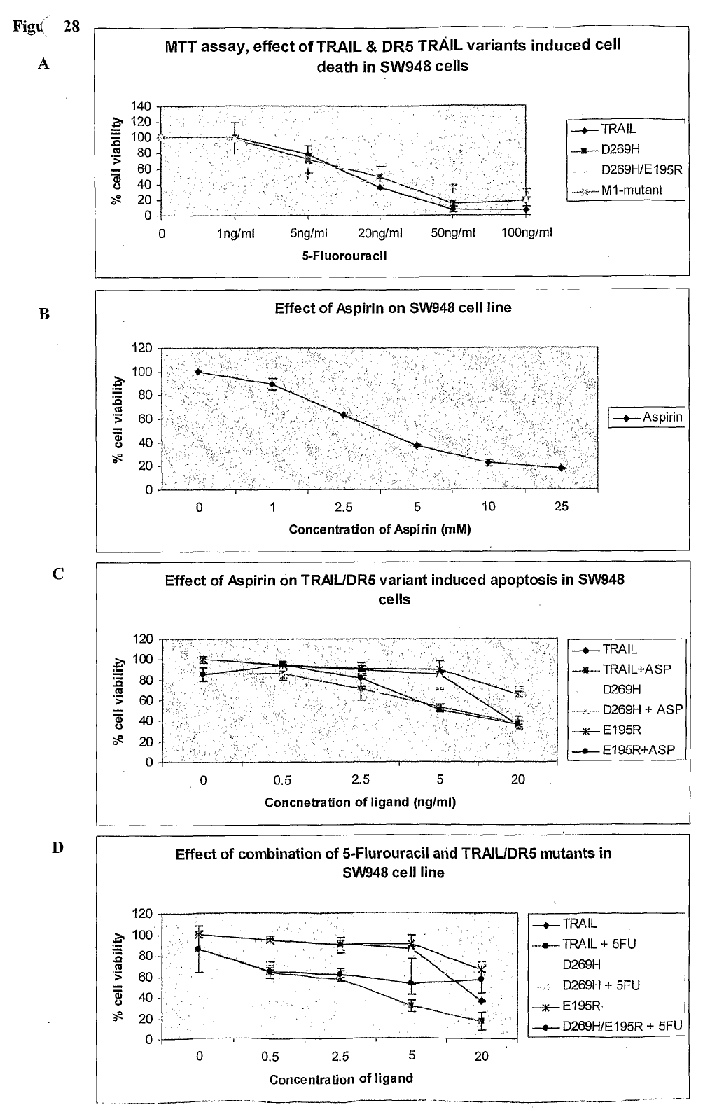

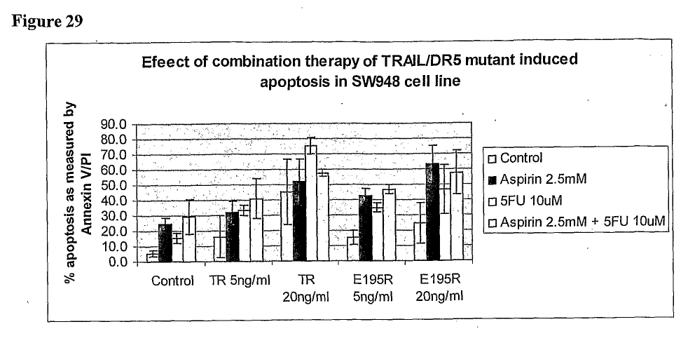

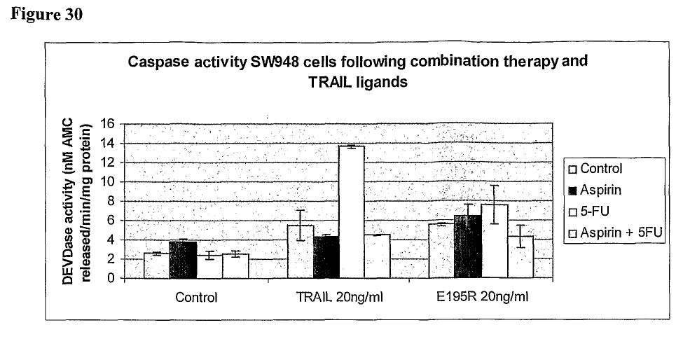

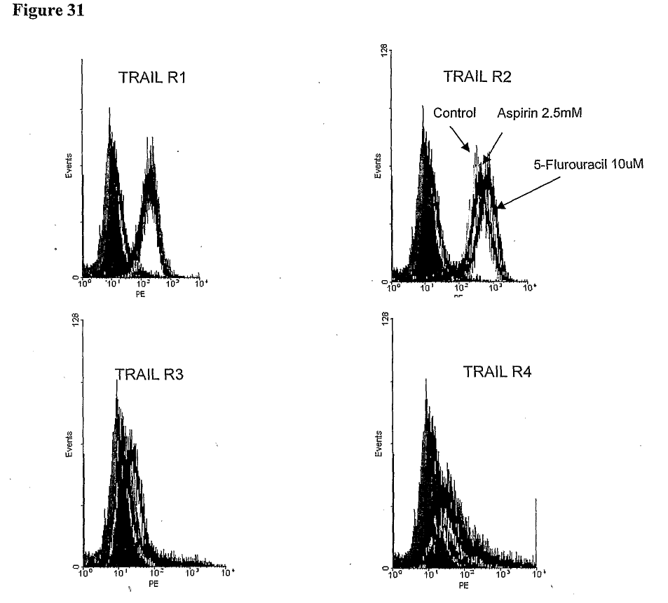

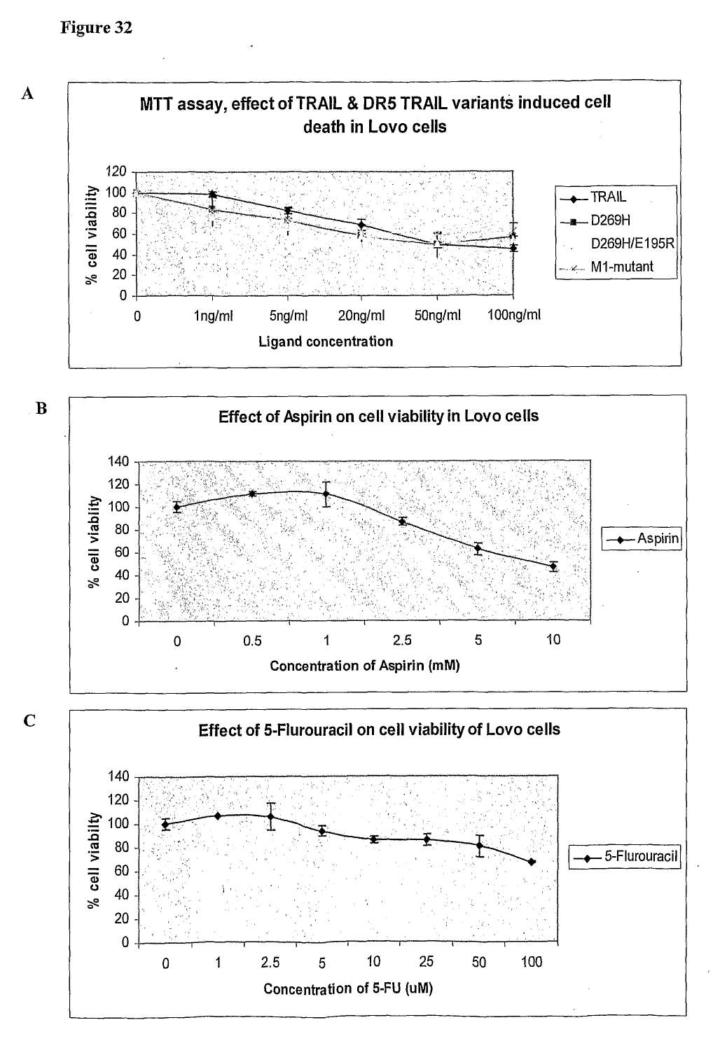

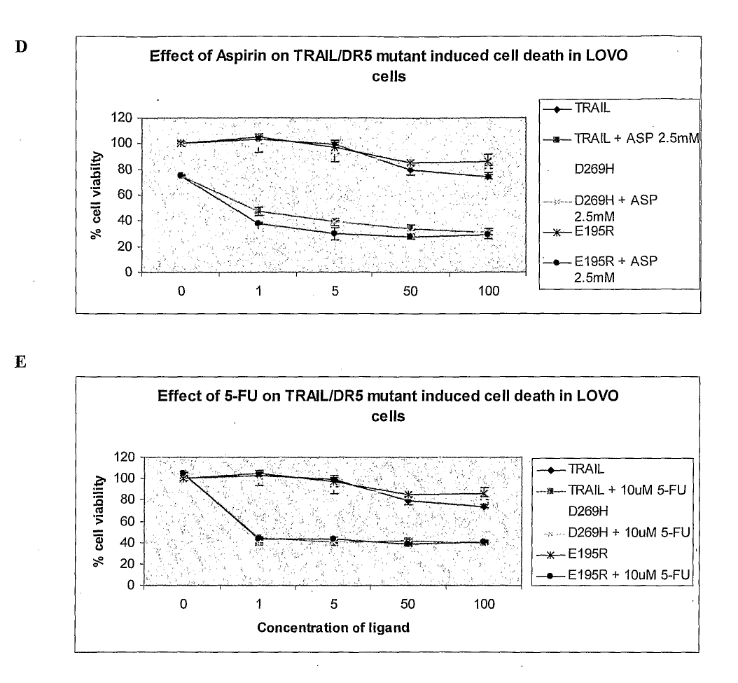

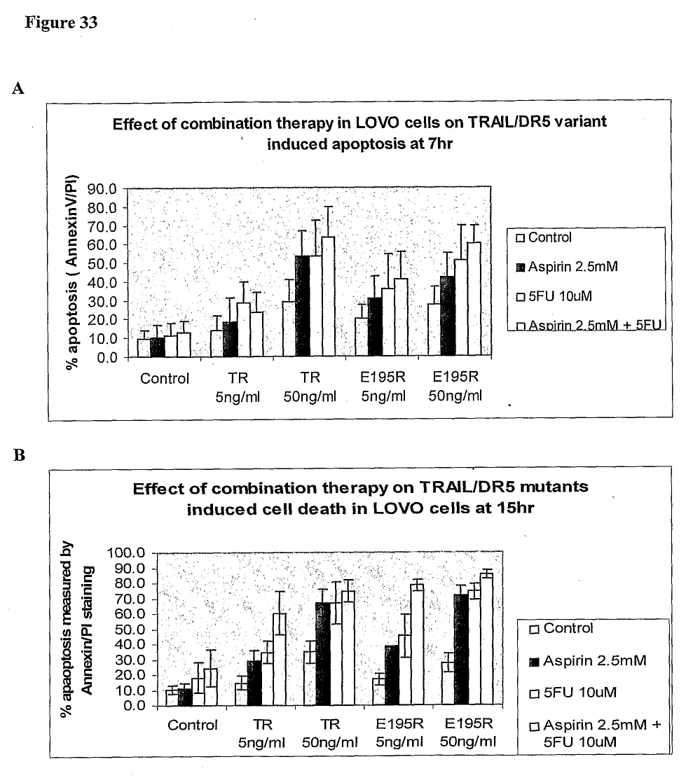

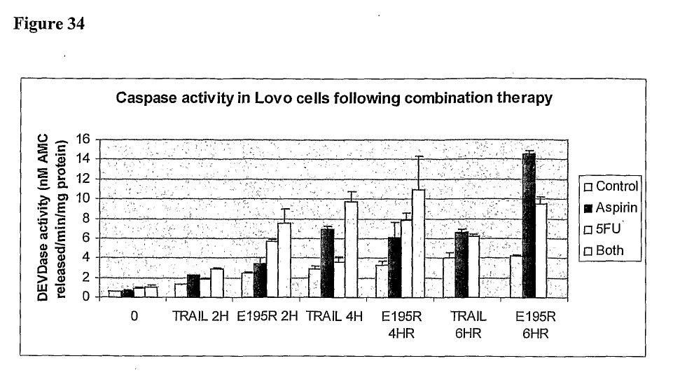

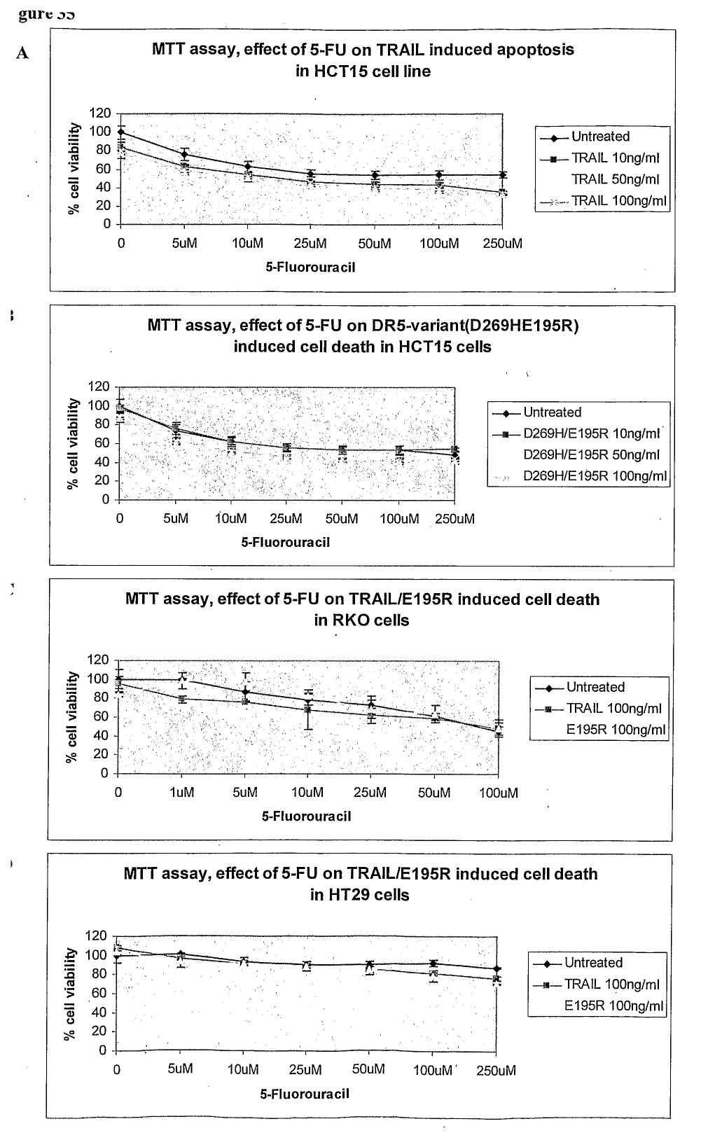

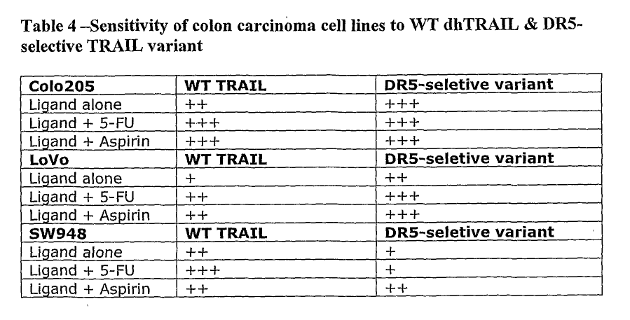

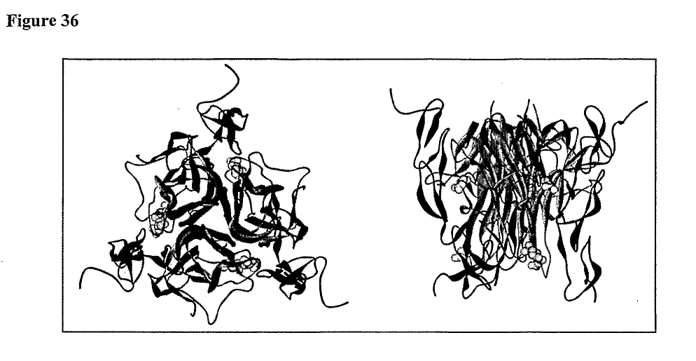

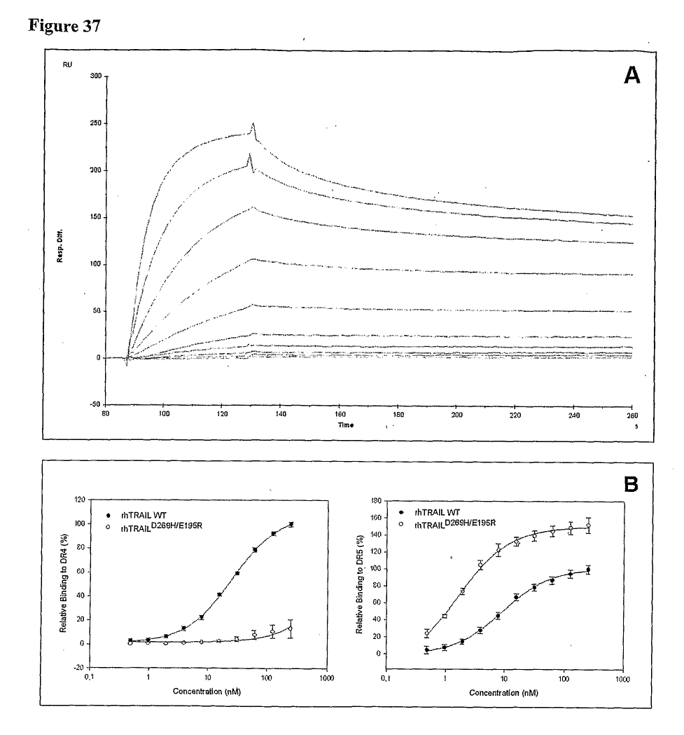

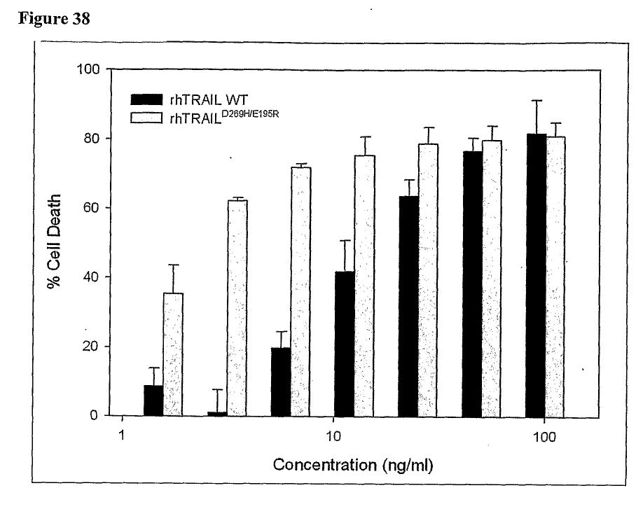

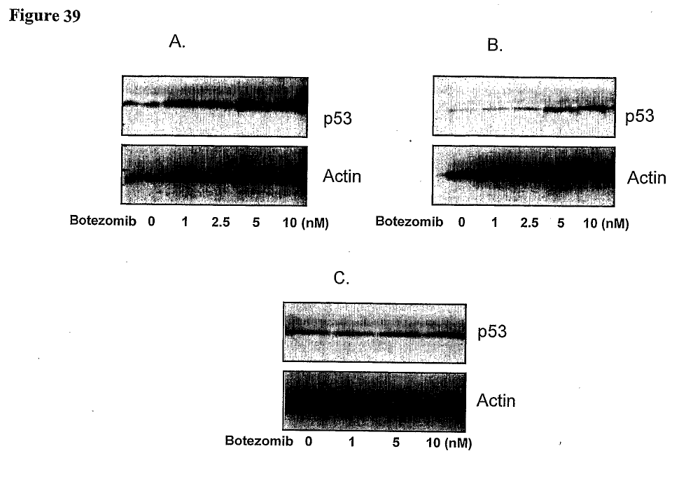

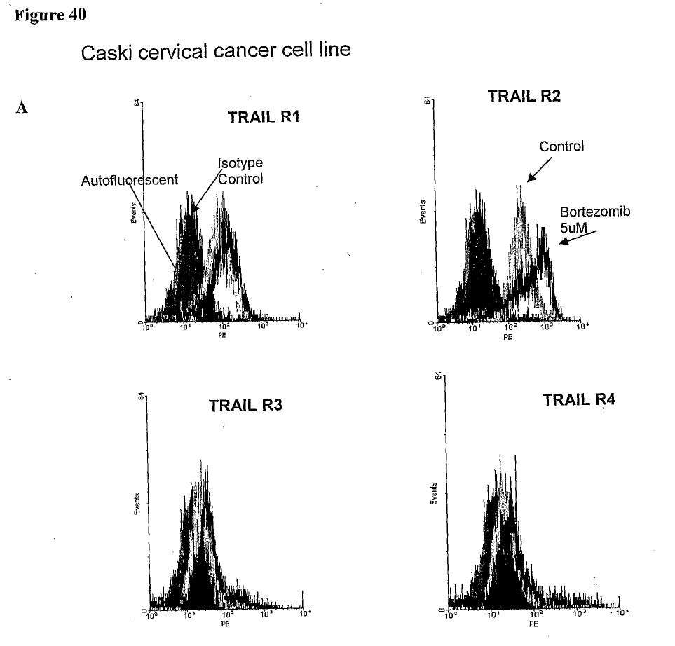

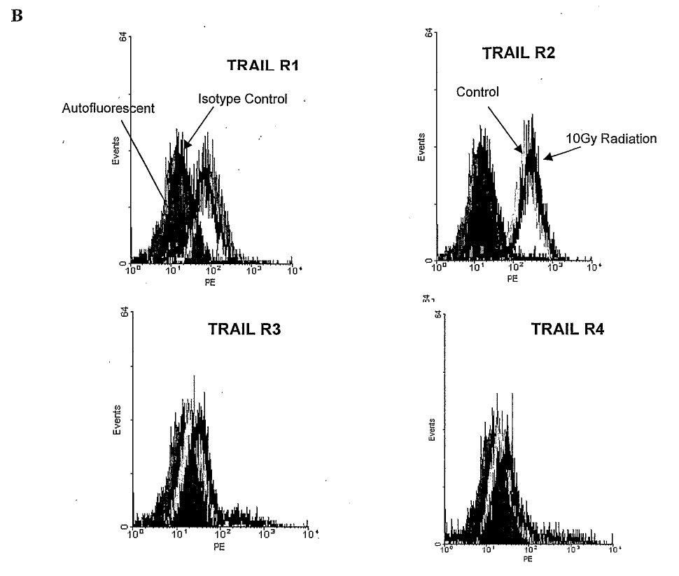

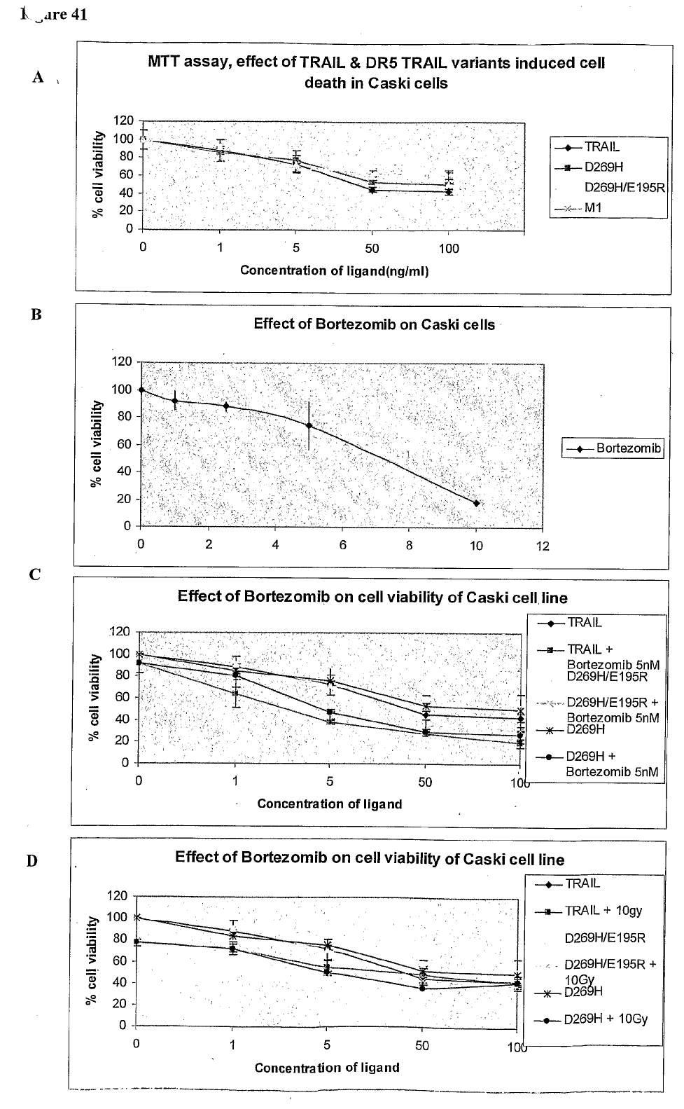

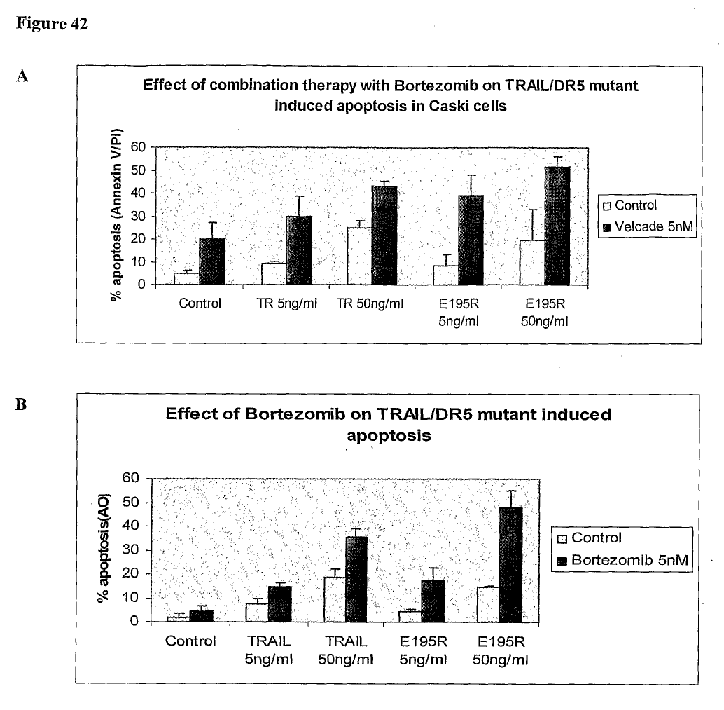

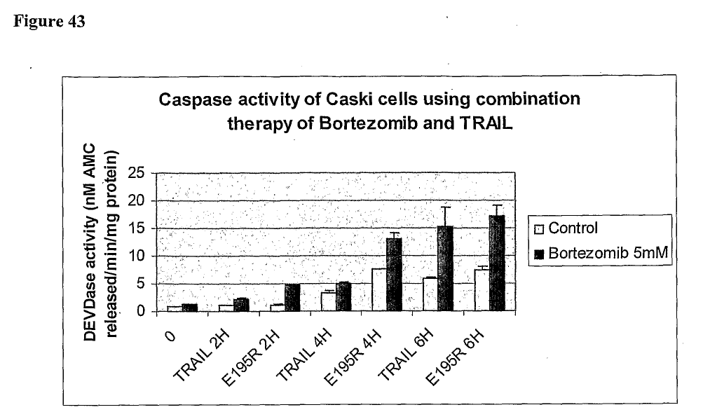

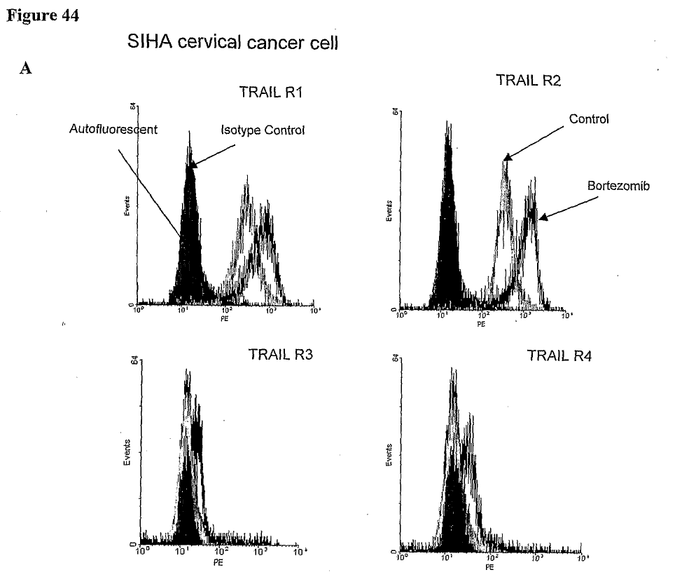

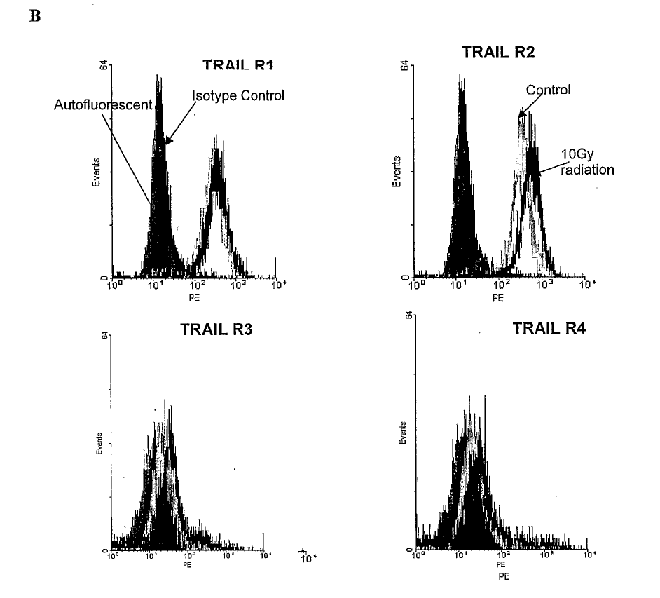

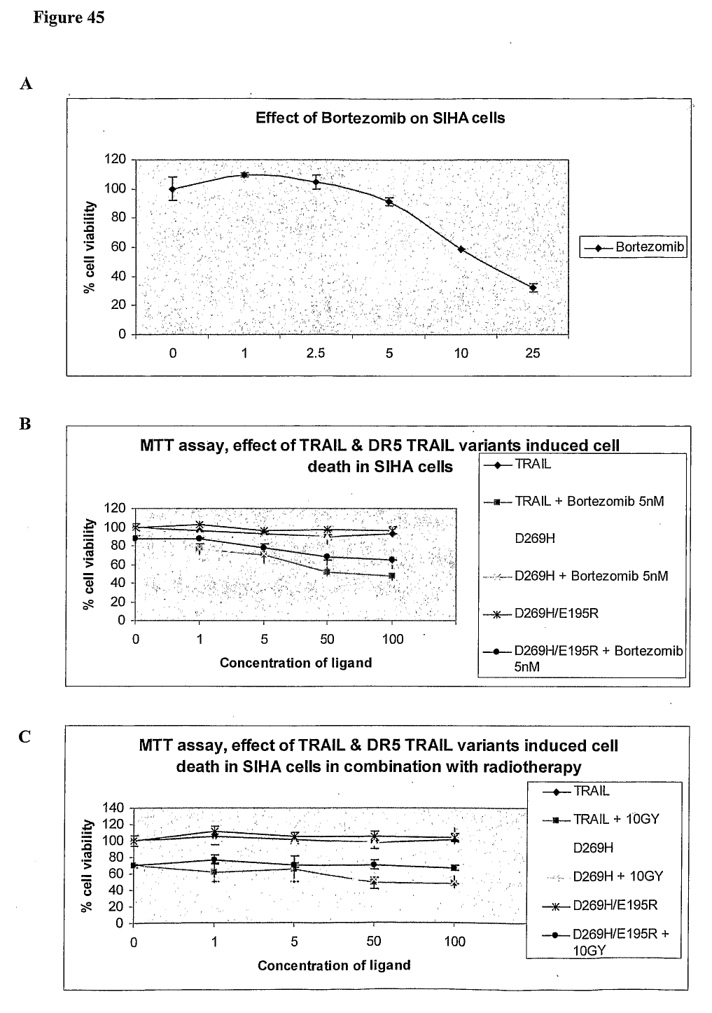

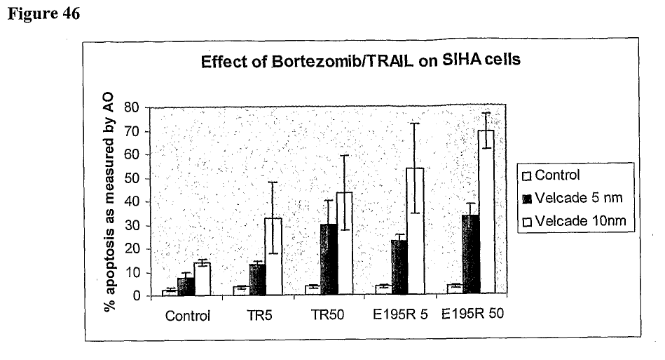

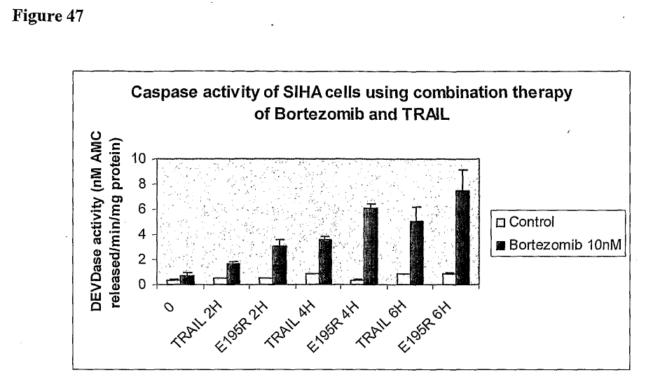

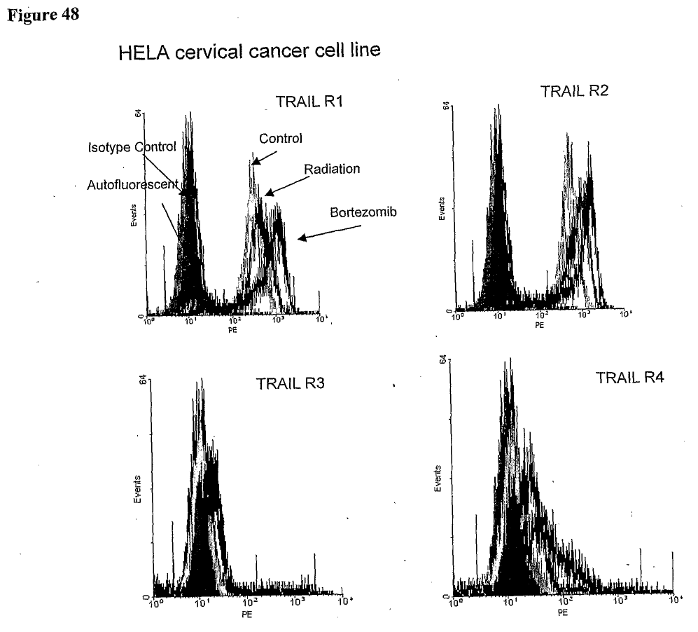

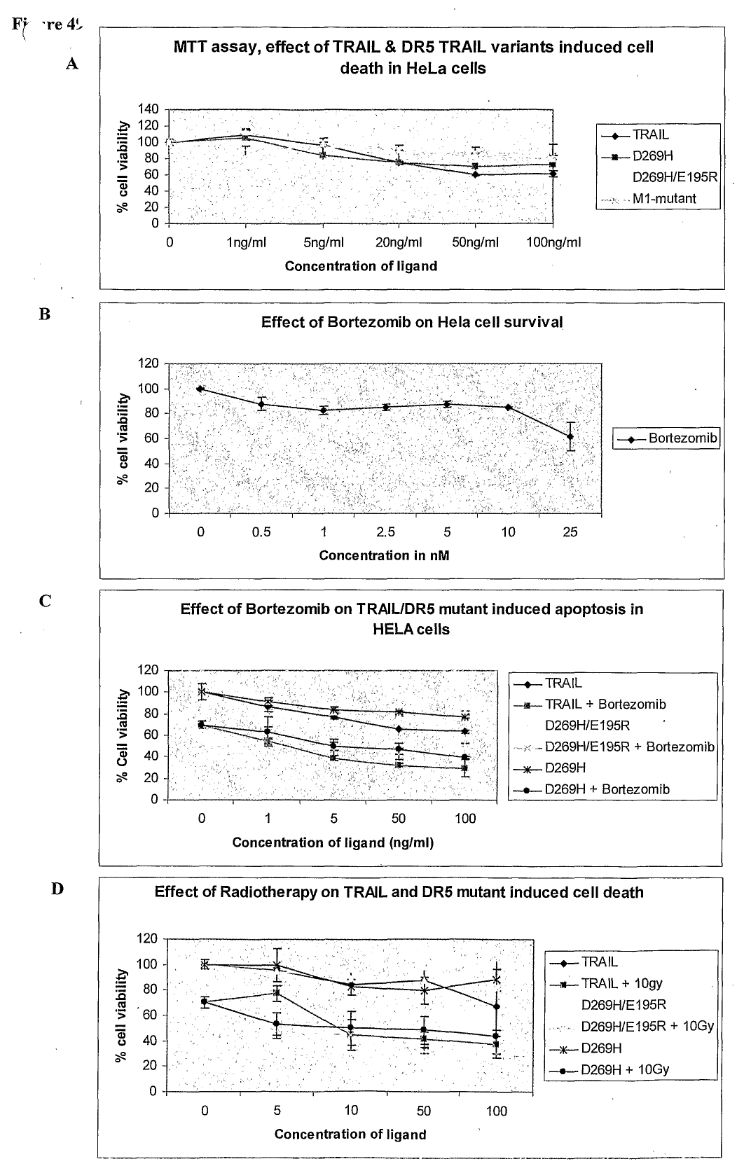

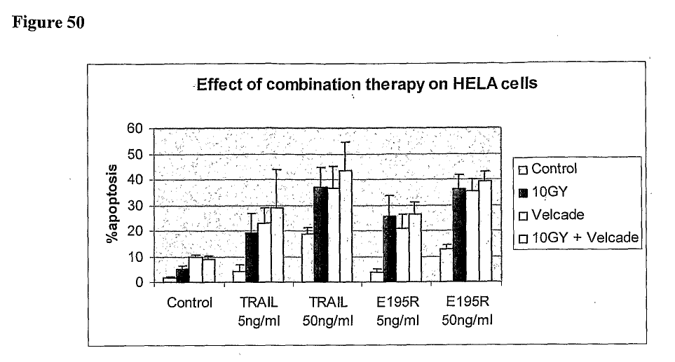

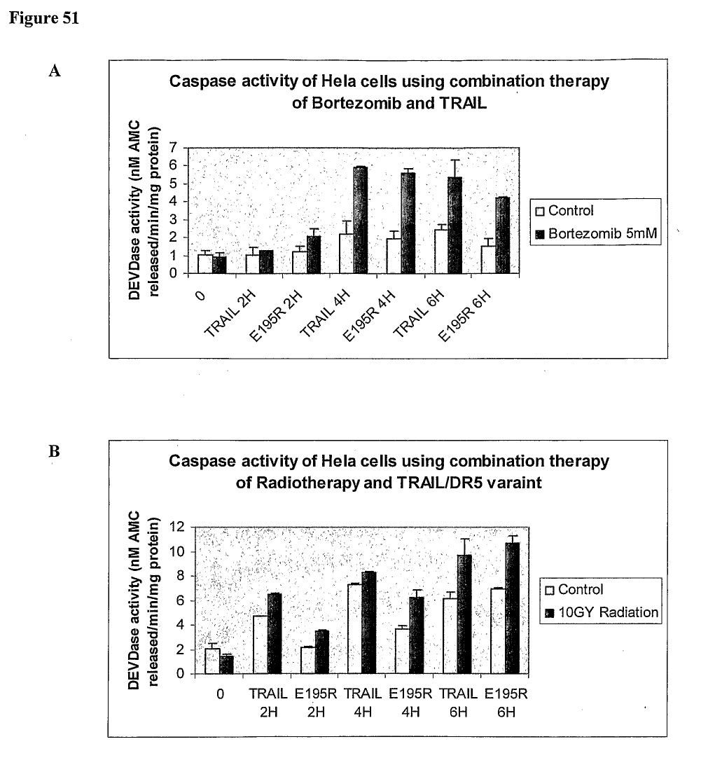

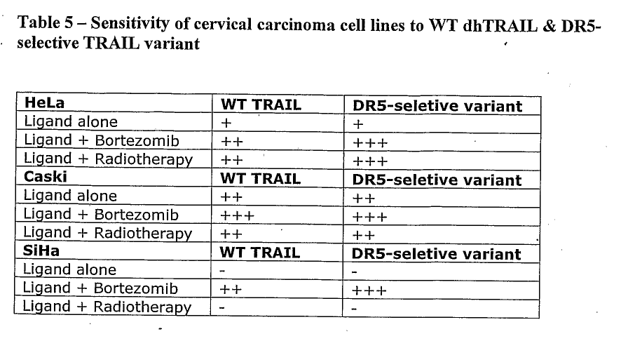

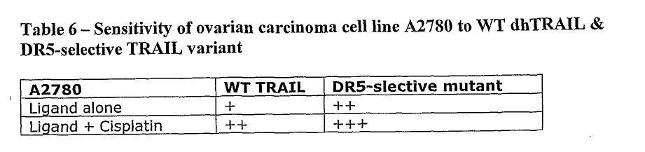

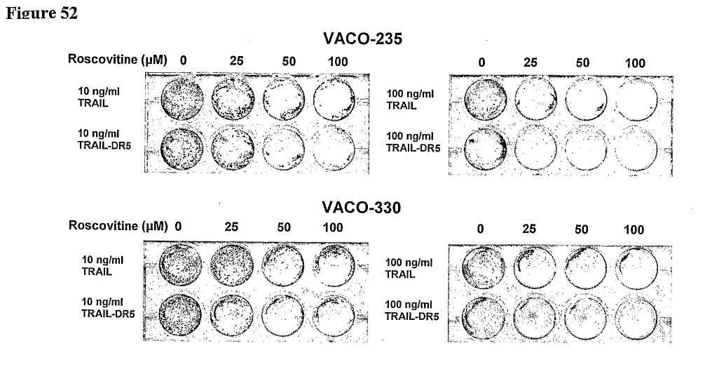

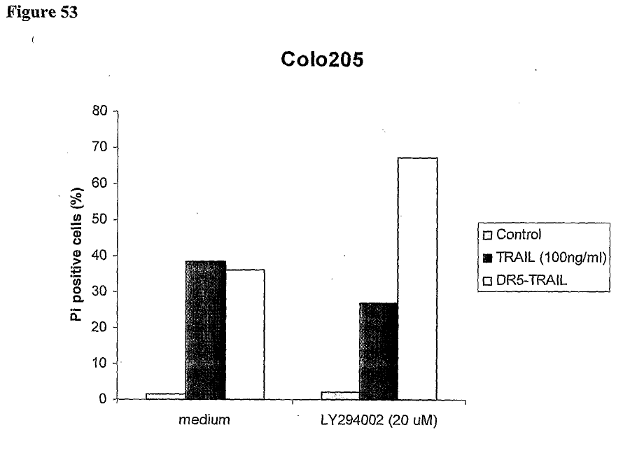

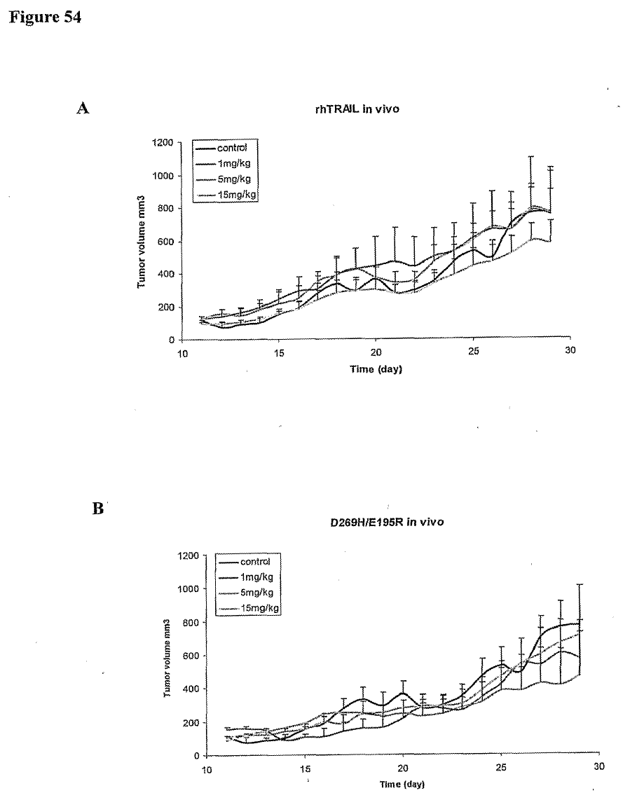

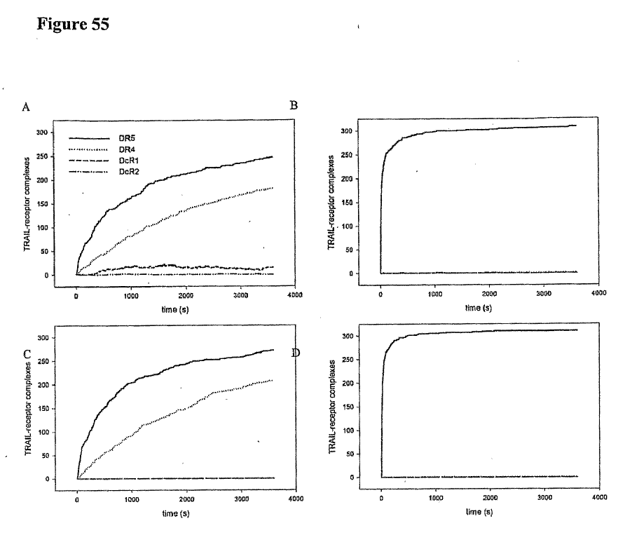

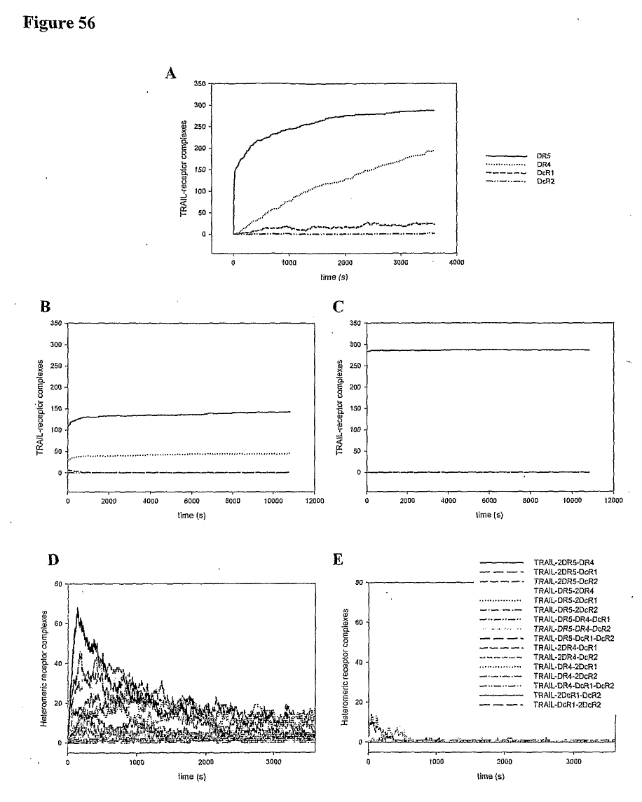

A2780 is a human ovarian cancer cell line, which expresses DR5 and low levels of DcR2 at its cell surface, whereas DR4 and DcR1 are undetectable (FIG. 1A). Treatment with cisplatin resulted in a dose dependent upregulation of DR5 and DcR2 expression (FIG. 1A). Long term exposure (96 h) of A2780 cells to low concentrations of TRAIL and TRAIL-DR5 induced a dose dependent loss of viability (FIG. 1B). Pre-incubation with low dose cisplatin (2.5 μM) prior to continuous treatment with TRAIL or TRAIL-DR5 caused a further drop in cell survival (FIG. 1C). TRAIL-DR5 decreased cell survival more effectively than TRAIL, both as a single agent (p<0.01) and in combination with cisplatin (p<0.001). Consistent with these results, short term exposure to TRAIL or TRAIL-DRS induced apoptosis, as determined by caspase-3 activity. Caspase activity was enhanced in all conditions upon pre-incubation with 2.5 μM of cisplatin (FIG. 2A). Combination of cisplatin with TRAIL-DR5 was more effective than combined exposure to cisplatin and TRAIL (p<0.001). Similar results were obtained with an acridine orange apoptosis assay (FIG. 2B). The apoptosis assays for TRAIL and TRAIL-DR5 were also performed with co-incubation of a DcR2 blocking antibody. Blocking of DcR2 did not enhance apoptosis induction by TRAIL or TRAIL-DR5 (FIG. 2C).

The involvement of the DR5 receptor in inducing apoptosis in these cells was further assessed in cells in which the DR5 receptor had been knocked down by RNA interference. To this end, A2780 cells were transfected with siRNA against DR5 (5′GACCCUUGUGCUCGUUGUC-dTdT3′ (sense) and 5′GACAACGAGCACAAGGGUC-dTdT3′ (anti-sense)) or luciferase, as a control or remained untransfected. 24 h after transfection the cells were exposed for 4 h to 2.5 μM, 10 μM and 30 μM cisplatin, after which all cells including those unexposed to cisplatin, were washed. The next day the cells were harvested and analyzed for DR5 expression by flow cytometry. DR5 expression is represented as mean fluorescence intensity (MFI). The data show that the DR5 receptor was not upregulated in response to cisplatin whereas DR5 upregulation in the control transfected cells was similar to untransfected cells (compare FIG. 9A with FIG. 1A).

The authors then assessed the efficiency of TRAIL to induce apoptosis in the control and the DR5 siRNA cells. To this end, a small fraction of the siRNA treated cell suspension was plated in a 96-wells plate and apoptosis levels were determined with acridine orange apoptosis assays counting at least 200 cells. Data represent the mean±SD of at least three independent experiments. The results show that apoptosis in the control cells was increased, when compared to the untreated cells. However, the DR5 siRNA cells did not show increased apoptosis in response to TRAIL (FIG. 9B), even when it was administered together with cisplatin. Therefore, DR5 siRNA resulted in a complete inhibition of TRAIL-induced apoptosis and blocked sensitization by cisplatin of TRAIL-induced apoptosis in A2780.

These data show that the DR5 pathway is important for inducing apoptosis in ovarian cancer cells which supports the notion for using DR5-specific mutants for the treatment of this cancer.

To assess membrane expression of DR5 in response to treatment with TRAIL or DR5-selective TRAIL, the A2780-Luc cell line was generated as follows. The luciferase gene was excised from pGL3-basic (Promega, Madison, Wis.) with HindIll and XbaI restriction enzymes (Roche Applied Science, Almere, The Netherlands) and ligated into a pcDNA3 vector under the control of the cytomegalovirus promotor. A2780 cells were cultured to 70% confluency and transfected by incubation with 2.5 μg plasmid DNA and 5 μl Fugene6 (Roche) in 250 μl OptiMem (Invitrogen, Breda, the Netherlands). Two days after transfection, transfectants were selected by adding geneticin (1 mg/ml) (Roche Applied Science, Almere, The Netherlands). Stable transfectants were obtained with a clonogenic assay followed by subcloning of positive clones by limiting dilution. The cell lines were cultured in RPMI 1640 (Life Technologies Breda, the Netherlands), supplemented with 10% heat inactivated fetal calf serum (FCS) (Bodinco BV, Alkmaar, the Netherlands) and 0.1 M L-glutamine in a humidified atmosphere with 5% CO₂ at 37° C. Geneticin was added once a month to the A2780-Luc culture. Luciferase expression was regularly tested with the luciferase assay (#E1500, Promega, Leiden, The Netherlands) and the BioRad ChemiDoc XRS system (BioRad, Veenendaal, The Netherlands).

The microculture tetrazolium assay, performed as described earlier³⁶, was used to measure cytotoxicity. The cells were cultured in HAM/F12 and DMEM medium, supplemented with 20% FCS and 0.1 M L-glutamine. wtTRAIL and TRAIL-DR5 were produced as we have described earlier^{11, 20}. Binding capacity to DR4 and DcR1 is virtually absent for TRAIL-DRS, whereas affinity for DcR2 is reduced¹¹. Treatment consisted of continuous incubation with 0-100 ng/ml TRAIL-DR5 or wtTRAIL. In cell viability assays assessing combination treatment with cisplatin, the cells were pre-incubated for 4 h (h) with 2.5 μM cisplatin (inhibitory concentration 20%-IC20), before addition of 0-25 ng/ml TRAIL or TRAIL-DR5.

Caspase-3/7 activity was used as an early apoptosis marker. Caspase-3/7 activity was determined with a caspase-3/7 fluorometric assay (Zebra Biosciences, Groningen, The Netherlands). For the fluorometric detection of DEVDase activity, cells were plated in 6-wells plates and left to adhere overnight. The cells were exposed to 2.5 μM cisplatin for 4 h, after which cisplatin was washed away with PBS (6.4 mM Na₂HPO₄; 1.5 mM KH₂PO₄; 0.14 mM NaCl; 2.7 mM KCl; pH=7.2) and fresh medium was added to the cells. 20 h later 50 ng/ml TRAIL-DR5 or TRAIL were added for various times. Thereafter the cells were harvested with trypsin and washed twice with ice-cold PBS. Before performing the caspase-3 activity assay according to the manufacturer's protocol, protein content of the lysates was determined with Bradford analysis²¹.

The acridine orange staining served as a marker for end/stage apoptosis. For the apoptosis assay 10,000 cells were incubated in 96-well tissue-culture plates. The cells were exposed to 2.5, 10 or 30 μM of cisplatin for 4 h, after which they were washed with PBS twice and incubated in regular culture medium. Twenty hours thereafter, cells were incubated in regular culture medium without or with 100 or 250 ng/ml TRAIL-DR5 or TRAIL for an additional 4 h. The same procedure was performed in the presence of 2.5 μg/ml mouse anti-DcR2 antibody (R&D Systems, Oxon, UK), with the exception that 1 h pre-incubation with the blocking antibody preceded TRAIL-DR5 and wtTRAIL-incubation. With this anti-DcR2 antibody an enhanced pro-apoptotic effect of TRAIL was observed in Colo205 human colon carcinoma cells³⁹ (FIG. 14D). After drug incubation, acridine orange was added to each well to distinguish apoptotic cells from viable cells. Staining intensity was determined by fluorescence microscopy. Apoptosis was defined by the appearance of apoptotic bodies and/or chromatin condensation and expressed as the percentage of apoptotic cells counted in three fields containing minimally 300 cells.

To quantitatively express the efficacy of combination therapy (cisplatin plus TRAIL or TRAIL-DR5) compared to both agents alone, we calculated enhancement ratios for cell kill and apoptosis as follows: enhancement ratio =% induced by combination therapy/(% induced by cisplatin alone+% induced by ligand).

Analysis of TRAIL-receptor membrane expression was performed by FACS analysis as described previously³⁵. For death receptor expression after cisplatin exposure, cells were exposed for 4 h, washed with PBS and incubated for 20 h in regular culture medium, after which FACS analysis was performed. Cells were subsequently washed twice with cold PBS containing 2% FCS and 0.1% sodium azide and incubated with phycoerythrin (PE)-conjugated mouse monoclonal antibodies against DR4, DR5, DcR1 and DcR2. Mouse PE-labeled IgG1 and IgG2B were used as isotype controls. All PE-labeled antibodies were purchased from R&D systems (Oxon, UK). Membrane receptor expression was analyzed with Winlist and Winlist 32 software (Verity Software House, Inc., Topsham, Me.) and is shown as mean fluorescent intensity (MFI) of all analyzed cells.

The involvement of the DR5 in inducing apoptosis in these cells was further assessed in cells in which the DR5 had been knocked down by small interfering RNA (siRNA). A strong cisplatin concentration-dependent increase in DR5 surface expression as well as DRS cellular protein expression was observed in the luciferase siRNA treated cells, while DR5 siRNA resulted in complete downregulation of DR5 in the presence of cisplatin up to 30 μM (FIG. 2D & 2E). In addition, DR5 siRNA completely protected A2780 cells against TRAIL and TRAIL-DR5-induced apoptosis also in the presence of cisplatin (FIG. 2F). Pre-treatment with cisplatin strongly enhanced apoptosis and cytoxicity induced by TRAIL-DR5 or TRAIL, with the combination of cisplatin and TRAIL-DR5 being most effective.

SiRNAs specific for human DR5 were designed and synthesized by Eurogentec (Seraing, Belgium). The double-stranded siRNA specific for human DR5 was 5′GACCCUUGUGCUCGUUGUC-dTdT3′ (sense) and 5′GACAACGAGCACAAGGGUC-dTdT3′ (anti-sense). Double-stranded luciferase siRNA sequence was 5′ CUUACGCUGAGUACUUCGA-dTdT 3′ (sense) and 5′UCGAAGUACUCAGCGUAAG-dTdT 3′ (anti-sense). A2780 cells were transfected in 6-wells plates (at 30-50% confluency) with siRNA duplexes (133 nM) using Oligofectamine transfection reagent according to the manufacturer's instructions (Invitrogen-Life Technologies). After 24 h, media was aspired and cells were harvested and plated. Then, cells were exposed to various cisplatin concentrations for 4 h, washed with PBS and incubated for 20 h in regular culture medium. Finally, cells were harvested and used for flow cytometry or Western blotting. For the apoptosis assay, cells were incubated in regular culture medium without or with TRAIL-DR5 or TRAIL (100 ng/ml) for an additional 4 h and apoptosis was determined with the acridine orange assay.

For Western blotting, cells were washed in ice-cold PBS and lyzed in SDS sample buffer (4% SDS, 20% glycerol, 0.5 M Tris-HCl, pH 6.8 and 0.002% bromophenol blue) containing 10% 2-β-mercaptoethanol, by boiling for 5 min in a water bath. Proteins were separated on a SDS-polyacrylamide gel and transferred to a polyvinylidene difluoride membrane (Millipore BV, Etten-Leur, the Netherlands) by wet blotting. Western blotting was performed using skim milk as blocking agent³⁶. The following antibodies were applied: rabbit anti-DR5 antibody from Cell Signaling Technology (Leusden, the Netherlands) and mouse-anti-actin as the control for equal protein loading from ICN Biomedicals (Zoetermeer, the Netherlands). Secondary antibodies conjugated with horseradish peroxidase (HRP) were obtained from DAKO Cytomation (Glostrup, Denmark). Chemiluminescence was detected using BM Chemiluminescence Blotting Substrate (POD) or Lumi-LightPLUS Western blotting substrate Roche Diagnostics (Almere, the Netherlands).

### Example 3

### ¹²⁵I-TRAIL Biodistribution In Tumor-Bearing Mice

Tissue biodistribution and tumor uptake of intravenously (IV) and intraperitoneally (IP) administered ¹²⁵I-TRAIL were compared in nude mice with IP A2780-Luc xenografts.

Radioiodination of TRAIL was performed with a TRAIL solution of 1 mg/ml in a pH 7.4 TRIS buffer, containing 100 μM zinc sulphate and 10% glycerol. 45 μg TRAIL and 50 μg chloramine T (Merck, Amsterdam, The Netherlands) were allowed to react with 70 MBq ¹²⁵I-NaI in 0.05 M NaOH (pH 9.0, GE Healthcare, Eindhoven, The Netherlands) during 3 min at pH 8.0. The labeling reaction was terminated with sodium metabisulfite (Acros Organics, Geel, Belgium). Non-bound ¹²⁵1 was removed by gel filtration chromatography. The PD-10 column (Sephadex™ G-25M, Amersham Biosciences AB, Uppsala, Sweden) was eluted with TRIS buffer, containing 100 μM zinc sulphate, 10% glycerol and 0.5% human serum albumin. Nude mice with IP A2780-Luc xenografts were prepared, as described above.

The in vivo biodistribution study was conducted in 50 mice after establishment of A2780-Luc IP xenografts. ¹²⁵I-TRAIL (0.15 ml; 150 kBq, 0.5 μg) was administered IV through retro-orbital injection in 25 mice, and IP in 25 mice. At five time points after injection (t=15, 30, 60, 90 and 360 min) groups of 5 mice were sacrificed and organs and tissues were excised, rinsed for residual blood and weighed. Tumor tissue was additionally fixed in 10% buffered formalin for histological assessment. Samples were counted for radioactivity in a calibrated well-type LKB-1282-CompuGamma counter. Tissue activity was expressed as percentage of the injected dose/g tissue (% ID/g). Tumor-to- blood and tumor-to-muscle ratios were also calculated. All data were corrected for physical decay and compared with a known standard sample. Pharmacokinetic parameters were derived using the KINFIT module of the MW/PHARM computer program package (version 3.50, MediWare, Groningen, The Netherlands). Clearance rates of ¹²⁵I-TRAIL from the circulation were calculated using non-linear regression analysis.

The administration route influenced the disposition of ¹²⁵I-TRAIL. Blood activity (% ID/g) was higher at 15 (43.29±11.04 vs. 25.30±5.04) and 30 min (30.51±12.40 vs. 15.33±3.78) after IV vs. IP injection, whereas it was lower at 90 (7.52±1.22 vs. 23.74±6.85) and 360 min (2.63±0.56 vs. 8.26±1.74). The blood kinetics of ¹²⁵1-TRAIL in blood could be described by a two-compartment model. The resulting blood activity vs. time profiles (FIG. 3A) showed a higher area under the time curve (AUC) after IP administration (89.8) than after IV administration (53.7). After IP injection the peak blood activity is lower than after IV injection, but remains higher for a longer period in time. Kidney uptake (% ID/g) showed the same pattern as blood pool activity, with higher activity after IV administration vs. IP administration at 15 (199.2±40.69 vs. 19.83±1.38) and 30 min (126.6±49.68 vs. 21.45±1.90) and lower activity at 90 (12.73±2.47 vs. 17.87±1.28) and 360 min (2.06±0.56 vs. 4.45±0.31). Activity in well-perfused organs such as the lungs, liver and spleen displayed similar kinetics as the blood pool activity in both administration routes. Stomach activity increased over time, which can be attributed to in vivo dehalogenation. IP administration resulted in high tumor activity at 15 min (11.31±1.51) and 60 min (12.91±3.29) with a gradual decrease to 360 min, whereas after IV administration initial low tumor activity increased to a maximum at 60 min (6.85±1.29 IV). The tumor to blood ratios were higher after IP administration at 15 (0.13±0.02 vs 0.48±0.03) and 60 min (0.38±0.04 vs. 0.55±0.06). Tumor to blood ratios remained constant over time after IP injection and increased over time after IV administration (FIG. 3B).

To determine whether the higher tumor uptake after IP administration resulted in enhanced efficacy of ¹²⁵I-TRAIL, paraffin embedded tumor tissue obtained at 15 and 360 min after ¹²⁵I-TRAIL injection was stained for cleaved caspase-3. While almost no cleaved caspase-3 was detected in samples obtained at 15 min (FIG. 4A, B), tissue obtained at 360 min showed increased cleaved caspase-3 staining. IP administration resulted in caspase-3 activation near the surface of the tumor and near small blood vessels (FIG. 5A), whereas IV administration induced detectable caspase-3 activity near blood vessels but not near the tumor border (FIG. 5B).

The rationale behind IP drug administration is to increase local drug exposure, while lowering plasma clearance. We show that IP TRAIL-administration resulted in a higher area under the curve and a reduced clearance. The high kidney activity confirms the function of the kidney as main site of TRAIL-clearance, which is not influenced by IP administration. Activity in most organs followed that of blood pool activity, suggesting that distribution to normal tissues was limited.

The inventors have also shown that specific tumor retention of ¹²⁵I-TRAIL takes place. It was demonstrated by maximal tumor activity at 60 min after IV administration, while activity in all other well perfused organs is maximal at 15 min. IP administration resulted in even higher activity in the tumor and a higher cumulative tumor to blood ratio, which shows that IP TRAIL administration results in higher tumor drug exposure compared to IV administration in this model. Moreover, 6 h after IP injection of a low dose of TRAIL, cleaved caspase-3 was detected in the superficial layers of the tumors, suggestive of TRAIL penetration by free-surface diffusion. This is a possible advantage of TRAIL and -variants over monoclonal antibodies, which show limited IP tumor penetration after IP administration.

### Example 4

### Cell Killing of Ovarian Carcinoma Cell Line A2780 By wtTRAIL And DR5-Selective TRAIL

The ovarian carcinoma cell line A2780 expresses very low levels of DR4, and high levels of the other three membrane-bound TRAIL receptors. Sensitivity of these cells to wild type TRAIL and the DR5-specific variant was compared initially as a single agent. The sensitivity pattern is illustrated in the Table 6, with + indicating minimally sensitive, ++ moderately sensitive and +++ very sensitive.

A2780 cells showed increased sensitivity to the DR5-selective variant compared to wild type TRAIL. These effects were enhanced when the ligands were used in combination with Cisplatin chemotherapy, which is currently the active agent used in the treatment of ovarian carcinoma. Again, the DR5-selective variant showed higher efficacy than wtTRAIL.

### Example 5

### In Vivo Efficacy of TRAIL, TRAIL-DR5 And Cisplatin On IP Xenografts Determined By Bioluminescence Imaging

The human ovarian cancer cell line A2780 forms IP xenografts mimicking peritonitis carcinomatosis in nude mice. The A2780-Luc cell line was generated with HindiII and XbaI, restriction enzymes (Roche Applied Science, Almere, The Netherlands). The luciferase gene was excised from pGL3-basic (Promega, Madison, Wis.) and ligated into a pcDNA3 vector under the control of the cytomegalovirus promotor. A2780 cells were cultured to 70% confluency and transfected by incubation with 2.5 μg plasmid DNA and 5 μl Fugene6 (Roche) in 250 μl OptiMem (Invitrogen, Breda, the Netherlands). Two days after transfection, transfectants were selected by adding geneticin (1 mg/ml) (Roche Applied Science, Almere, The Netherlands). Stable transfectants were obtained with a clonogenic assay followed by subcloning of positive clones by limiting dilution. The cell lines were cultured in RPMI 1640 (Life Technologies Breda, the Netherlands), supplemented with 10% heat inactivated fetal calf serum (FCS) (Bodinco BV, Alkmaar, the Netherlands) and 0.1 M L-glutamine in a humidified atmosphere with 5% CO₂ at 37° C. Geneticin was added once a month to the A2780-Luc culture. Luciferase expression was regularly tested with the luciferase assay (#E1500, Promega, Leiden, The Netherlands) and the BioRad ChemiDoc XRS system (BioRad, Veenendaal, The Netherlands).

Female nude mice (Hsd:Athymic Nude-nu) were obtained from Harlan Nederland (Horst, The Netherlands) at 6-8 weeks of age (˜21 g). Inoculation was performed after 10 days of acclimatization. All animal studies were conducted in accordance with the Law on Animal Experimentation and local guidelines, and were approved by the local ethical committee.

Imaging was conducted with the IVIS 100 series (Xenogen Corporation Alameda, Calif.), composed of a cooled charge-coupled device camera connected to a light tight black chamber. Before in vivo imaging animals were anesthetized with 4% isoflurane and injected IP with D-luciferin (150 mg/kg, Xenogen) reconstituted in sterile PBS. Mice were placed in prone position on a warmed stage (37° C.) in the imaging chamber, and grayscale reference images were obtained under dim illumination. Pseudocolor images representing bioluminescent intensity were acquired with Livinglmage software (version 2.50, Xenogen) 10 and 15 min after D-luciferin injection in complete darkness. These images were superimposed on the grayscale images for analysis with Igor Pro software (version 4.09, WaveMetrics, Lake Oswego, Oreg.). All bioluminescence imaging (BLI) data are depicted in radiance units (photons/sec/cm²/sr) enabling absolute comparisons between bioluminescent images and represent final data obtained after subtraction of the background signal.

The response of IP A2780-Luc xenografts to treatment with TRAIL, TRAIL-DRS and cisplatin or a combination of either TRAIL or TRAIL-DRS with cisplatin was assessed. Tumor regression was not visible within the first 48 h after treatment initiation at day 5, but was clearly evident at the end of the first treatment period (day 9), with the largest signal reduction seen after combination of TRAIL or TRAIL-DRS with cisplatin (FIG. 6A). Signals rose in the days between both treatments. All treatment groups except the TRAIL-treated arm had significantly smaller tumors at day 16 than the vehicle treated group. This is reflected in the mean signal reduction as to vehicle treated mice; whereas TRAIL therapy did not result in a significant decrease (48.8%−range 32.8−64.6%, p=0.097); TRAIL-DR5 and cisplatin gave a reduction of 68.3% (range 61.8−74.8%, p=0.015) and 72.3% (range 59.8−84.9%, p=0.009) respectively. Combination therapies were highly effective; TRAIL combined with cisplatin caused a decline in signal intensity of 84.8% (range 73.5−96.1, p=0.003) and the combination of TRAIL-DRS with cisplatin resulted in 96.5% (range 93.7−99.4%, p=0.002) signal reduction. Thus, all therapies exhibited significant anti-tumor activity at the end of treatment, with the combination therapies displaying the highest activity. The decline in signal intensity after combination therapy of TRAIL-DR5 with cisplatin was higher than the mean light reduction after cisplatin therapy alone (p=0.027).

In general, light intensity at the end of treatment was inversely associated with survival (FIG. 6B). Animals were sacrificed when a bioluminescent signal ≧3.1×10⁸ photons/sec/cm²/sr was reached. The median survival of the vehicle controls was 16 days, with no mice surviving after 18 days. Monotherapy with TRAIL and TRAIL-DR5 prolonged median survival to 21 days (p<0.001) and monotherapy with cisplatin to 28 days (p<0.0001). Combination of TRAIL-DR5 with cisplatin resulted in a median survival of 31 days (p<0.0001), and TRAIL combined with cisplatin in 32.5 days (p<0.0001). The latter was also significant as compared to cisplatin monotherapy (p=0.038). Liver histology at sacrifice did not show any signs of liver damage.

### Example 6

### Biological Activity of DR5-Specific Mutants

To assess the biological activity related to DR5 binding, various cancer cells were used. Colo205 colon carcinoma cells and ML-1 chronic myeloid leukemia cells express all four TRAIL receptors on the cell surface, as shown by using FACS analysis (FIG. 7), and are sensitive to TRAIL-induced apoptosis. To test the involvement of DR4 versus DR5 in TRAIL-induced cell death, Colo205 cells were treated with neutralizing anti-DR4 or anti-DR5 antibody for 1 h before the addition of TRAIL. Both antibodies reduced TRAIL-mediated cell death and had an additive effect when used in combination (FIG. 8A). However, the DR5-neutralizing antibody was 3× more effective than the DR4-neutralizing antibody, demonstrating that TRAIL-induced apoptosis in Colo205 cells is primarily mediated by DR5. In contrast, the DR4 pathway is the major mediator of TRAIL-induced apoptosis in ML-1 cells (FIG. 8A.) To examine whether the DR5-specific TRAIL variants induce cell death in Colo205 cells by way of the DR5 receptor, 1 μg/ml neutralizing anti-DR4 or -DR5 antibodies were administered 1 h before ligand treatment. The presence of the anti-DR4 antibody failed to prevent death induced by the DR5-specific variants. However, 1 μg/ml anti-DR5 antibody significantly reduced the amount cell death (FIG. 8B).

Colo205 and ML-1 cells were then treated with increasing concentrations of TRAIL or the DR5-specific variants D269H, D269HE195R, and D269H/T214R, and their cytotoxic potential was measured with a 3-(4,5-dimethylthiazol-2-yl)-2,5-diphenyl tetrazolium bromide (MTT) assay. In Colo205 cells, all TRAIL ligands were biologically active and induced cell death at levels that were either comparable with that of wild-type TRAIL or were up to 5-fold more active than wild-type TRAIL (FIG. 8C and Table 3). Contrary to Colo205 cells, only TRAIL was able to induce cell death in ML-1 cells (FIG. 8D).

Similar results were obtained by using EM-2 chronic myeloid leukemia cells expressing only the DR4 receptor and lacking the DR5 receptor and by using the ovarian cancer cell line A2780, which expresses DR5 but lacks DR4 on its surface and is relatively insensitive toward TRAIL-induced cell death. Although EM-2 cells were sensitive to TRAIL-induced cell death (50 ng/ml TRAIL initiating >80% cell death), treatment with any of the DR5 mutants failed to induce significant cell death (data not shown). In A2780 cells, however, the cytotoxic activity of D269H, D269HE195R, and D269HT214R is significantly increased, showing both an increased maximum response and drastically decreased EC50 values when compared with wild-type TRAIL (FIG. 8E and Table 3).

An additional experiment using D269HE195R in wild-type BJAB cells responsive to both DR4- and DR5-mediated cell death (BJABwt), BJAB cells deficient in DR5 (BJABDR5 DEF), and BJAB cells deficient in DR5 and stably transfected with DR5 (BJABDR5 DEF+DR5) confirm our findings. D269HE195R was able to induce cell death in BJABwt cells but was unable to induce significant cell death in BJABDR5 DEF cells when compared with wild-type TRAIL. In the DRS transfected BJABDR5 DEF+DR5 cells, however, the cytotoxic potential was restored (FIG. 8F). The cytotoxic effects of these TRAIL variants on noncancerous human umbilical vein endothelial cells was assessed by incubating these cells in the presence of 100 ng/ml TRAIL or TRAIL variants. However, no cytotoxic effects were observed for TRAIL and the receptor-selective TRAIL variants (data not shown).

Taken together, the results obtained with the Colo205, ML-1, A2780, and BJAB cell lines show that the biological activity of the D269H, D269HE195R, and D269H/T214R variants is specifically directed toward the DR5 receptor and that these TRAIL variants have a high efficiency in inducing apoptosis in colon and ovarian cancer cell lines.

### Example 7

### DR5-Selective TRAIL Variants Are More Effective In Tumor Cell Killing

Colo205 cells express all four TRAIL receptors, and are sensitive to TRAIL-induced apoptosis, which is mediated predominantly via DRS in these cells¹⁰. In order to compare the response of Colo205 cells to wtTRAIL and DR5-selective TRAIL variants (D269H, D269HE195R), Colo205 cells were cultured in RPMI 1640 medium supplemented with 10% fetal calf serum, 2 mM L-glutamine, 50 U/ml penicillin and 5 mg/ml streptomycin and 10 mM sodium pyruvate and seeded at 2×10⁵ cells/ml concentration 24 h before treatment (all reagents were from Sigma-Aldrich unless otherwise stated). Cells were cultured in a humidified atmosphere at 37° C. and 5% CO₂ and treated with 2-30 ng/ml of wtTRAIL, D269H or D269HE195R for 3 h to determine growth response curves (FIG. 10A). Induction of cell death was measured by Annexin V assay, wherein externalization of phosphatidyl serine (PS) on the plasma membrane of apoptotic cells was detected using Annexin V-FITC (IQ Corporation, Groningen, The Netherlands). Briefly, cells were trypsinized and were then collected by centrifugation at 400 g, washed in ice-cold calcium buffer (10 mM HEPES/NaOH, pH 7.4, 140 mM NaCl, 2.5 mM CaCl₂) and incubated with Annexin V-FITC for 15 min in the dark prior to acquisition on a FACSCalibur flow cytometer (Becton Dickinson).

As shown in FIG. 10A, the two DR5-selective TRAIL-variants displayed similar cytotoxic potential, which was 2.7-4.2 fold higher than that of wtTRAIL based on the IC50 values (determined from the linear range of the dose response curve). Caspase activation in response to WT TRAIL and DRS-selective variants was also measured and showed that 2.9-4.1 fold lower concentrations of the DRS-selective variants were sufficient to induce the same level of caspase activity (FIG. 10B). The higher DEVDase activity induced by the DR5-selective mutants correlated with stronger pro-caspase-8 processing studied after 3 h treatment with the same dose range of wtTRAIL and DR5 variants by Western blotting, wherein after treatment, cells were lysed in 100 μl whole cell lysis buffer (20 mM HEPES, pH 7.5, 350 mM NaCl, 1 mM MgCl₂, 0.5 mM ethylenediamine-tetraacetic acid (EDTA), 0.1 mM ethylene glycol bis(2-aminoethyl ether)-N,N,N′N′-tetraacetic acid (EGTA), 1% Igepal-630, 0.5 mM dithiothreitol (DTT), 100 μm phenylmethylsulphonyl fluoride (PMSF), 2 μg/ml pepstatin A, 25 μM N-Acetyl-Leu-Leu-Nle-CHO (ALLN), 2.5 μg/ml aprotinin and 10 μM leupeptin for 15 min on ice. Protein samples were denatured in Laemmli sample buffer and boiled for 5 min. Proteins were separated by 10% SDS-PAGE and transferred onto nitrocellulose membrane. Membranes were blocked for 1 h in PBS containing 0.05% Tween20 and 5% (w/v) non-fat dried milk. The membranes were then incubated for 1 h at room temperature with antibodies to actin (1:500; Sigma) or overnight at 4° C. with antibodies to caspases-3 and -8 (1:1,000; Cell Signaling Technologies). This was followed by 2 h incubation at room temperature with appropriate secondary antibodies (1:5,000 for all antibodies). Protein bands were visualized using Supersignal Ultra Chemiluminescent Substrate (Pierce) on X-ray film (Agfa). The result of the Western blot is shown in FIG. 10C.

A2780 ovarian carcinoma cells were cultured in DMEM with the same supplements as for Colo205 cells and seeded at 3.5×10⁵ cells/ml concentration 24 h before treatment.

In order to determine the cell surface expression of each of the TRAIL receptors on A2780 carcinoma cells, cells were washed twice in PBS containing 1% BSA and then incubated with monoclonal antibodies to DR4, DR5, DcR1 or DcR2 (Alexis) for 40 min. After two wash steps with PBS/BSA, anti-mouse IgG-FITC (Sigma) secondary antibody was added for 30 min. All incubations were carried out on ice. Negative controls contained isotype control antibody. Cells were analyzed by a FacsCalibur flow cytometer. A2780 carcinoma cells were found to express high levels of DR5, DcR1 DcR2 and no detectable DR4 on their surface and to be partially resistant to wtTRAIL-induced apoptosis (FIGS. 11 & 13A). Similar to Colo205 cells, D269HE195R appeared to be a more effective inducer of apoptosis than wtTRAIL in A2780 cells. While wtTRAIL could not induce more than 24.3±4.5% cell death even at the highest concentration tested (250 ng/ml), 10 ng/ml D269HE195R was sufficient to induce 30.6±2.5% apoptosis measured by Annexin V assay as described above (FIG. 13A). Study of effector caspase activation by DEVDase assay, wherein Cell lysates (25 μl) were transferred to a microtiter plate and snap-frozen over liquid nitrogen. To initiate the reaction, 50 μM of the caspase substrate carbobenzoxy-Asp-Glu-Val-Asp-AMC (7-amino-4-methylcoumarin) (DEVD-AMC) (Peptide Institute Inc., Osaka Japan) in assay buffer (100 mM Hepes buffer, 10% sucrose, 0.1% 3[(3cholamidopropyl)-dimethylammonio]-1 propanesulphonate (CHAPS), 5 mM DTT and 0.0001% Igepal 630, pH 7.25) was added to cell lysates. Substrate cleavage leading to the release of free AMC was monitored at 37° C. at 60 sec intervals for 25 cycles using a Wallac multilabel counter (excitation 355 nm, emission 460 nm). Enzyme activity was expressed as nmol AMC released/min/mg protein, and pro-caspase-8 processing by Western blotting confirmed this result (FIGS. 13B & 13C). In summary, selective activation of the DR5 receptor resulted in stronger pro-caspase-8 processing that correlated with more cell death in tumor cell lines with functional DR5 receptor, indicating that the enhanced pro-apoptotic potential may be due to more efficient receptor activation. This could be due to a combination of higher affinity and specificity for DR5 and reduced binding to the decoy receptors. To test this hypothesis we tested the interaction of our variants with DcR1 and DcR2.

### Example 8

### DR5-Selective Variants Bind Less Effectively To DcR1 Than DcR2

A surface plasmon resonance (SPR) based receptor binding assay was carried out to compare the binding affinity of wtTRAIL to D269H and D269HE195R towards immobilized DcR1- and DcR2-Ig fusion proteins (FIG. 12). Binding experiments were performed at 37° C. using a surface plasmon resonance-based biosensor (Biacore 3000, Biacore AB, Uppsala, Sweden) as previously described¹⁰. In short, DcR1-Ig and DcR2-Ig receptor chimeras (R&D Systems) were captured at a 35 μl/min flow rate using a protein A (Sigma) modified CM5 sensor chip (Biacore). Receptor chimeras were captured at a level of ˜500-800 response units (RU). Purified wtTRAIL and TRAIL variants were injected in three-fold at concentrations ranging from 250 nM to 2 nM at 70 μl/min flow rate using PBS (pH 7.4) (Invitrogen) supplemented with 0.005% vol/vol P20 (Biacore) as running and sample buffer. Binding of ligands to the receptors was monitored in real-time. Between injections the protein A sensor surface was regenerated using a 30s pulse of 0.1 M glycine 0.5 M NaCl pH 3. Due to the very slow dissociation of the TRAIL-receptor complex, only pre-steady state binding data could be obtained. In order to obtain data that represent proper high-affinity complex formation, the response at each concentration was recorded 30 sec after the end of the injection. The response data (in response units) as a function of TRAIL concentration were fitted using a four parameter equation to give an apparent pre-steady state affinity constant.

Affinity of the ligands was assessed by measuring receptor binding in a concentration range of 0.1-250 nM. The binding of D269H and D269HE195R towards DcR1 was approximately 20 fold reduced when compared to wtTRAIL (FIG. 12A). Binding of D269H and D269HE195R toward DcR2 was also reduced, however the effect was much less pronounced compared to the reduction in binding observed with the DcR1 (FIG. 12B).

To further assess the antagonistic effect of the decoy receptors, wtTRAIL, D269H and D269HE195R were incubated for 30 min with increasing concentrations of soluble, recombinant DcR1-Ig (sDcR1) and DcR2-Ig (sDcR2), prior to addition to Colo205 cell cultures at 10 ng/ml final ligand concentration. Induction of cell death was assessed by Annexin V assay 3 h after treatment (FIG. 14), as described above. Pre-incubation of wtTRAIL with 0.5 μg/ml sDcR1 completely blocked its pro-apoptotic activity. Pre-incubation of D269H or D269HE195R with sDcRl on the other hand failed to reduce their pro-apoptotic activity even at the highest concentration used (2 μg/ml, FIG. 14A). Pre-incubation with sDcR2, similar to sDcR1, was able to block apoptosis induction by wtTRAIL. sDcR2 was also able to block apoptosis induced by D269H, although only at a two fold higher concentration than for wtTRAIL. sDcR2 was even less effective against D269HE195R; no complete protection could be achieved even with the highest concentration applied (FIG. 14B). In contrast, pre-incubation of the DR5-selective variants with soluble DR5 resulted in complete blockage of their pro-apoptotic activity. (FIG. 14C). This confirms that the observed differences in blocking efficacy are due to reduced ability of the decoy receptors to bind and inhibit D269H and D269HE195R.

The above results were confirmed by pre-incubating Colo205 cells for 1 h with increasing concentrations of neutralizing antibodies to DcR1 and DcR2, prior to treatment with a dose of wtTRAIL (20 ng/ml) or D269HE195R (4 ng/ml) that induced approximately 50% cell death. Apoptotic cell death was measured 3 h post-treatment using staining Annexin V assay (FIG. 15), as described above. Increase in apoptosis was expressed as fold increase compared to the level of apoptosis caused by the ligands alone, in the absence of the neutralizing antibodies. When cells were pre-treated with DcR1 neutralizing antibody, a 1.51±0.15 fold increase in apoptosis was detected in wtTRAIL-treated cells with no increase in D269HE195R treatment. Neutralization of DcR2 led to a 1.22±4.07 fold increase in wtTRAIL-induced apoptosis, but no increase of apoptosis after D269HE195R treatment. Combination of neutralizing antibodies to DcR1 and DcR2 resulted in a further and nearly additive 1.68±0.12 fold increase in wtTRAIL-induced cell death, but not by D269HE195R (FIG. 14D). These results point out that the potency of wtTRAIL to induce apoptosis is significantly limited by the decoy receptors expressed on the cell surface. In contrast, DR5-selective TRAIL variants are able to avoid sequestration by the decoy receptors and thus display a higher pro-apoptotic potential.

Previous studies found that decoy receptor expression, either total cellular expression, or on the cell surface, did not correlate with TRAIL-sensitivity. This can be due to the presence of intracellular inhibitors of apoptosis (c-FLIP, Bcl-2, phosphorylated Akt, etc.) or compartmentalization of the receptors²⁰. Results of the current study found that neutralization of DcR1 and DcR2 could increase TRAIL-induced apoptosis (by 1.5 fold and 1.2 fold by DcR1- and DcR2-neutralization in Colo205, respectively). In other words, in the presence of the decoy receptors, the apoptosis-inducing potential of wtTRAIL is reduced by approximately 40% (100%/(1.5+1.2)) in Colo205 cells. This is the first data showing that endogenously expressed decoy receptors limit the apoptosis-inducing ability of wtTRAIL. Neutralization of the decoy receptors however failed to reduce D269HE195R-induced apoptosis, again demonstrating that the DR5-variant can escape neutralization mediated by the decoy receptors. Although high concentration of soluble DcR2 was able to limit the activity of D269HE195R, cell surface expressed DcR2 was not sufficient to reduce apoptosis-induction by D269HE195R. This may be due to a different ratio or of DRS:DcR2 or difference in structure of full length, surface expressed DRS and DcR2 compared to the soluble, recombinant, Fc-fusion receptors.

Although our results cannot exclude the ligand-independent model proposed by Clancy and colleagues²¹, they are more in support of the ligand dependent model suggested by Merino and co-workers²². Due to their selectivity towards one receptor, one can speculate that the DR5-selective mutants favour the formation of homomeric DR5 complexes, rather than non-functional, heteromeric complexes between DR5 and decoy receptors, which enhances the pro-apoptotic activity of the ligand.

### Example 9

### DR5-Selective Variants Are Not Toxic Towards Non Transformed Cells

The importance of decoy receptors in protecting non-transformed cells against TRAIL has been suggested by many reports.³⁷ Thus, the toxic effect of the DR5-selective variants on non-cancerous cells was assessed. Human dermal fibroblasts were grown in low glucose DMEM (Sigma) supplemented with 10% fetal calf serum, 50 U/ml penicillin and 5 mg/ml streptomycin; human umbilical vein endothelial cells (HUVEC, PromoCell) were cultured in Endothelial Cell Growth Medium with SupplementMix (PromoCell). Fibroblasts and HUVEC cells were seeded at 1.3×10⁵ cells/ml 24 h before treatment with increasing concentrations of TRAIL and DR5-selective variants, D269H and D269HE195R (50-500 ng/ml) for 24 h and cell viability was measured using MTT assay, wherein 200 μg/ml MTT (3-(4,5-dimethylthiazo1-2-yl)-2,5-diphenyl-tetrazolium bromide) was added to control and treated cells and incubated for 3 h at 37° C. The reaction was stopped and the purple formazan precipitate formed was dissolved using dimethyl sulfoxide (DMSO) and the colour intensity was measured at 550 nm on a Wallac multilabel counter. The control value corresponding to untreated cells was taken as 100% and the viability of treated samples was expressed as a percentage of the control. (FIG. 15). Neither wtTRAIL nor the DR5-selective variants had any effect on cell viability in primary cultures of human fibroblasts (FIG. 15A) or the endothelial cell line HUVEC, indicating only a limited role for the decoy receptors in protecting non-cancer cells against TRAIL induced apoptosis (FIG. 15B).

### Example 10

### DR5-Selective Variants Activate DR5 17-Fold Faster Than wtTRAIL

Although decoy receptor neutralization increased the apoptosis-inducing potency of wtTRAIL by 1.68 fold, this increase alone may not fully explain the 2.7-4.2 fold higher pro-apoptotic activity of D269HE195R. D269HE195R triggered stronger pro-caspase-8 processing at lower concentration than wtTRAIL, suggesting an enhanced ability to activate DR5. To compare the ability of wtTRAIL and D269HE195R to activate the death-inducing TRAIL receptors, Colo205 cells were incubated with 30 ng/ml concentration of the ligands (at which concentration both wtTRAIL and D269HE195R induce maximal cell death) for varying times (5-180 min), after which the ligands were washed out and the incubation was continued in normal growth medium to 180 min in total, when the cells were harvested and as a marker of receptor activation, pro-caspase-8 processing was measured. Treatment with D269HE195R resulted in significant pro-caspase-8 processing already after 5 min of incubation, while 60 min was required for a comparable level of pro-caspase-8 processing by wtTRAIL (FIG. 16A).

The faster kinetics of receptor activation was reflected in apoptosis induction. Colo205 cells were treated with 30 ng/ml wtTRAIL or D269HE195R for the times indicated, after which the ligand was washed out and the incubation continued up to 240 min in total in normal growth medium. The cells were harvested and cell death was quantified with Annexin V labelling (FIG. 16B), as described above. The data was fitted with an interpolating spline using the software MatlabR. From the fitted curve the incubation time required for 50% efficacy was determined to be 60.81 min for wtTRAIL. The same value for D269HE195R was 3.55 min, showing that receptor activation by D269HE195R occurs 17 fold faster than with wtTRAIL. This could be the result of faster binding kinetics for the designed variants and/or more efficient binding if we assumed that heterogenic receptors are present in the cell or could be formed upon ligation of TRAIL to a receptor (ie a complex of DR4-DR5-DcRI). In the latter case, the designed mutants will preferentially make complexes only with DR5 receptors and therefore induced efficient activation.

To determine whether this faster receptor activation was due to faster receptor binding by D269HE195R compared to wtTRAIL, rather than difference in the capability of receptor activation between the two ligands, Colo205 cells were pre-incubated with wtTRAIL for 5 or 10 min, washed and then D269HE195R was added to the cells and incubated for another 5 or 10 min (enough to induce cell death) and removed. The cells were then incubated to a total of 180 min in normal growth medium and then the level of cell death was determined (FIG. 16C). The results show that pre-incubation with the wtTRAIL did not affect apoptosis induction by the double mutant. If the slow kinetics of apoptosis induction by the wtTRAIL was due to the formation of non-active heterogenic complexes with the 4 receptors, but not to slower binding to the receptors, we would have expected complete inhibition of apoptosis by the mutant. If on the other hand the binding kinetics of the wtTRAIL is very slow, then short incubation periods will result in almost no receptors bound and, subsequently, no inhibition of the D2691-1E195R's higher pro-apoptotic potency. Clearly our results support the latter hypothesis.

The results gained suggest that D269HE195R binds to DR5 with a faster kinetics as wtTRAIL as pre-incubation with wtTRAIL either for 5 or 10 min failed to reduce D269HE195R-induced apoptosis. These results also indicate that the difference between the ability of the two ligands to induce apoptosis is not due to a difference in their ability to activate the receptor after binding to it.

### Example 11

### TRAIL And DR5-Selective Variants Exhibit Higher Synergism With Aspirin In Colo205 Cells And Tumors Induced By Inoculation With Colo205 Cells

Various chemotherapeutics have been reported to increase the sensitivity of tumour cells towards TRAIL and it often correlated with induction of DR5. Many of these treatments however also induce the expression of the decoy receptors, which may limit the degree of synergism/amount of cell death induced. The efficacy of the DR5-selective variant D269HE195R in one such model was tested. The combined effect of aspirin with wtTRAIL or D269HE195R in Colo205 cells was studied (FIG. 22). The effect of aspirin on TRAIL receptor expression and induction of caspase activation was monitored. Treatment with 5 mM aspirin led to increased expression of DR5, as well as DcRI and DcR2 on the cell surface (FIG. 22A). 24 h treatment with aspirin increased the sensitivity of the cells to wtTRAIL, however, the sensitisation was much more pronounced with D269HE195R (FIG. 22B). These results highlight that selective receptor activation with high affinity TRAIL variants leads to pronounced increase in tumour cell killing.

Further in vivo experiments were conducted to investigate the synergistic effect of aspirin and TRAIL, and the difference between the synergistic effects of wtTRAIL and D269HE195R.

In a first experiment 2 concentrations of Colo205 cells were used for inoculation. Tumor growth was followed for a period of 6 weeks. The concentrations used were 2 and 5×10⁶ cells in 200 μl of medium. The tumor growth is shown in FIG. 20. The mice that were inoculated with 2×10⁶ cells did not get a lot of tumor growth, the tumors reached an average volume of ˜80 mm³. The tumors starting from 5×10⁶ cells developed a nice tumor, average volume ˜750 mm³. For further experiments this last concentration was used for inoculation.

A second study was conducted using aspirin alone to determine the concentration which did not give side effects and which could be combined with TRAIL. In this study 2 concentrations of aspirin, 40 and 200 mg/kg, were used. Both were given for a 2×5 days period with 2 days in between. FIG. 21 shows the data form this experiment. As can be seen from the graph there was no difference in effect between the 2 concentrations or the control group. Also no side effects were found at either concentration. For the combination study we used 200 mg/kg aspirin.

In a third study the combination between TRAIL and aspirin was tested. Both wtTRAIL and DR5-specific D269HE195R were used. Both were tested in doses of 1 and 5 mg/kg, given by intraperitoneal injections for 2×5 days (day 1-5 and 8-12) with daily doses of aspirin (in the same syringe as protein). Data is only presented until day 13, because of too many fluctuations and mice dying after this day.

As can be seen from FIG. 24, neither of the two concentrations of wtTRAIL give an anti-humor effect, whereas treatment with mutant TRAIL reduces the tumor growth. FIG. 24 shows the effect of wtTRAIL both doses in single and combination therapy.

For both concentrations of wtTRAIL there seems to be an additive effect of the combination of wtTRAIL with aspirin. FIG. 25 shows the effect of combination of D269HE195R with aspirin. For the lowest concentration of TRAIL there seems to be an additive effect of the combination with aspirin, for the 5 mg/kg TRAIL dose this effect is not visible. Possibly because the effect caused by 5 mg/kg D269HE195R is the maximum effect that can be achieved at this dose.

Statistical tests were performed using an independent samples T-test and even when these experiments were performed with a very small number of mice per group (<5), significance was found between WT 5 mg/kg vs WT 5 mg/kg+aspirin and between WT 5 mg/kg and D269HE195R 5 mg/kg.

### Example 12

### Cell Killing of Three Colon Carcinoma Cell Lines By wtTRAIL And DR5-Selective TRAIL

Three colon carcinoma cell lines were used, Colo205, LoVo and SW948. All three cell lines express the four membrane-bound TRAIL receptors. Sensitivity of these cell lines to wtTRAIL and the DR5-specific variant D269HE195R was compared initially as a single agent. The sensitivity pattern is illustrated in Table 4, with + indicating minimally sensitive, ++ moderately sensitive and +++ very sensitive.

Colo205 and LoVo showed higher sensitivity to the DR5-selective variant compared to wtTRAIL. SW948 cells showed higher sensitivity to wtTRAIL compared. Further studies have shown that in SW948 cells selectively transmits the death inducing signal predominantly through the DR4 receptor (data not shown).

Currently used chemotherapeutics, namely 5-Flurouracil and Aspirin have been tested in combination with wtTRAIL and DR5-selective variant. In Colo205 and LoVo cells both 5-FU and Aspirin enhanced apoptosis induced by wtTRAIL and DR5-selective variants (FIGS. 24, 27, 28 & 33-36).

### Example 13

### TRAIL And DR5-Selective Variants Exhibit Higher Synergism With Aspirin In Colo205 Cells And Tumors Induced By Inoculation With Colo205 Cells

Various chemotherapeutics have been reported to increase the sensitivity of tumor cells towards TRAIL and it often correlated with induction of DR5. Many of these treatments however also induce the expression of the decoy receptors, which may limit the degree of synergism/amount of cell death induced. The efficacy of the DR5-selective variant D269HE195R in one such model was tested. The combined effect of aspirin with wtTRAIL or D269HE195R in Colo205 cells was studied (FIG. 18). The effect of aspirin on TRAIL receptor expression and induction of caspase activation was monitored. Treatment with 5 mM aspirin led to increased expression of DR5, as well as DcR1 and DcR2 on the cell surface (FIG. 18A). 24 h treatment with aspirin increased the sensitivity of the cells to wtTRAIL, however, the sensitisation was much more pronounced with D269HE195R (FIG. 18B). These results highlight that selective receptor activation with high affinity TRAIL variants leads to pronounced increase in tumor cell killing.

Further in vivo experiments were conducted to investigate the synergistic effect of aspirin and TRAIL, and the difference between the synergistic effects of wtTRAIL and D269HE195R TRAIL.

In a first experiment 2 concentrations of Colo205 cells were used for inoculation. Tumor growth was followed for a period of 6 weeks. The concentrations used were 2 and 5×10⁶ cells in 200 μl of medium. The tumor growth is shown in FIG. 19. The mice that were inoculated with 2×10⁶ cells did not get a lot of tumor growth, the tumors reached an average volume of ˜80 mm3. The tumors starting from 5×10⁶ cells developed a nice tumor, average volume ˜750 mm3. For further experiments this last concentration was used for inoculation.

A second study was conducted using aspirin alone to determine the concentration which did not give side effects and which could be combined with TRAIL. In this study 2 concentrations of aspirin, 40 and 200 mg/kg, were used. Both were given for a 2×5 days period with 2 days in between. FIG. 20 shows the data form this experiment. As can be seen from the graph there was no difference in effect between the 2 concentrations or the control group. Also no side effects were found at either concentration. For the combination study we used 200 mg/kg aspirin to be sure we would see an additive effect if there is any.

In a third study the combination between TRAIL and aspirin was tested. Both wtTRAIL and DR5-specific D269HE195R TRAIL were used. Both were tested in doses of 1 and 5 mg/kg, given by intraperitoneal injections for 2×5 days (day 1-5 and 8-12) with daily doses of aspirin (in the same syringe as protein). Data is only presented until day 13, because of too many fluctuations and mice dying after this day.

As can be seen from FIG. 21, both concentrations of wtTRAIL do not give an anti-tumor effect. Both concentrations of mutant TRAIL seem to have some positive effect on the tumor growth. FIG. 23 shows the effect of wtTRAIL both doses in single and combination therapy.

For both concentrations of wtTRAIL there seems to be an additive effect of the combination of wtTRAIL with aspirin. FIG. 24 shows the effect of combination of D269HE195R TRAIL with aspirin. For the lowest concentration of TRAIL there seems to be an additive effect of the combination with aspirin, for the 5 mg/kg TRAIL dose this effect is not visible. Possibly because the effect caused by 5 mg/kg D269HE195R TRAIL is the maximum effect that can be achieved at this dose.

Statistical tests were performed using independent samples T-test and significance was found only between WT 5 mg/kg vs WT 5 mg/kg +aspirin and between WT 5 mg/kg and D269HE195R 5 mg/kg.

### Example 14

### Cell Killing of Three Colon Carcinoma Cell Lines By wtTRAIL And DR5-Selective TRAIL

Three colon carcinoma cell lines were used, Colo205, LoVo and SW948. All three cell lines express the four membrane-bound TRAIL receptors. Sensitivity of these cell lines to wtTRAIL and the DR5-specific variant D269HE195R was compared initially as a single agent. The sensitivity pattern is illustrated in Table 4, with + indicating minimally sensitive, ++ moderately sensitive and +++ very sensitive.

Colo205 and LoVo showed higher sensitivity to the DR5-selective variant compared to wtTRAIL. SW948 cells showed higher sensitivity to wtTRAIL compared. Further studies have shown that in SW948 cells selectively transmits the death inducing signal predominantly through the DR4 receptor (data not shown).

Currently used chemotherapeutics, namely 5-Flurouracil and Aspirin have been tested in combination with wtTRAIL and DR5-selective variant. In Colo205 and LoVo cells both 5-FU and Aspirin enhanced apoptosis induced by wtTRAIL and DR5-selective variants (FIGS. 17, 22, 25, 26 & 31-34). In SW948 cells, 5-FU enhanced sensitivity to wtTRAIL but not to the DR5-selective variant (FIGS. 27-30). Interestingly, Aspirin enhanced the effect the DR5-selective variant in SW948 cells, suggesting that chemotherapeutics can modulate DR5 sensitivity in cells.

To determine the antitumor efficacy of TRAIL-DR5 versus TRAIL alone and in combination with aspirin in colon cancer Colo205 xenografts, we first established tumor growth characteristics in mice and in a second pilot tested TRAIL and TRAIL-DR5 in mice bearing Colo205 xenografts. Experiments demonstrated a nice response of Colo205 xenografts to TRAIL-DR5 and to a lesser extent to TRAIL. Pathological analyses of the mice and the tumors suggested that the commercially obtained mice were not in good condition (data not shown).

### Example 15

### Cell Killing of Three Cervical Carcinoma Cell Lines By wtTRAIL And DR5-Selective TRAIL

Three cervical carcinoma cell lines were used: HeLa, Caski and SiHa. All three cell lines express all four surface-expressed TRAIL receptors. Sensitivity of these cell lines to wtTRAIL and the, DR5-specific/selective variant D269HE195R was compared initially as a single agent. The sensitivity pattern is illustrated in the Table 5, with− indicating resistant, +: minimally sensitive, ++: moderately sensitive, +++: very sensitive.

Both HeLa and Caski cells showed similar sensitivities to the DR5-selective variant and wtTRAIL (FIGS. 40-43 & 48-51). SiHa cells on the other hand were resistant to both wtTRAIL and DRS-selective variant (FIGS. 44-47).

Common therapies of cervical cancer are radiotherapy and proteasome inhibition (Bortezomib). Both radiotherapy and Bortezomib were tested in combination with wtTRAIL and DR5-selective variant. Both agents enhanced apoptosis in HeLa cells induced by both wtTRAIL and DR5-selective variant. Radiotherapy had no enhancing effect in SiHa and Caski cells, but combination of bortezomib with wtTRAIL or DR5-slective variant resulted in increased tumor cell death. In all cases, where sensitisation was detected, the degree of sensitisation was higher with the DRS-selective variant than wtTRAIL.

To determine the antitumor efficacy of TRAIL-DRS versus TRAIL alone and in combination with radiotherapy, bioluminescent HeLa xenografts have been established. A luciferase positive HeLa cell line was produced to allows monitoring of the onset of tumor growth in mice before they are visible under the skin (data not shown).

### Example 16

### TRAIL And DR5-Selective Variants Are Not Toxic To Non-Transformed Cells

The importance of decoy receptors in protecting non-transformed cells against TRAIL has been proposed by many reports. Thus, the toxic effect of the DR5-selective variants on non-cancerous cells was assessed. Human dermal fibroblasts and human umbilical vein endothelial cells (HUVEC) were treated with increasing concentrations of wtTRAIL and DRS-selective variants, D269H and D269HE195R (50-500 ng/ml) for 24 h and cell viability was measured using MTT assay (data not shown), as described above. Neither wtTRAIL nor the DR5-selective variants had any effect on cell viability in primary cultures of human fibroblasts (FIG. 37A) or the endothelial cell line HUVEC (FIG. 37B).

### Example 17

### Further Characterisation of DR5-Specific TRAIL Variants

The designed TRAIL variants were made by site-directed mutagenesis of the extra-cellular domain of human TRAIL, comprising amino acids 114-281. TRAIL proteins were all produced from pET15b as recombinant, soluble proteins in Escherichia coli and purified in three chromatographic steps¹⁰. The purified proteins were characterised for binding to the various receptors in ELISA assays and in a pre-steady state approach using Surface Plasmon Resonance (SPR) assays, and furthermore by their ability to induce cell death in cell lines with different TRAIL receptor expression patterns.

Several DR5-specific variants have been designed and characterised, amongst which is variant D269HE195R¹⁰. The two amino acid changes in this variant occur at the interface of TRAIL and receptor, as depicted in FIG. 35. The positions of residues E195 and D269 are indicated in a top and side view of the trimer-trimer complex of TRAIL and DR5.

FIG. 35A shows a typical sensorgram when TRAIL binds to immobilised TRAIL receptor. Especially at low concentrations, the curves do not show significant dissociation. In other experiments dissociation was continued for periods of up to 2,000 sec, with similar results. As a consequence it is essentially impossible to obtain a dissociation rate constant and thus to calculate the affinity from the association and dissociation rate constants. We therefore chose to use the pre-steady state approach to analyse the data¹⁰. SPR analysis of the ability of this mutant in comparison to that of wtTRAIL to bind to immobilised receptors is depicted in FIG. 35B. The binding studies demonstrate the remarkable success of the design: the affinity of D269HE195R for DR5-Ig has been increased several fold and the affinity for DR4-Ig is almost abolished. With these two changes this variant has become an TRAIL variant that binds to DR5 with high selectivity and affinity.

The DR5-selectivity of D269HE195R was also demonstrated in cancer cell lines. In FIG. 40, the apoptotic activity of D269HE195R is shown in comparison to wtTRAIL in colon carcinoma cancer cell line Colo205, where living cells were stained with a soluble tetrazolium-based MTS proliferation reagent (Promega). Colo205 cells are sensitive to TRAIL induced apoptosis mainly via the DR5 receptor. Indeed, our variant D269HE195R is efficient at a several fold lower concentration than needed for wtTRAIL. The increase in efficiency is not only demonstrated by a dose-response curve, but can also be observed in time. In FIG. 41, induction of cell death in Colo205 cells upon treatment with 25 ng/ml of wtTRAIL or D269HE195R is demonstrated in time. Clearly, induction of cell death proceeds faster with the DR5-specific variant. A better performance of D269HE195R could also be demonstrated in apoptosis-specific assays e.g. Annexin V staining and caspase assays³⁹. Several other cell lines have been tested, resulting in an improved efficiency in DR5-sensitive cell lines, e.g. human ovarian carcinoma cell line A2780 and the Burkitt-like lymphoma cell line BJAB, whereas hardly any apoptosis could be observed in cell lines that are essentially DR4-responsive e.g. chronic myeloid leukaemia cell lines ML1 and EM-2¹⁰.

Studies with xenografted athymic nude mice carrying tumors of human ovarian A2780 cells were performed by the group of Steven de Jong and Liesbeth de Vries at the University Medical Centre Groningen³⁴.

### Example 18

### Mathematical Modelling of Ligand-Receptor Interaction

To investigate the mechanism of faster receptor binding kinetics of D269HE195R and to be able to dissect the contribution of each death-inducing and decoy receptor to the induction of apoptosis, we simulated the ligand-receptor interaction using a mathematical model. The model simulates the interactions occurring on the surface of Colo205 cells upon exposure of the cells to wtTRAIL or DR5-variant. The model describes the binding of the ligand (wtTRAIL or D269H/E195R) to the receptors in a stepwise fashion and allows for the formation of both homomeric (e.g. TRAIL-3DR5) and heteromeric receptor complexes (e.g. TRAIL-2DR5-DcR2).

Receptor binding of TRAIL and D269HE195R on the cell surface was simulated using a mathematical model describing all possible binding events. Both the formation of homomeric (e.g. TRAIL-3DR5) and heteromeric ligand-receptor complexes (e.g. TRAIL-2DR5-DcR2) were allowed and binding was simulated in a stepwise fashion. On rates and off rates measured for TRAIL binding to monomeric DR4, DR5, DcR1 and DcR2 receptors were taken from the report of Lee and colleagues⁴⁰. In contrast to the more commonly used dimeric TRAIL-receptor-IgG Fc chimeric constructs, Lee et al., employed monomeric TRAIL-receptor constructs. This allowed us to derive stepwise binding constants and to model heteromeric receptor-ligand interactions as described in details in Results. Stepwise constants for going from a single ligand-bound receptor, via two ligand-bound receptors, to the complex consisting of 3 receptors bound by a trimeric ligand were estimated in the following way: the kₒₙ reported by Lee et al., was assigned to the first binding event. The first association step is entropically the most unfavourable one due to loss of rotational degrees of freedom when going from an unbound state to a bound state. To compensate for this entropic penalty in the first step, the kₒₙ for the second and third step was increased by 2 kcal/mol. The reported k_{off} was assigned to the third step. To compensate for avidity effects present in the second and third step (TRAIL bound to two or three receptors) the k_{off} for the second and the first step was increased by a factor ten and hundred, respectively. The effect of avidity on the apparent off-rate will be larger upon binding two receptor subunits instead of one than for binding three subunits instead of two, hence the difference in factors used. In the case of homomeric TRAIL-3DR5 and TRAIL-3DR4 complexes, an extra step to an “activated” receptor complex was introduced in order to mathematically describe the assembly of the intracellular DISC. Formation of the DISC reduces the likelihood of complex disassembly and participation in rebinding events with other free death or decoy receptors. As mentioned before, DcR1 does not contain an intracellular domain and DcR2 only contains a truncated Death domain, no “activation” step was added for the decoy receptors (Table 1a).

The number of DR5 receptors on the surface of Colo205 was estimated by comparing the receptor expression levels on the surface of Colo205 to MD MBA-231 cells and subsequently relating this ratio to the number of surface expressed receptors determined for MD MBA-231 cells by Kim and co-workers⁴⁰. Apparent affinities for D269HE195R were taken from van der Sloot et al.,¹⁰. The increase in affinity of D269HE195R for DR5 was completely attributed to the kₒₙ, as changes in off-rate in the SPR sensorgram between wtTRAIL and D269HE195R could not be calculated due to minimal and very slow rate of ligand dissociation. We choose to use a factor 5 difference in kₒₙ between wtTRAIL and D269HE195R in order not to underestimate the effect of an increase in kₒₙ for DR5. The decrease in affinity for the other receptors was equally attributed to the kₒₙ and the k_{off} because SPR sensorgrams of D269HE195R revealed both changes in the association phase and dissociation phase relative to the sensorgrams of wtTRAIL. The “activation” step for the homomeric death receptor complexes was considered independent from the ligand used, this the same “kₒₙ and k_{off}” values were used as for wtTRAIL (table 1b). The model was simulated using the stochastic next subvolume method (Elf and Ehrenberg) as implemented in SmartCell version 4.2 (2, 8) (http://smartcell.crg.es/). Receptor binding was simulated with the ligands continuously present, or by ligand removal during the course of the simulation in order to simulate the washout experiment. In the later case the system was simulated for the indicated time and then the free ligand concentration was set to zero.

When simulating the binding of wtTRAIL, the “ED₅₀” of the wtTRAIL-3DR5 complex was reached within ˜500 sec, while the ED₅₀ for DR4 was reached after ˜1,200 sec (FIG. 55A). The number of wtTRAIL-3DR5 complexes formed after 1 h was highest, while the number of TRAIL-3DR4, TRAIL-3DcR1 and TRAIL-3DcR2 were 70%, 6% and 0% of the number of TRAIL-3DR5 complexes, respectively. In contrast, the “ED₅₀” for D269H/E195R-3DR5 complex formation was reached within 20 sec and no D269HE195R-3DR4, -3DcR1 or -3DcR2 complexes were formed during 1 h of simulation (FIG. 55B). The number of D269HE195R-3DR5 complexes formed after 1 h was 125% of that of with wtTRAIL. Removing the DcR1 and DcR2 receptors from the model increased the total number of wtTRAIL-3DR5 complexes by 10% and reduced the time to reach the ED₅₀ to ˜380 sec (FIG. 55C). For wtTRAIL-3DR4 complex the “ED₅₀” was reached within 240 s in the absence of the decoy receptors and the total number of complexes increased by 14%. Absence of the decoy receptors only marginally affected the D269HE195R-3DR5 complex formation (FIG. 55D). Overall, blocking the decoy receptors was not enough for wtTRAIL to reach the same kinetics of binding as D269HE195R, which is in agreement with the experimental decoy-blocking assay. A control experiment where wtTRAIL's kinetic constant for DR5 was increased to that of D269HE195R revealed that increasing the kinetics for DR5-binding alone is not enough to reach same activity as the DR5 selective variant. Instead, the observed effect is a combination of increased affinity for DR5 and decreased affinity for DR4, DcR1 and probably DcR2 (FIG. 56A). This was indicated further when the formation of heterotrimeric complexes was studied. wtTRAIL triggered a much higher amount of heteromeric receptor complexing, especially during the first 30 min of incubation. This receptor pool appeared to be dynamically changing and gradually disappearing, due to rearrangement into homomeric complexes. D269HE195R on the other hand resulted in a much lower level of receptor hetero-trimerization and only during the first 5 min of the incubation (FIG. 56D, E).

Simulating the wash out experiment showed that D269HE195R already reached maximum binding after 5 min of incubation and that there is no significant redistribution of D269HE195R once it is bound to DR5. On the other hand, even after 30 min of incubation with wtTRAIL, the number of wtTRAIL-3DR5 and -3DR4 complexes is at 80% and 70% of the maximum level, respectively (FIG. 56B, C).

In summary, the mathematical simulations showed that the reduced affinity of D269HE195R for DR4, DcR1 and DcR2 in combination with an increased kₒₙ for DR5, all contributed to produce a 17-fold increase in the rate of homotrimeric DR5 receptor complex formation. Also, the model explains other experimental data: it shows that D269HE195R is already bound to the majority of the available DR5 receptors within 5 min; washing out of unbound D269HE195R consequently would hardly influence apoptosis induction. Moreover, the model also reveals that blocking the decoy receptors or improving the binding of wtTRAIL towards DR5 separately is not sufficient to enhance its activity to a level comparable to that of D269HE195R. If the binding of wtTRAIL to the decoy receptors is blocked, wtTRAIL can still form inactive DR4/DR5 heterotrimers, therefore only an improvement of its binding to DR5 will be sufficient to prevent the formation of inactive heteromeric complexes. This phenomenon has implications for the design of other faster working ligands; engineering faster kinetics for other promiscuous ligands cannot be achieved by only increasing the kₒₙ for the target receptor but also needs to decrease the affinity for the other receptors. Given the short in vivo half-life reported for wtTRAIL 46, faster receptor binding kinetics (and faster induction of apoptosis) can generate a considerable therapeutic advantage.

### REFERENCES CITED

1 Boppana, S. B (1996) Indian. J. Pediatr. 63 (4):447-52

2 Nishihira, J. (1998) Int. J. Mol. Med. 2 (1):17-28

3 Kim, P. K. et al (2000) Surg. Clin. North. Am. 80 (3):885-894

4 Clark, R. A. (1991) J. Cell Biochem. 46 (1):1-2

5 Flad, H. D. et al (1999) Pathobiology. 67 (5-6):291-293

6 Ashkenazi et al (1999)., J. Clin. Invest 104, 155-162

7 Ruiz de Almodovar et al., (2003), Nov 17, Manuscript M311243200

8 Wiley et al (1995)., Immunity. 3, 673-682

9 Pitti et al. (1996), J. Biol. Chem. 271, 12687-12690

10 Van der Sloot et al (2006), PNAS, 103: 8634-8639

11 Terpe K, (2003) , Appl Microbiol Biotechnol , 60:523-33

12 Gentz et al (1989). PNAS, 86:821-4

13 Wilson et al (1994). Cell, 37:767-78

14 Sambrook (1989) Molecular Cloning; A Laboratory Manual ISBN: 0879695773

15 Fernandez et al. (1998) Gene expression systems: Using nature for the art of expression ISBN: 0122538404

16 Summers et al. (1987; Texas Agricultural Experiment Station Bulletin No. 1555)

17 Zenk (1991) Phytochemistry 30:3861-3863

18 Gennaro (2000) Remington: The Science and Practice of Pharmacy 20th ed, ISBN: 0683306472

19 Hymowitz et al.(2000), Biochemistry 39, 633-640

20 Zhang et al. (2000), J. Immunol. 165, 6512-5620

21 Clancy et al. (2005) Proc Natl Acad Sci. 102, 18099-18104

22 Merino et al. (2006) Mol Cell Biol. 26, 7046-7055

23 Leslie et al. (1992) Newsletter on Protein Crystallography 26

24 Evans et al. (1993) Proceedings of CCP4 Study Weekend, 1993, on Data Collection & Processing 114-122.

25 McCoy et al. (2007) J. Appl. Cryst. 40, 658-674.

26 Jones et al. (1991) Acta Crystallogr A. 47 (pt 2), 110-119

27 Vagin et al. (2004) Acta Crystollagra D Biol Crystallogr. 60, 2184-2195

28 Hymowitz et al. (1999) Molecular Cell 4, 563-571

29 Mongkolsapaya et al. (1999) Nat. Struct. Biol. 6 (11), 1048-1053

30 Cha et al. (2000) J Biol Chem 273 (40), 31171-31177

31 Guerois et al (2002) J. Mol. Biol. 320, 369-387

32 Kiel et al. (2004) J. Mol. Biol. 340, 1039-1058

33 Schymkowitz et al. (2005) Proc. Natl. Acad. Sci. USA 102, 10147-10152

34 Duiker E. PhD thesis at the University of Groningen, The Netherlands, February 2008

35 Hougardy et al. (2006) Int J Cancer 118:1892-900.

36 Van Geelen et al. (2003) Br J Cancer, 89:363-73.

37 Marsters, et al. (1999) Recent Prog Horm Res 54, 225-34.

38 Kelley et al. (2001) J Pharmacol Exp Ther 299 (1), 31-38.

39 Szegezdi et al., manuscript in preparation

40 Kim SH et al. (2004) J Biol Chem 279, 40044-40052.

41 Lee et al (2005), Biochemical and Biophysical Research Communications, 330:1205-1212

1-11. (canceled)

12. A method of treating a mammal diagnosed with colon, ovarian or cervical cancer comprising administering a variant TRAIL protein comprising the mutation D269H and the mutation E195R to said mammal, wherein said variant TRAIL protein has superior selectivity for the death receptor 5.

13. The method according to claim 12, wherein said TRAIL variant further comprises the mutation T214R.

14. The method according to claim 12, wherein cells of said cancer express the DR5 receptor on their surface.

15. The method according to claim 13, wherein cells of said cancer express the DR5 receptor on their surface.

16. The method according to claim 12, wherein said TRAIL variant is administered in combination with a chemotherapeutic agent.

17. The method according to claim 13, wherein said TRAIL variant is administered in combination with a chemotherapeutic agent.

18. The method according to claim 16, wherein said chemotherapeutic agent increases the surface expression of the DR5 receptor on the cancer cell.

19. The method according to claim 17, wherein said chemotherapeutic agent increases the surface expression of the DR5 receptor on the cancer cell.

20. The method according to claim 16, wherein said chemotherapeutic agent is cisplatin.

21. The method according to claim 17, wherein said chemotherapeutic agent is cisplatin.

22. The method of claim 12, wherein said mammal is a human.

23. The method of claim 13, wherein said mammal is a human.

24. The method of claim 22, wherein said human is an adult.

25. The method of claim 23, wherein said human is an adult.

26. The method of claim 22, wherein said human is a child.

27. The method of claim 23, wherein said human is a child.

28. The method according to claim 12, wherein said TRAIL variant is administered intraperitoneally.

29. The method according to claim 13, wherein said TRAIL variant is administered intraperitoneally.

30. The method according to claim 12, wherein said TRAIL variant is administered in a weekly dosing regime.

31. The method according to claim 13, wherein said TRAIL variant is administered in a weekly dosing regime.


SEQUENCE LISTING (8 sequences)

SEQ ID NO: 1 - protein, 183 aa, Homo sapiens
  misc_feature: WT TRAIL
MAMMVGGSGTCVVTVSCVAVTYVYTNKMDKYSKSGACKDDSYWDNDSMNSCWVKWRVRKMRTSTSTVKNSVRRGRVAAHTGTRGRSNTSSNSKNKAGRKNSWSSRSGHSSNHRNGVHKGYYYSTYRKNTKNDKMVYYKYTSYDMKSARNSCWSKDAYGYSYGGKNDRVSVTNHDMDHASGAVG

SEQ ID NO: 2 - DNA, 1769 bp, Homo sapiens
  misc_feature: WT TRAIL
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 3 - protein, 114 aa, Homo sapiens
  misc_feature: TRAIL 114-281
MVRRGRVAAHTGTRGRSNTSSNSKNKAGRKNSWSSRSGHSSNHRNGVHKGYYYSTYRKNTKNDKMVYYKYTSYDMKSARNSCWSKDAYGYSYGGKNDRVSVTNHDMDHASGAVG

SEQ ID NO: 4 - DNA, 510 bp, Homo sapiens
  misc_feature: TRAIL 114-281
atggtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgccagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 5 - RNA, 19 nt, Artificial Sequence
  Synthetic oligonucleotide DR5 sense siRNA
gacccuugugcucguuguc

SEQ ID NO: 6 - RNA, 19 nt, Artificial Sequence
  Synthetic oligonucleotide DR5 antisense siRNA
gacaacgagcacaaggguc

SEQ ID NO: 7 - RNA, 19 nt, Artificial Sequence
  Synthetic oligonucleotide Luciferase sense siRNA
cuuacgcugaguacuucga

SEQ ID NO: 8 - RNA, 19 nt, Artificial Sequence
  Synthetic oligonucleotide Luciferase antisense siRNA
ucgaaguacucagcguaag